SEQUENCE LISTING (16 sequences)

SEQ ID NO: 1 - protein, 1457 aa, Artificial Sequence
  source: /note=“Description of Artificial Sequence Synthetic polypeptide”
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 415 aa, Homo sapiens
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 3 - protein, 1457 aa, Artificial Sequence
  source: /note=“Description of Artificial Sequence Synthetic polypeptide”
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKHHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 4 - protein, 1457 aa, Artificial Sequence
  source: /note=“Description of Artificial Sequence Synthetic polypeptide”
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKAHQAEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 5 - protein, 1453 aa, Artificial Sequence
  source: /note=“Description of Artificial Sequence Synthetic polypeptide”
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 6 - protein, 1454 aa, Artificial Sequence
  source: /note=“Description of Artificial Sequence Synthetic polypeptide”
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 7 - protein, 1474 aa, Artificial Sequence [sequence omitted]

SEQ ID NO: 8 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 9 - protein, 65 aa, Artificial Sequence
  source: /note=“Description of Artificial Sequence Synthetic polypeptide”
MIPAKDMAKVMIVMLAICFLTKSDGKSVKKRSVSEIQLMHNLGKHLNSMERVEWLRKKLQDVHNF

SEQ ID NO: 10 - protein, 115 aa, Artificial Sequence
  source: /note=“Description of Artificial Sequence Synthetic polypeptide”
MIPAKDMAKVMIVMLAICFLTKSDGKSVKKRSVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 11 - protein, 506 aa, Artificial Sequence
  source: /note=“Description of Artificial Sequence Synthetic polypeptide”
MIPARFAGVLLALALILPGTLCSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPP

SEQ ID NO: 12 - protein, 1247 aa, Artificial Sequence
  source: /note=“Description of Artificial Sequence Synthetic polypeptide”
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPP

SEQ ID NO: 13 - protein, 232 aa, Artificial Sequence
  source: /note=“Description of Artificial Sequence Synthetic polypeptide”
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 585 aa, Homo sapiens [sequence omitted]

SEQ ID NO: 15 - protein, 461 aa, Artificial Sequence
  source: /note=“Description of Artificial Sequence Synthetic polypeptide”
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLLSTKFTIYNNMFCAGFHEGGRDSCQGDSGGPHVTEVEGTSFLTGIISWGEECAMKGKYGIYTKVSRYVNWIKEKTKLT

SEQ ID NO: 16 - protein, 6 aa, Unknown
  source: /note=“Description of Unknown Furin protease recognition sequence”
LKRHQR

(19) United States
(12) Patent Application Publication
(10) Pub. No.: US 2021/0145889 A1
(43) Pub. Date: May 20, 2021

(54) METHODS, COMPOSITIONS, AND IMPLANTABLE ELEMENTS COMPRISING STEM CELLS

(71) Applicant: SIGILON THERAPEUTICS, INC., Cambridge, MA (US)

(72) Inventors: Devyn McKinley Smith, Barrington, RI (US); David Peritt, Skokie, IL (US); Omid Veiseh, Bellaire, TX (US); Richard Heidebrecht, Somerville, MA (US); Robert James Miller, East Bridgewater, MA (US)

(21) Appl. No.: 17/045,137

(22) PCT Filed: Mar. 27, 2019
(86) PCT No.: PCT/US2019/024385

§ 371 Date: Oct. 2, 2020

Related U.S. Application Data
(60) Provisional application No. 62/652,877, filed on Apr. 4, 2018.
(60) Provisional application No. 62/652,878, filed on Apr. 4, 2018.

(51) Int. Cl. A61K 35/28; A61K 9/00; A61K 47/22; A61K 9/50; A61L 27/38; A61K 38/37; A61K 38/48; C12N 5/00; A61K 38/28; C12N 5/0775

(52) U.S. Cl. CPC A61K 35/28; A61K 9/0024; A61K 47/22; A61K 9/5036; A61L 27/3834; C12N 5/0663; A61K 38/4846; C12Y 304/21022; C12N 5/0012; A61K 38/28; A61K 38/37

(57) ABSTRACT

Described herein are cell compositions comprising a mesenchymal stem function cell (MSFC), e.g., an engineered MSFC or derivatives thereof, as well as compositions, pharmaceutical products, and implantable elements comprising an MSFC, and methods of making and using the same. The cells and compositions may express a therapeutic agent useful for the treatment of a disease, disorder, or condition described herein.

### CLAIM OF PRIORITY

This application claims priority to U.S. Provisional Application No. 62/652,877, filed Apr. 4, 2018, and U.S. Application No. 62/652,878, filed Apr. 4, 2018. The disclosure of each of the foregoing applications is incorporated herein by reference in its entirety.

### BACKGROUND

The function of implanted cells, tissues, and devices depends on numerous factors including the ability to provide a product and the biological immune response pathway of the recipient (Anderson et al., Semin Immunol (2008) 20:86-100; Langer, Adv Mater (2009) 21:3235-3236). Selection of cells and the modulation of the immune response may impart a beneficial effect on the fidelity and function of implanted cells, tissues, and devices.

### SUMMARY

Described herein are cell compositions comprising a mesenchymal stem function cell (MSFC), e.g., an engineered MSFC or derivatives thereof, as well as compositions, pharmaceutical products, and implantable elements comprising an MSFC, and methods of making and using the same. In some embodiments, the MSFCs, compositions, and implantable elements described herein produce a therapeutic agent (such as a replacement agent) useful, e.g., for the treatment of a disease, disorder or condition in a subject, e.g., a blood clotting disorder or a lysosomal storage disease. In some embodiments, the compositions and implantable elements comprising an MSFC, e.g., an engineered MSFC, are capable of modulating the immune response or the effect of an immune response in a subject.

In one aspect, the present disclosure features an implantable element comprising an engineered MSFC that produces (e.g., or is capable of producing) a therapeutic agent. The therapeutic agent may be a biological substance, such as a nucleic acid (e.g., a nucleotide, DNA, or RNA), a polypeptide, a lipid, a sugar (e.g., a monosaccharide, disaccharide, oligosaccharide, or polysaccharide), or a small molecule. In some embodiments, the therapeutic agent is a replacement therapy or a replacement protein, e.g., useful for the treatment of a blood clotting disorder or a lysosomal storage disease in a subject.

In some embodiments, the implantable element comprises a single engineered MSFC. In some embodiments, the implantable element comprises a plurality of engineered MSFCs, e.g., provided as a cluster or disposed on a microcarrier. In some embodiments, the engineered MSFC or engineered MSFCs produce(s) or release(s) a therapeutic agent (e.g., a polypeptide) for at least 5 days, e.g., when implanted into a subject or when evaluated by a reference method, e.g., microscopy or Western blotting. In some embodiments, the implantable element comprises an encapsulating component (e.g., formed in situ on or surrounding an engineered MSFC, or preformed prior to combination with an engineered MSFC). In some embodiments, the implantable element is chemically modified, e.g., with a compound of Formula (I) or a salt thereof as described herein.

In another aspect, the present disclosure features a method of treating a subject comprising administering to the subject an implantable element comprising an MSFC (e.g., an engineered MSFC). In some embodiments, the implantable element comprises a plurality of MSFCs (e.g., a plurality of engineered MSFCs). In some embodiments, the subject is a human. In some embodiments, the MSFC (e.g., the engineered MSFC) is a human MSFC. In some embodiments, the implantable element comprises an MSFC (e.g., an engineered MSFC) that produces (e.g., or is capable of producing) a therapeutic agent, such as a nucleic acid (e.g., a nucleotide, DNA, or RNA), a polypeptide, a lipid, a sugar (e.g., a monosaccharide, disaccharide, oligosaccharide, or polysaccharide), or a small molecule. In some embodiments, the therapeutic agent is a replacement therapy or a replacement protein, e.g., useful for the treatment of a blood clotting disorder or a lysosomal storage disease in a subject. In some embodiments, the implantable element is formulated for implantation or injection into a subject. In some embodiments, the implantable element is administered to, implanted in, or provided to a site other than the central nervous system, brain, spinal column, eye, or retina. In some embodiments, the implantable element is administered to or implanted or injected in the peritoneal cavity (e.g., the lesser sac, also known as the omental bursa), the omentum, or the subcutaneous fat of a subject.

In another aspect, the present disclosure features a method of making or manufacturing an implantable element comprising an MSFC (e.g., an engineered MSFC). In some embodiments, the method comprises providing an MSFC (e.g., an engineered MSFC) and disposing the MSFC (e.g., the engineered MSFC) in an enclosing component, e.g., as described herein. In some embodiments, the implantable element comprises a plurality of MSFCs (e.g., a plurality of engineered MSFCs). In some embodiments, the implantable element comprises a plurality of MSFCs (e.g., a plurality of engineered MSFCs), e.g., provided as a cluster or disposed on a microcarrier. In some embodiments, the enclosing component is formed in situ on or surrounding an MSFC (e.g., an engineered MSFC), a plurality of MSFCs (e.g., a plurality of engineered MSFCs), or a microcarrier (e.g., a bead or matrix) comprising an MSFC or engineered MSFCs. In some embodiments, the enclosing component is preformed prior to combination with the enclosed MSFC (e.g., the engineered MSFC), a plurality of MSFCs (e.g., plurality of engineered MSFCs), or a microcarrier (e.g., a bead or matrix) comprising an MSFC or MSFCs. In some embodiments, the enclosing component comprises a flexible polymer (e.g., PLA, PLG, PEG, CMC, or a polysaccharide, e.g., alginate). In some embodiments, the enclosing component comprises an inflexible polymer or metal housing. In some embodiments, the enclosing component is chemically modified, e.g., with a compound of Formula (I) or a salt thereof described herein.

In another aspect, the present disclosure features a method of evaluating an implantable element comprising an engineered MSFC. In some embodiments, the method comprises providing an engineered MSFC and evaluating a structural or functional parameter of the encapsulated MSFC. In some embodiments, the method comprises evaluating the engineered MSFC or a plurality of engineered MSFCs for one or more of: a) viability; b) the production of a therapeutic agent (e.g., an engineered RNA or polypeptide); c) the uptake of a nutrient or oxygen; or d) the production of a waste product. In some embodiments, the evaluation is performed at least 1, 5, 10, 20, 30, or 60 days after formation of the implantable element or administration of the implantable element to a subject.

In another aspect, the present disclosure features a method of monitoring an implantable element comprising an engineered MSFC. In some embodiments, the method comprises obtaining, e.g., by testing the subject or a sample therefrom, the level of a parameter; and comparing, e.g., by testing the subject or a sample therefrom, the value obtained to that of a reference value. In some embodiments, the parameter comprises a) cell viability; b) level of production of a therapeutic agent (e.g., an engineered RNA or polypeptide); c) the uptake of a nutrient or oxygen; or d) the production of a waste product. In some embodiments, the evaluation is performed at least 1, 5, 10, 20, 30, or 60 days after formation of the implantable element or administration of the implantable element to a subject.

In another aspect, the present disclosure features a plurality of engineered MSFCs. In some embodiments, the plurality has a preselected form factor or a form factor described herein, e.g., a cluster of engineered MSFCs. In some embodiments, the cluster of engineered MSFCs comprises at least about 5, 10, 25, 50, 75, 100, 200, 250, 300, 400, 500, or more engineered MSFCs. In some embodiments, the cluster is globular or spherical. In some embodiments, the cluster is not a monolayer. In some embodiments, the cluster has a density of about 500 cells/cm² or more. In some embodiments, the plurality of engineered MSFCs is disposed on a microcarrier (e.g., a bead or matrix).

In another aspect, the present disclosure features a substrate comprising a plurality of chambers, wherein each chamber comprises an MSFC (e.g., an engineered MSFC). In some embodiments, each chamber comprises a plurality of MSFC (e.g., a plurality of engineered MSFCs). In some embodiments, the plurality comprises a cluster of MSFCs (e.g., engineered MSFCs) and/or is disposed on a microcarrier (e.g., a bead or matrix).

In another aspect, the present disclosure features a microcarrier, e.g., a bead or matrix, having disposed thereon an engineered MSFC.

In another aspect, the present disclosure features a preparation of engineered MSFCs, wherein the preparation comprises at least about 10,000 engineered MSFCs, e.g., at least about 15,000; 20,000; 25,000; 30,000; 35,000; 40,000; 50,000; 60,000; 70,000; 80,000; 90,000; 100,000 or more engineered MSFCs.

The details of one or more embodiments of the disclosure are set forth herein. Other features, objects, and advantages of the disclosure will be apparent from the Detailed Description, the Figures, the Examples, and the Claims.

### BRIEF DESCRIPTION OF THE DRAWINGS

FIG. 1 shows the amino acid sequence of the Factor VIII-BDD encoded by an exemplary engineered MSFC (SEQ ID NO: 1), with the signal sequence underlined.

FIG. 2 shows the amino acid sequence of a human wild type Factor IX protein (SEQ ID NO. 2).

FIG. 3 shows in Table 2-3 exemplary amino acid sequences for therapeutic polypeptides and useful for engineering MSFCs.

### DETAILED DESCRIPTION

The present disclosure features cell therapy compositions comprising mesenchymal stem function cells (MSFCs), e.g., engineered MSFCs or cell derivatives thereof, as well as compositions thereof and implantable elements comprising the same. In some embodiments, the MSFCs, compositions, and implantable elements are useful for the prevention or treatment of a disease, disorder, or condition. The MSFCs described herein exhibit advantageous properties, for example, they are not in a terminal state of differentiation and can terminally differentiate into one or more cell types. In some embodiments, the MSFCs are engineered to produce a therapeutic agent (e.g., a therapeutic polypeptide) and are encapsulated by a material and/or present within an implantable element suitable for administration to a subject.

### Definitions

The following terms are intended to have the meanings presented therewith below and are useful in understanding the description and intended scope of the present disclosure.

“Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., fluorescence microscope to acquire fluorescence microscopy data.

“Mesenchymal stem function cell” or “MSFC,” as those terms are used herein, refers to a cell derived from, or having at least one characteristic specific to a cell of, mesodermal lineage, and wherein the MSFC is i) not in a terminal state of differentiation and ii) can terminally differentiate into one or more cell types. An MSFC does not comprise a cell of endodermal origin, e.g., a gut cell, or of ectodermal origin, e.g., a cell derived from skin, CNS, or a neural cell.

In an embodiment, the MSFC is multipotent. In an embodiment the MSFC is not totipotent. In an embodiment, an MSFC comprises one or more of the following characteristics:

a) it comprises a mesenchymal stem cell (MSC) or a cell derived therefrom, including a cell derived from a primary cell culture of MSCs, a cell isolated directly (without long term culturing, e.g., less than 5 or 10 passages or rounds of cell division since isolation) from naturally occurring MSCs, e.g., from a human or other mammal, a cell derived from a transformed, a pluripotent, an immortalized, or a long term (e.g., more than 5 or 10 passages or rounds of cell division) MSC culture. In an embodiment, the MSFC is derived from a human source, e.g., the blood (e.g., peripheral blood), bone marrow (e.g., the iliac crest, femora, tibiae, spine, rib, or knee), synovial tissue, adipose tissue, skin, fetal tissue, umbilical cord, or the placenta;

b) it comprises a cell that has been obtained from a less differentiated cell, e.g., a cell developed, programmed, or reprogramed (e.g., in vitro) into an MSC or a cell that is, except for any genetic engineering, substantially similar to one or more of a naturally occurring MSC or a cell from a primary or long term culture of MSCs, or a cell described in a) above. Examples of less differentiated cells from which MSFC can be derived include IPS cells, embryonic stem cells, or other totipotent or pluripotent cells; see, e.g., Chen, Y. S. et al (2012) Stem Cells Transl Med 1(83-95); Frobel, J et al (2014) Stem Cell Reports 3(3):414-422; Zou, L et al (2013) Sci Rep 3:2243;

c) it is multipotent, e.g., as measured by any assay capable of providing information about cell multipotency, e.g., microscopy;

d) it exhibits a characteristic mononuclear ovoid, stellate shape or spindle shape, with a round to oval nucleus. The oval elongate nucleus may have prominent nucleoli and a mix of heterochromatin and euchromatin. An MSFC (e.g., an MSC) may have little cytoplasm, but many thin processes that appear to extend from the nucleus;

e) it is capable of cell division, e.g., as measured any assay capable of providing information about cell division, e.g., microscopy. In an embodiment, an MSFC is capable of cell division in culture (e.g., prior to being encapsulated or incorporated into a device). In an embodiment, it is capable of cell division after being encapsulated, e.g., encapsulated as described herein, or incorporated into a device (e.g., a device described herein). In an embodiment, it is incapable of cell division after reaching confluence;

f) it is capable of differentiating into a mesenchymal cell lineage, e.g., an osteoblast, a chrondoblast, an adipocyte, or a fibroblast;

g) it expresses a mesenchymal cell marker, e.g., one, two, three, four, five or all of CD105, CD106, CD73, CD90, Stro-1, CD49a, CD29, CD44, CD146, CD166, TNAP+, THY-1+, Stro-2, Stro-4, and alkaline phosphatase;

h) it does not express significant levels of one, two, three, or any of CD34, CD31, VE-cadherin, CD45, HLA-DR, CD11b and a glycophorin or leukocyte differentiation antigen, e,g, CD14, CD33, CD3 and CD19;

i) it expresses one, two, or all of CD75, CD90, and CD105 and does not express one, two, or any of CD45, CD34, and CD14;

j) it is anti-inflammatory or immune dampening, e.g., as measured by any method capable of providing information regarding inflammation, e.g., in vivo inhibition of T cell proliferation;

k) it is capable of being adherent, e.g., plastic adherent, e.g., as determined by, e.g., visual inspection; or

l) can grow in three dimensions, e.g., as determined by, e.g., visual inspection.

In an embodiment, the MSFC comprises one of properties a-1. In an embodiment, the MSFC comprises two of properties a-1. In an embodiment, the MSFC comprises three of properties a-1. In an embodiment, the MSFC comprises four of properties a-1. In an embodiment, the MSFC comprises five of properties a-1. In an embodiment, the MSFC comprises six of properties a-1. In an embodiment, the MSFC comprises seven of properties a-1. In an embodiment, the MSFC comprises eight of properties a-1.

In an embodiment, the MSFC comprises properties a-e. In an embodiment, the MSFC comprises property f. In an embodiment, the MSFC comprises property g. In an embodiment, the MSFC comprises property h. In an embodiment, the MSFC comprises property j.

In some embodiments, an MSFC, including an engineered MSFC, is not an islet cell. An islet cell as defined herein is a cell that comprises any naturally occurring or any synthetically created, or modified, cell that is intended to recapitulate, mimic or otherwise express, in part or in whole, the functions, in part or in whole, of the cells of the pancreatic islets of Langerhans. An MSFC, including an engineered MSFC, is not capable of producing insulin (e.g., insulin A-chain, insulin B-chain, or proinsulin), e.g., in an amount effective to treat diabetes or another disease or condition that may be treated with insulin. In some embodiments, an MSFC is not capable of producing insulin in a glucose-responsive manner. An MSFC, including an engineered MSFC, is not an induced pluripotent cell that is engineered into a differentiated insulin-producing pancreatic beta cell.

“Administer,” “administering,” or “administration,” as used herein, refer to implanting, absorbing, ingesting, injecting, or otherwise introducing an entity (e.g., an MSFC, e.g., an engineered MSFC, or a composition thereof, or an implantable element comprising an MSFC, an engineered MSFC, or a composition thereof), or providing the same to a subject.

“Cell,” as used herein, refers to an engineered cell, e.g., an engineered MSFC, or a cell that is not engineered, e.g., a non-engineered MSFC.

“Conservatively modified variants” or “conservative substitution”, as used herein, refers to a variant of a reference peptide or polypeptide that is identical to the reference molecule, except for having one or more conservative amino acid substitutions in its amino acid sequence. In an embodiment, a conservatively modified variant consists of an amino acid sequence that is at least 70%, 80%, 85%, 90%, 95%, 97%, 98% or 99% identical to the reference amino acid sequence. A conservative amino acid substitution refers to substitution of an amino acid with an amino acid having similar characteristics (e.g., charge, side-chain size, hydrophobicity/hydrophilicity, backbone conformation and rigidity, etc.) and which has minimal impact on the biological activity of the resulting substituted peptide or polypeptide. Conservative substitution tables of functionally similar amino acids are well known in the art, and exemplary substitutions grouped by functional features are set forth in Amino Acid Table 1 below.

AMINO ACID TABLE 1
Exemplary conservative amino acid substitution groups.

| Feature | Conservative Amino Group |
|---|---|
| Charge/Polarity | His, Arg, Lys |
| | Asp, Glu |
| | Cys, Thr, Ser, Gly, Asn, Gln, Tyr |
| | Ala, Pro, Met, Leu, Ile, Val, Phe, Trp |
| Hydrophobicity | Asp, Glu, Asn, Gln, Arg, Lys |
| | Cys, Ser, Thr, Pro, Gly, His, Tyr |
| | Ala, Met, Ile Leu, Val, Phe, Trp |
| Structural/Surface Exposure | Asp, Glu, Asn, Aln, His, Arg, Lys |
| | Cys, Ser, Tyr, Pro, Ala, Gly, Trp, Tyr |
| | Met, Ile, Leu, Val, Phe |
| Secondary Structure Propensity | Ala, Glu, Aln, His, Lys, Met, Leu, Arg |
| | Cys, Thr, Ile, Val, Phe, Tyr, Trp |
| | Ser, Gly, Pro, Asp, Asn |
| Evolutionary Conservation | Asp, Glu |
| | His, Lys, Arg |
| | Asn, Gln |
| | Ser, Thr |
| | Leu, Ile, Val |
| | Phe, Tyr, Trp |
| | Ala, Gly |
| | Met, Cys |

“Consists essentially of”, and variations such as “consist essentially of” or “consisting essentially of” as used throughout the specification and claims, indicate the inclusion of any recited elements or group of elements, and the optional inclusion of other elements, of similar or different nature than the recited elements, that do not materially change the basic or novel properties of the specified molecule, composition, device, or method. As a non-limiting example, a therapeutic protein that consists essentially of a recited amino acid sequence may also include one or more amino acids, including additions at the N-terminus, C-terminus or within the recited amino acid sequence, of one or more amino acid residues, which do not materially affect the relevant biological activity of the therapeutic protein, respectively. As another non-limiting example, a promoter that consists essentially of a recited nucleotide sequence may contain one or more additional nucleotides that do not materially change the relevant biological activity of the promoter, e.g. the amount of transcription of an operably linked coding sequence, e.g., as determined by quantifying corresponding RNA or protein levels.

“Effective amount” as used herein refers to an amount of a composition of MSFCs, e.g., engineered MSFCs, or an agent, e.g., a therapeutic agent, produced by an MSFC, e.g., an engineered MSFC, sufficient to elicit a biological response, e.g., to treat a disease, disorder, or condition. As will be appreciated by those of ordinary skill in this art, the effective amount may vary depending on such factors as the desired biological endpoint, the pharmacokinetics of the therapeutic agent, composition or implantable element, the condition being treated, the mode of administration, and the age and health of the subject. An effective amount encompasses therapeutic and prophylactic treatment. For example, to treat a fibrotic condition, an effective amount of a compound may reduce the fibrosis or stop the growth or spread of fibrotic tissue.

An “endogenous nucleic acid” as used herein, is a nucleic acid that occurs naturally in a subject cell.

An “endogenous polypeptide,” as used herein, is a n polypeptide that occurs naturally in a subject cell.

“Engineered cell,” as used herein, is a cell, e.g., an MSFC, having a non-naturally occurring alteration, and typically comprises a nucleic acid sequence (e.g., DNA or RNA) or a polypeptide not present (or present at a different level than) in an otherwise similar cell under similar conditions that is not engineered (an exogenous nucleic acid sequence). In an embodiment, an engineered cell comprises an exogenous nucleic acid (e.g., a vector or an altered chromosomal sequence). In an embodiment, an engineered cell comprises an exogenous polypeptide. In an embodiment, an engineered cell comprises an exogenous nucleic acid sequence, e.g., a sequence, e.g., DNA or RNA, not present in a similar cell that is not engineered. In an embodiment, the exogenous nucleic acid sequence is chromosomal, e.g., the exogenous nucleic acid sequence is an exogenous sequence disposed in endogenous chromosomal sequence. In an embodiment, the exogenous nucleic acid sequence is chromosomal or extra chromosomal, e.g., a non-integrated vector. In an embodiment, the exogenous nucleic acid sequence comprises an RNA sequence, e.g., an mRNA. In an embodiment, the exogenous nucleic acid sequence comprises a chromosomal or extra-chromosomal exogenous nucleic acid sequence that comprises a sequence which is expressed as RNA, e.g., mRNA or a regulatory RNA. In an embodiment, the exogenous nucleic acid sequence comprises a chromosomal or extra-chromosomal nucleic acid sequence that comprises a sequence which encodes a polypeptide or which is expressed as a polypeptide. In an embodiment, the exogenous nucleic acid sequence comprises a first chromosomal or extra-chromosomal exogenous nucleic acid sequence that modulates the conformation or expression of a second nucleic acid sequence, wherein the second amino acid sequence can be exogenous or endogenous. For example, an engineered cell can comprise an exogenous nucleic acid that controls the expression of an endogenous sequence. In an embodiment, an engineered cell comprises a polypeptide present at a level or distribution which differs from the level found in a similar cell that has not been engineered. In an embodiment, an engineered cell comprises an MSFC engineered to provide an RNA or a polypeptide. For example, an engineered cell (e.g., an engineered MSFC) may comprise an exogenous nucleic acid sequence comprising a chromosomal or extra-chromosomal exogenous nucleic acid sequence that comprises a sequence which is expressed as RNA, e.g., mRNA or a regulatory RNA. In an embodiment, an engineered cell (e.g., an MSFC) comprises an exogenous nucleic acid sequence that comprises a chromosomal or extra-chromosomal nucleic acid sequence comprising a sequence which encodes a polypeptide or which is expressed as a polypeptide. In an embodiment, an engineered cell (e.g., an MSFC) comprises an exogenous nucleic acid sequence that modulates the conformation or expression of an endogenous sequence.

An “exogenous nucleic acid,” as used herein, is a nucleic acid that does not occur naturally in a subject cell.

An “exogenous polypeptide,” as used herein, is polypeptide that does not occur naturally in a subject cell.

“Factor VII protein” or “FVII protein” as used herein, means a polypeptide that comprises the amino acid sequence of a naturally-occurring factor VII protein or variant thereof that has a FVII biological activity, e.g., promoting blood clotting, as determined by an art-recognized assay, unless otherwise specified. Naturally-occurring FVII exists as a single chain zymogen, a zymogen-like two-chain polypeptide and a fully activated two-chain form (FVIIa). In some embodiments, reference to FVII includes single-chain and two-chain forms thereof, including zymogen-like and FVIIa. FVII proteins that may be expressed by MSFCs described herein, e.g., engineered MSFCs, include wild-type primate (e.g., human), porcine, canine, and murine proteins, as well as variants of such wild-type proteins, including fragments, mutants, variants with one or more amino acid substitutions and/or deletions. In some embodiments, a variant FVII protein is capable of being activated to the fully activated two-chain form (Factor VIIa) that has at least 50%, 75%, 90% or more (including >100%) of the activity of wild-type Factor VIIa. Variants of FVII and FVIIa are known, e.g., marzeptacog alfa (activated) (MarzAA) and the variants described in European Patent No. 1373493, U.S. Pat. Nos. 7,771,996, 9,476,037 and US Patent Publication No. US20080058255.

Factor VII biological activity may be quantified by an art recognized assay, unless otherwise specified. For example, FVII biological activity in a sample of a biological fluid, e.g., plasma, may be quantified by (i) measuring the amount of Factor Xa produced in a system comprising TF embedded in a lipid membrane and Factor X. (Persson et al., J. Biol. Chem. 272:19919-19924, 1997); (ii) measuring Factor X hydrolysis in an aqueous system; (iii) measuring its physical binding to TF using an instrument based on surface plasmon resonance (Persson, FEBS Letts. 413:359-363, 1997); or (iv) measuring hydrolysis of a synthetic substrate; and/or (v) measuring generation of thrombin in a TF-independent in vitro system. In an embodiment, FVII activity is assessed by a commercially available chromogenic assay (BIOPHEN FVII, HYPHEN BioMed Neuville sur Oise, France), in which the biological sample containing FVII is mixed with thromboplastin calcium, Factor X and SXa-11 (a chromogenic substrate specific for Factor Xa.

“Factor VIII protein” or “FVIII protein” as used herein, means a polypeptide that comprises the amino acid sequence of a naturally-occurring factor VIII polypeptide or variant thereof that has an FVIII biological activity, e.g., coagulation activity, as determined by an art-recognized assay, unless otherwise specified. FVIII proteins that may be expressed by MSFCs described herein, e.g., engineered MSFCs, include wild-type primate (e.g., human), porcine, canine, and murine proteins, as well as variants of such wild-type proteins, including fragments, mutants, variants with one or more amino acid substitutions and/or deletions, B-domain deletion (BDD) variants, single chain variants and fusions of any of the foregoing wild-type or variants with a half-life extending polypeptide. In an embodiment, the MSFCs are engineered to encode a precursor factor VIII polypeptide (e.g., with the signal sequence) with a full or partial deletion of the B domain. In an embodiment, the MSFCs are engineered to encode a single chain factor VIII polypeptide which contains A variant FVIII protein preferably has at least 50%, 75%, 90% or more (including >100%) of the coagulation activity of the corresponding wild-type factor VIII. Assays for measuring the coagulation activity of FVIII proteins include the one stage or two stage coagulation assay (Rizza et al., 1982, Coagulation assay of FVIII:C and FIXa in Bloom ed. The Hemophelias. NY Churchill Livingston 1992) or the chromogenic substrate FVIII:C assay (Rosen, S. 1984. Scand J Haematol 33:139-145, suppl.)

A number of FVIII-BDD variants are known, and include, e.g., variants with the full or partial B-domain deletions disclosed in any of the following U.S. Pat. No. 4,868,112 (e.g., col. 2, line 2 to col. 19, line 21 and table 2); U.S. Pat. No. 5,112,950 (e.g., col. 2, lines 55-68, FIG. 2, and example 1); U.S. Pat. No. 5,171,844 (e.g., col. 4, line 1 22 to col. 5, line 36); U.S. Pat. No. 5,543,502 (e.g., col. 2, lines 17-46); U.S. Pat. Nos. 5,595,886; 5,610,278; 5,789,203 (e.g., col. 2, lines 26-51 and examples 5-8); U.S. Pat. No. 5,972,885 (e.g., col. 1, lines 25 to col. 2, line 40); U.S. Pat. No. 6,048,720 (e.g., col. 6, lines 1-22 and example 1); U.S. Pat. Nos. 6,060,447; 6,228,620; 6,316,226 (e.g., col. 4, line 4 to col. 5, line 28 and examples 1-5); U.S. Pat. Nos. 6,346,513; 6,458,563 (e.g., col. 4, lines 25-53) and U.S. Pat. No. 7,041,635 (e.g., col. 2, line 1 to col. 3, line 19, col. 3, line 40 to col. 4, line 67, col. 7, line 43 to col. 8, line 26, and col. 11, line 5 to col. 13, line 39).

In some embodiments, a FVIII-BDD protein expressed by engineered MSFCs has one or more of the following deletions of amino acids in the B-domain: (i) most of the B domain except for amino-terminal B-domain sequences essential for intracellular processing of the primary translation product into two polypeptide chains (WO 91/09122); (ii) a deletion of amino acids 747-1638 (Hoeben R. C., et al. J. Biol. Chem. 265 (13): 7318-7323 (1990)); amino acids 771-1666 or amino acids 868-1562 (Meulien P., et al. Protein Eng. 2(4):301-6 (1988); amino acids 982-1562 or 760-1639 (Toole et al., Proc. Natl. Acad. Sci. U.S.A. 83:5939-5942 (1986)); amino acids 797-1562 (Eaton et al., Biochemistry 25:8343-8347 (1986)); 741-1646 (Kaufman, WO 87/04187)), 747-1560 (Sarver et al., DNA 6:553-564 (1987)); amino acids 741-1648 (Pasek, WO 88/00831)), amino acids 816-1598 or 741-1689 (Lagner (Behring Inst. Mitt. (1988) No 82:16-25, EP 295597); a deletion that includes one or more residues in a furin protease recognition sequence, e.g., LKRHQR at amino acids 1643-1648, including any of the specific deletions recited in U.S. Pat. No. 9,956,269 at col. 10, line 65 to col. 11, line 36.

In other embodiments, a FVIII-BDD protein retains any of the following B-domain amino acids or amino acid sequences: (i) one or more N-linked glycosylation sites in the B-domain, e.g., residues 757, 784, 828, 900, 963, or optionally 943, first 226 amino acids or first 163 amino acids (Miao, H. Z., et al., Blood 103(a): 3412-3419 (2004), Kasuda, A., et al., J. Thromb. Haemost. 6: 1352-1359 (2008), and Pipe, S. W., et al., J. Thromb. Haemost. 9: 2235-2242 (2011).

In some embodiments, the FVIII-BDD protein is a single-chain variant generated by substitution of one or more amino acids in the furin protease recognition sequence (LKRHQR at amino acids 1643-1648) that prevents proteolytic cleavage at this site, including any of the substitutions at the R1645 and/or R1648 positions described in U.S. Pat. Nos. 10,023,628, 9,394,353 and 9,670,267.

In some embodiments, any of the above FVIII-BDD proteins may further comprise one or more of the following variations: a F309S substitution to improve expression of the FVIII-BDD protein (Miao, H. Z., et al., Blood 103(a): 3412-3419 (2004); albumin fusions (WO 2011/020866); and Fc fusions (WO 04/101740).

All FVIII-BDD amino acid positions referenced herein refer to the positions in full-length human FVIII, unless otherwise specified.

“Factor IX protein” or “FIX protein”, as used herein, means a polypeptide that comprises the amino acid sequence of a naturally-occurring factor IX protein or variant thereof that has a FIX biological activity, e.g., coagulation activity, as determined by an art-recognized assay, unless otherwise specified. FIX is produced as an inactive zymogen, which is converted to an active form by factor XIa excision of the activation peptide to produce a heavy chain and a light chain held together by one or more disulfide bonds. FIX proteins that may be expressed by MSFCs described herein (e.g., engineered MSFCs) include wild-type primate (e.g., human), porcine, canine, and murine proteins, as well as variants of such wild-type proteins, including fragments, mutants, variants with one or more amino acid substitutions and/or deletions and fusions of any of the foregoing wild-type or variant proteins with a half-life extending polypeptide. In an embodiment, MSFCs are engineered to encode a full-length wild-type human factor IX polypeptide (e.g., with the signal sequence) or a functional variant thereof. A variant FIX protein preferably has at least 50%, 75%, 90% or more (including >100%) of the coagulation activity of wild-type factor VIX. Assays for measuring the coagulation activity of FIX proteins include the Biophen Factor IX assay (Hyphen BioMed) and the one stage clotting assay (activated partial thromboplastin time (aPTT), e.g., as described in EP 2032607, thrombin generation time assay (TGA) and rotational thromboelastometry, e.g., as described in WO 2012/006624.

A number of functional FIX variants are known and may be expressed by MSFCs of the present disclosure, including any of the functional FIX variants described in the following international patent publications: WO 02/040544 A3 at page 4, lines 9-30 and page 15, lines 6-31; WO 03/020764 A2 in Tables 2 and 3 at pages 14-24, and at page 12, lines 1-27; WO 2007/149406 A2 at page 4, line 1 to page 19, line 11; WO 2007/149406 A2 at page 19, line 12 to page 20, line 9; WO 08/118507 A2 at page 5, line 14 to page 6, line 5; WO 09/051717 A2 at page 9, line 11 to page 20, line 2; WO 09/137254 A2 at page 2, paragraph [006] to page 5, paragraph [011] and page 16, paragraph [044] to page 24, paragraph [057]; WO 09/130198 A2 at page 4, line 26 to page 12, line 6; WO 09/140015 A2 at page 11, paragraph [0043] to page 13, paragraph [0053]; WO 2012/006624; WO 2015/086406.

In certain embodiments, the FIX polypeptide comprises a wild-type or variant sequence fused to a heterologous polypeptide or non-polypeptide moiety extending the half-life of the FIX protein. Exemplary half-life extending moieties include Fc, albumin, a PAS sequence, transferrin, CTP (28 amino acid C-terminal peptide (CTP) of human chorionic gonadotropin (hCG) with its 4 O-glycans), polyethylene glycol (PEG), hydroxyethyl starch (HES), albumin binding polypeptide, albumin-binding small molecules, or any combination thereof. An exemplary FIX polypeptide is the rFIXFc protein described in WO 2012/006624, which is an FIXFc single chain (FIXF c-sc) and an Fc single chain (Fc-sc) bound together through two disulfide bonds in the hinge region of Fc.

FIX variants also include gain and loss of function variants. An example of a gain of function variant is the “Padua” variant of human FIX, which has a L (leucine) at position 338 of the mature protein instead of an R (arginine) (corresponding to amino acid position 384 of SEQ ID NO:2), and has greater catalytic and coagulant activity compared to wild-type human FIX (Chang et al., J. Biol. Chem., 273:12089-94 (1998)). An example of a loss of function variant is an alanine substituted for lysine in the fifth amino acid position from the beginning of the mature protein, which results in a protein with reduced binding to collagen IV (e.g., loss of function).

“Form factor,” as used herein, refers to one or more of: the number of MSFCs present in a plurality of MSFCs, the shape of the plurality of MSFCs, the level of contact between the MSFCs of the plurality, or the level of junctions formed between the MSFCs of the plurality. In an embodiment, the plurality of MSFCs are provided as a cluster, other aggregation, or other plurality having preselected values (or values described herein) for one or more or all of parameter relating to size, shape, shared contact with one another, or number of junctions between one another. For example, in an embodiment, the MSFCs of the plurality have an average minimum number of junctions per MSFC, e.g., as evaluated by fixation or microscopy. In an embodiment, the MSFCs can exhibit the form factor at one or more or all of: prior to, during, or after administration or provision to a subject. In an embodiment, the MSFCs can exhibit the form factor at one or more or all of: prior to, during, or after administration or provision to a subject. Exemplary form factors include monolayers of MSFCs, clusters of MSFCs, or disposition on a microcarrier (e.g., a bead or matrix).

“Interleukin 2 protein” or “IL-2 protein”, as used herein means a polypeptide comprising the amino acid sequence of a naturally-occurring IL-2 protein or variant thereof that has an IL-2 biological activity, e.g., activate IL-2 receptor signaling in Treg cells, as determined by an art-recognized assay, unless otherwise specified. IL-2 proteins that may be expressed by MSFCs described herein, e.g., engineered MSFCs, include wild-type primate (e.g., human), porcine, canine, and murine proteins, as well as variants of such wild-type proteins. A variant IL-2 protein preferably has at least 50%, 75%, 90% or more (including >100%) of the biological activity of the corresponding wild-type IL-2. Biological activity assays for IL-2 proteins are described in U.S. Pat. No. 10,035,836, and include, e.g., measuring the levels of phosphorylated STATS protein in Treg cells compared to CD4+CD25-/low T cells or NK cells. Variant IL-2 proteins that may be produced by MSFCs of the present disclosure (e.g., engineered MSFCs) include proteins with one or more of the following amino acid substitutions: N88R, N88I, N88G, D20H, Q126L, Q126F, and C125S or C125A.

An “implantable element” as used herein, comprises an MSFC, e.g., a plurality of MSFCs, e.g., a cluster of MSFCs, wherein the MSFC or MSFCs are entirely or partially disposed within an enclosing component (which enclosing component is other than an MSFC), e.g., the enclosing component comprises a non-cellular component. In an embodiment, the enclosing component inhibits an immune attack, or the effect of the immune attack, on the enclosed MSFC or MSFCs. In an embodiment, the enclosing component comprises a semipermeable membrane or a semipermeable polymer matrix or coating. Typically, the enclosing component allows passage of small molecules, e.g., nutrients and waste products. Typically, the enclosing component allows passage of a therapeutic product (e.g., a therapeutic polypeptide) released by an MSFC disposed within the enclosing component. In an embodiment, placement of an implantable element within an enclosing component minimizes an effect of an immune response, e.g., a fibrotic response, of the subject directed at the implantable element, e.g., against an MSFC within an implantable element, e.g., as compared with a similar MSFC that is not disposed in an implantable element. In an embodiment, the enclosing component comprises a moiety, e.g., a moiety described herein, that minimizes an effect of an immune response, e.g., a fibrotic response, of the subject directed at the implantable element, e.g., against the enclosing component or an MSFC within the implantable element, e.g., as compared with a similar implantable element lacking the moiety. In an embodiment, the enclosing component comprises a moiety, e.g., a moiety described herein (e.g., a compound in Compound Table 1), that minimizes an effect of an immune response, e.g., a fibrotic response, of the subject directed at the implantable element, e.g., against the enclosing component or an MSFC within the implantable element, e.g., as compared with a similar implantable element lacking the moiety. In some embodiments, the enclosing component comprises a polymer hydrogel. In some embodiments, the polymer hydrogel comprises an alginate chemically modified with a compound in Compound Table 1; in an embodiment, the alginate has a molecular weight of <75 kDa. In an embodiment, the enclosing component is a hydrogel capsule which comprises a mixture of a chemically modified alginate and an unmodified alginate; in an embodiment, the unmodified alginate has a molecular weight of 150 kDa-250 kDa. In an embodiment, the G:M ratio of the alginate in each of the chemically modified and unmodified alginate is >1.

In an embodiment, an implantable element comprises an enclosing component that is formed, or could be formed, in situ on or surrounding an MSFC, e.g., a plurality of MSFCs, e.g., a cluster of MSFCs, or on a microcarrier, e.g., a bead, or a matrix comprising an MSFC or MSFCs (referred to herein as an “in-situ encapsulated implantable element”).

In an embodiment, the implantable element comprises an enclosing component that comprises a flexible polymer, e.g., alginate (e.g., a chemically modified alginate), PLA, PLG, PEG, CMC, or mixtures thereof (referred to herein as a “polymer encapsulated implantable device”).

In-situ encapsulated implantable devices and polymer encapsulated implantable devices (which categories are not mutually exclusive) are collectively referred to herein as encapsulated implantable elements.

An exemplary encapsulated implantable element comprises an MSFC, e.g., a plurality of MSFCs, e.g., a cluster of MSFCs, or a microcarrier, e.g., a bead, or a matrix comprising an MSFC or MSFCs, and an enclosing element comprising a coating of derivatized alginate. In some embodiments, an encapsulated implantable element has a largest linear dimension of no more than about 1.5 mm, 2 mm, 3 mm, 4 mm, 5 mm 6 mm, 7 mm, or 8 mm.

In an embodiment, an implantable element comprises an enclosing component that is preformed prior to combination with the enclosed MSFC, e.g., a plurality of MSFCs, e.g., a cluster of MSFCs, or a microcarrier, e.g., a bead or a matrix comprising an MSFC (referred to herein as device-based-implantable element, or DB-implantable element). In an embodiment a device-implantable element comprises an enclosing component that comprises a polymer or metal. An exemplary device-implantable element comprises an MSFC, e.g., a plurality of MSFCs, e.g., a cluster of MSFCs, or a microcarrier, e.g., a bead comprising an MSFC or MSFCs, disposed within an enclosing component comprising a preformed housing, e.g., an inflexible polymeric or metal housing or a flexible housing, e.g., a semipermeable membrane. In embodiments, a device-implantable element has a largest linear dimension of at least 1.5 mm, 2 mm, 3 mm, 4 mm, 5 mm 6 mm, 7 mm, or 8 mm.

“Parathyroid hormone protein” or “PTH protein” as used herein means a polypeptide that comprises the amino acid sequence of a naturally-occurring parathyroid hormone polypeptide or variant thereof that has a PTH biological activity, e.g., as determined by an art recognized assay. PTH polypeptides that may be expressed by MSFCs described herein (e.g., engineered MSFCs) include wild-type primate (e.g., human), porcine, canine, and murine polypeptides, as well as variants of such wild-type polypeptides. Such PTH polypeptides may consist essentially of the wild-type human sequence for pre-pro-PTH polypeptide (115 amino acids), pro-PTH polypeptide (90 amino acids), the mature 84-amino acid peptide (PTH(1-84)), and biologically active variants thereof, such as the truncated variant peptide PTH(1-34). PTH peptide variants with one or more amino acid substitutions in the human wild-type sequence have been described, e.g., in U.S. Pat. Nos. 7,410,948 and 8,563,513 and in US Patent Publication No. US20130217630. A PTH variant preferably has at least 50%, 75%, 90% or more (including >100%) of a biological activity of the corresponding wild-type PTH. An assay to detect certain PTH variants by tandem mass spectrometry is described in U.S. Pat. No. 8,383,417. A biological activity assay for PTH peptide variants—stimulation of adenylate cyclase as determined by measuring cAMP levels—is described in U.S. Pat. No. 7,410,948.

“Polypeptide”, as used herein, refers to a polymer comprising amino acid residues linked through peptide bonds and having at least two, and in embodiments, at least 10, 100, or 200 amino acid residues.

“Prevention,” “prevent,” and “preventing” as used herein refers to a treatment that comprises administering or applying a therapy, e.g., administering an MSFC, e.g., an engineered MSFC (e.g., as described herein), prior to the onset of a disease, disorder, or condition in order to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

A “replacement therapy” or “replacement protein” is a therapeutic protein or functional fragment thereof that replaces or augments a protein that is diminished, present in insufficient quantity, altered (e.g., mutated) or lacking in a subject having a disease or condition related to the diminished, altered or lacking protein. Examples are certain blood clotting factors in certain blood clotting disorders or certain lysosomal enzymes in certain lysosomal storage diseases. In an embodiment, a replacement therapy or replacement protein provides the function of an endogenous protein. In an embodiment, a or replacement therapy or replacement protein has the same amino acid sequence of a naturally occurring variant, e.g., a wild type allele or an allele not associated with a disorder, of the replaced protein. In an embodiment, or replacement therapy or a replacement protein differs in amino acid sequence from a naturally occurring variant, e.g, a wild type allele or an allele not associated with a disorder, e.g, the allele carried by a subject, at no more than about 1, 2, 3, 4, 5, 10, 15 or 20% of the amino acid residues.

“Sequence identity” or “percent identical”, when used herein to refer to two nucleotide sequences or two amino acid sequences, means the two sequences are the same within a specified region, or have the same nucleotides or amino acids at a specified percentage of nucleotide or amino acid positions within the specified when the two sequences are compared and aligned for maximum correspondence over a comparison window or designated region. Sequence identity may be determined using standard techniques known in the art including, but not limited to, any of the algorithms described in US Patent Publication No. 2017/02334455. In an embodiment, the specified percentage of identical nucleotide or amino acid positions is at least about 80%, 85%, 90%, 91%, 92%, 93%, 94%, 95%, 96%, 97%, 98%, 99% or higher.

“Subject” as used herein refers to a human or non-human animal. In an embodiment, the subject is a human (i.e., a male or female, e.g., of any age group, a pediatric subject (e.g., infant, child, adolescent) or adult subject (e.g., young adult, middle-aged adult, or senior adult)). In an embodiment, the subject is a non-human animal, for example, a mammal (e.g., a primate (e.g., a cynomolgus monkey or a rhesus monkey)). In an embodiment, the subject is a commercially relevant mammal (e.g., a cattle, pig, horse, sheep, goat, cat, or dog) or a bird (e.g., a commercially relevant bird such as a chicken, duck, goose, or turkey). In certain embodiments, the animal is a mammal. The animal may be a male or female and at any stage of development. A non-human animal may be a transgenic animal.

“Transcription unit” means a DNA sequence, e.g., present in an exogenous nucleic acid, that comprises at least a promoter sequence operably linked to a coding sequence, and may also comprise one or more additional elements that control or enhance transcription of the coding sequence into RNA molecules or translation of the RNA molecules into polypeptide molecules. In some embodiments, a transcription unit also comprises polyadenylation (polyA) signal sequence and polyA site. In an embodiment, a transcription unit is present in an exogenous, extra-chromosomal expression vector, or is present as an exogenous sequence integrated in a chromosome of an engineered MSFC described herein.

“Treatment,” “treat,” and “treating” as used herein refers to one or more of reducing, reversing, alleviating, delaying the onset of, or inhibiting the progress of one or more of a symptom, manifestation, or underlying cause, of a disease, disorder, or condition. In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. In some embodiments, treatment comprises prevention and in other embodiments it does not.

“Von Willebrand Factor protein” or “vWF protein”, as used herein, means a polypeptide that comprises the amino acid sequence of a naturally-occurring vWF polypeptide or variant thereof that has vWF biological activity, e.g., FVIII binding activity, as determined by an art-recognized assay, unless otherwise specified. vWF proteins that may be expressed by engineered MSFCs described herein include wild-type primate (e.g., human), porcine, canine, and murine proteins, as well as variants of such wild-type proteins. The MSFCs may be engineered to encode any of the following vWF polypeptides: precursor vWF of 2813 amino acids, a vWF lacking the signal peptide of 22 amino acids and optionally the prepropeptide of 741 amino acids, mature vWF protein of 2050 amino acids, and truncated variants thereof, such as a vWF fragment sufficient to stabilize endogenous FVIII levels in vWF-deficient mice, e.g, a truncated variant containing the D′D3 region (amino acids 764-1247) or the D1D2D′D3 region; and vWF variants with one or more amino acid substitutions, e.g., in the D′ region as described in U.S. Pat. No. 9,458,223. A variant vWF protein preferably has at least 50%, 75%, 90% or more (including >100%) of a biological activity of the corresponding wild-type vWF protein. Art-recognized assays for determining the biological activity of a vWF include ristocetin co-factor activity (Federici A B et al. 2004. Haematologica 89:77-85), binding of vWF to GP Ibα of the platelet glycoprotein complex Ib-V-IX (Sucker et al. 2006. Clin Appl Thromb Hemost. 12:305-310), and collagen binding (Kallas & Talpsep. 2001. Annals of Hematology 80:466-471). In some embodiments, the vWF protein produced by an engineered MSFC of the disclosure comprises a naturally-occurring or variant vWF amino acid sequence fused to a heterologous polypeptide or non-polypeptide moiety extending the half-life of the vWF protein. Exemplary half-life extending moieties include Fc, albumin, a PAS sequence, transferrin, CTP (28 amino acid C-terminal peptide (CTP) of human chorionic gonadotropin (hCG) with its 4 O-glycans), polyethylene glycol (PEG), hydroxyethyl starch (HES), albumin binding polypeptide, albumin-binding small molecules, or any combination thereof.

Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”), 1 to 10 carbon atoms (“C₁-C₁₂ alkyl”), 1 to 8 carbon atoms (“C₁-C₈ alkyl”), 1 to 6 carbon atoms (“C₁-C₆ alkyl”), 1 to 5 carbon atoms (“C₁-C₅ alkyl”), 1 to 4 carbon atoms (“C₁-C₄alkyl”), 1 to 3 carbon atoms (“C₁-C₃ alkyl”), 1 to 2 carbon atoms (“C₁-C₂ alkyl”), or 1 carbon atom (“C₁ alkyl”). In some embodiments, an alkyl group has 2 to 6 carbon atoms (“C₂-C₆alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”), 2 to 10 carbon atoms (“C₂-C₁₀ alkenyl”), 2 to 8 carbon atoms (“C₂-C₈ alkenyl”), 2 to 6 carbon atoms (“C₂-C₆ alkenyl”), 2 to 5 carbon atoms (“C₂-C₅ alkenyl”), 2 to 4 carbon atoms (“C₂-C₄ alkenyl”), 2 to 3 carbon atoms (“C₂-C₃ alkenyl”), or 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”), 2 to 10 carbon atoms (“C₂-C₁₀ alkynyl”), 2 to 8 carbon atoms (“C₂-C₈ alkynyl”), 2 to 6 carbon atoms (“C₂-C₆ alkynyl”), 2 to 5 carbon atoms (“C₂-C₅ alkynyl”), 2 to 4 carbon atoms (“C₂-C₄ alkynyl”), 2 to 3 carbon atoms (“C₂-C₃ alkynyl”), or 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —N(R^{C})(R^{D}), or the like, it will be understood that the terms heteroalkyl and —CH₂O or —N(R^{C})(R^{D}) are not redundant or mutually exclusive. Rather, the specific heteroalkyl groups are recited to add clarity. Thus, the term “heteroalkyl” should not be interpreted herein as excluding specific heteroalkyl groups, such as —CH₂O, —N(R^{C})(R^{D}), or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

The terms “alkylene,” “alkenylene,” “alkynylene,” or “heteroalkylene,” alone or as part of another substituent, mean, unless otherwise stated, a divalent radical derived from an alkyl, alkenyl, alkynyl, or heteroalkyl, respectively. An alkylene, alkenylene, alkynylene, or heteroalkylene group may be described as, e.g., a C₁-C₆-membered alkylene, C₂-C₆-membered alkenylene, C₂-C₆-membered alkynylene, or C₁-C₆-membered heteroalkylene, wherein the term “membered” refers to the non-hydrogen atoms within the moiety. In the case of heteroalkylene groups, heteroatoms can also occupy either or both chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′— and —R′C(O)₂—.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, the terms “arylene” and “heteroarylene,” alone or as part of another substituent, mean a divalent radical derived from an aryl and heteroaryl, respectively. As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈cycloalkyl”), 3 to 6 ring carbon atoms (“C₃-C₆ cycloalkyl”), or 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, piperazinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl or thiomorpholinyl-1,1-dioxide. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Amino” as used herein refers to the radical —NR⁷⁰R⁷¹, wherein R⁷⁰ and R⁷¹ are each independently hydrogen, C₁-C₁₂ alkyl, C₃-C₁₂ cycloalkyl, C₄-C₁₂ heterocyclyl, C₆-C₁₂ aryl, and C₅-C₁₂ heteroaryl. In some embodiments, amino refers to NH₂.

As used herein, “cyano” refers to the radical —CN.

As used herein, “halo” or “halogen,” independently or as part of another substituent, mean, unless otherwise stated, a fluorine (F), chlorine (C₁), bromine (Br), or iodine (I) atom.

As used herein, “hydroxy” refers to the radical —OH.

Alkyl, alkenyl, alkynyl, heteroalkyl, cycloalkyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Compounds of Formula (I) described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

Compounds of Formula (I) described herein may also comprise one or more isotopic substitutions. For example, H may be in any isotopic form, including ¹H, ²H (D or deuterium), and ³H (T or tritium); C may be in any isotopic form, including ¹²C, ¹³C, ¹⁴C; O may be in any isotopic form, including ¹⁶O and ¹⁸O; and the like.

The term “pharmaceutically acceptable salt” is meant to include salts of the active compounds that are prepared with relatively nontoxic acids or bases, depending on the particular substituents found on the compounds described herein. When compounds used in present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds used in present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, e.g., Berge et al, Journal of Pharmaceutical Science 66: 1-19 (1977)). Certain specific compounds used in the present disclosure contain both basic and acidic functionalities that allow the compounds to be converted into either base or acid addition salts. These salts may be prepared by methods known to those skilled in the art. Other pharmaceutically acceptable carriers known to those of skill in the art are suitable for use in the present disclosure.

In addition to salt forms, the present disclosure may employ compounds of Formula (I) in a prodrug form. Prodrugs are those compounds that readily undergo chemical changes under physiological conditions to provide the compounds of the present disclosure. Additionally, prodrugs can be converted to useful compounds of Formula (I) by chemical or biochemical methods in an ex vivo environment.

Certain compounds of Formula (I) described herein can exist in unsolvated forms as well as solvated forms, including hydrated forms. In general, the solvated forms are equivalent to unsolvated forms and are encompassed within the scope of the present disclosure. Certain compounds of Formula (I) described herein may exist in multiple crystalline or amorphous forms. In general, all physical forms are equivalent for the uses contemplated by the present disclosure and are intended to be within the scope of the present disclosure.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound and wherein x is a number greater than 0.

The term “tautomer” as used herein refers to compounds that are interchangeable forms of a compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest. The symbol “ [symbol] ” as used herein refers to a connection to an entity, e.g., a polymer (e.g., hydrogel-forming polymer such as alginate) or an implantable element (e.g., a device or material). The connection represented by “ [symbol] ” may refer to direct attachment to the entity, e.g., a polymer or an implantable element, may refer to linkage to the entity through an attachment group. An “attachment group,” as described herein, refers to a moiety for linkage of a compound of Formula (I) to an entity (e.g., a polymer or an implantable element as described herein), and may comprise any attachment chemistry known in the art. A listing of exemplary attachment groups is outlined in Bioconjugate Techniques (3^{rd} ed, Greg T. Hermanson, Waltham, Mass.: Elsevier, Inc, 2013), which is incorporated herein by reference in its entirety. In some embodiments, an attachment group comprises alkyl, alkenyl, alkynyl, heteroalkyl, cycloalkyl, heterocyclyl, aryl, heteroaryl, —C(O)—, —OC(O)—, —N(R^{C})—, —N(R^{C})C(O)—, —C(O)N(R^{C})—, —N(R^{C})N(R^{D})—, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, wherein each of R^{A}, R^{C}, R^{D}, R^{F}, R^{G}, x and y is independently as described herein. In some embodiments, an attachment group comprises an amine, ketone, ester, amide, alkyl, alkenyl, alkynyl, or thiol. In some embodiments, an attachment group is a cross-linker. In some embodiments, the attachment group is —C(O)(C₁-C₆-alkylene)-, wherein alkylene is substituted with R¹, and R¹ is as described herein. In some embodiments, the attachment group is —C(O)(C₁-C₆-alkylene)-, wherein alkylene is substituted with 1-2 alkyl groups (e.g., 1-2 methyl groups). In some embodiments, the attachment group is —C(O)C(CH₃)₂—. In some embodiments, the attachment group is —C(O)(methylene)-, wherein alkylene is substituted with 1-2 alkyl groups (e.g., 1-2 methyl groups). In some embodiments, the attachment group is —C(O)CH(CH₃)—. In some embodiments, the attachment group is —C(O)C(CH₃)—.

### Mesenchymal Stem Function Cells

Disclosed herein are cell compositions comprising mesenchymal stem function cells (MSFCs), e.g., mesenchymal stem cells (MSCs) or cells derived from MSCs, including engineered MSCs or engineered cells derived from MSCs, compositions thereof, implantable elements comprising the same, and methods of making or manufacturing and using such cells, compositions and implantable elements. In an embodiment, an MSFC, e.g., an MSC, is an engineered MSFC, e.g., an engineered MSC.

In an embodiment, an MSFC (e.g., an MSC) is found in or derived from the bone marrow, blood, dental pulp cells, adipose tissue, skin, spleen, pancreas, brain, kidney, liver, heart, retina, brain, hair follicles, intestine, lung, lymph node, thymus, bone, ligament, tendon, skeletal muscle, dermis, and periosteum. Exemplary MSFCs include, in an embodiment, a mesenchymal stem cell, a mesenchymal stromal cell, or a pericyte. In an embodiment, exemplary MSFCs (e.g., MSCs) include an adipose-derived MSFC (e.g., an adipose-derived MSC); a placental-derived MSFC (e.g., a placental-derived MSC); an amniotic-derived MSFC (e.g., a amniotic-derived MSC); a blood-derived MSFC (e.g., a blood-derived MSC); a bone marrow-derived MSFC (a bone marrow-derived MSC); a hematopoietic stem cell-derived MSFC (e.g., a hematopoietic stem cell-derived MSC); a muscle-derived MSFC (e.g., a muscle-derived MSC); or a cartilage-derived MSFC (e.g., a cartilage-derived MSC).

MSFCs can be derived from stem cells, less differentiated cells, de-differentiated cells, or from trans-differentiation from a differentiated cell.

An MSFC, e.g., an MSC, can be derived from primary cells, dedifferentiated cells or from differentiated tissue that was dedifferentiated into a less differentiated mesodermal lineage.

In an embodiment, the MSFC, e.g., an MSC, is derived from an iPS cell. In an embodiment, the MSFC, e.g., an MSC, is derived from an embryonic stem cell.

In an embodiment, the MSFC is a non-primary MSC, having an antigen profile comprising less than about 1.25% CD45+ cells (or less than about 0.75% CD45+), at least about 95% CD105+ cells, and at least about 95% CD166+ cells.

In an embodiment, the MSFC is multipotent. Multipotency can be assessed by any assay known to one of skill in the art. In an embodiment, the MSFC is not totipotent.

An MSFC can be an engineered cell, such as a cell engineered to express a protein or nucleic acid, or a cell engineered to produce a metabolic product. An MSFC can be a mammalian cell, e.g., a human cell. An engineered MSFC can be a mammalian cell, e.g., a human cell. In an embodiment, the MSFC, e.g., an MSC, described herein is not engineered.

In an embodiment, an engineered MSFC is an MSFC (or is derived from an MSFC) that comprises at least one exogenous transcription unit, which may be present in an extra-chromosomal expression vector, or integrated into one or more chromosomal sites in the cell. In an embodiment, the transcription unit comprises a promoter operably linked to a coding sequence for a polypeptide.

In an embodiment, an MSFC is derived from a culture in which at least 10, 20, 30, 40, 50, 60, 79, 80, 90, 95, 98, or 99% of the cells in the culture are MSFCs or engineered MSFCs. In an embodiment, an MSFC is derived from a culture in which at least 10, 20, 30, 40, 50, 60, 79, 80, 90, 95, 98, or 99% of the cells in the culture are MSFCs or non-engineered MSFCs. In an embodiment, a culture comprises MSFCs, or engineered MSFCs, and a second cell type, e.g., a feeder cell or a contaminating cell. In an embodiment, an MSFC is an MSC, e.g., an engineered or non-engineered MSC derived from an individual, e.g., the same or a different individual to whom the cells are administered.

In an embodiment, the MSFC comprises a commercially available MSC. An MSFC can be derived from any of a variety of commercially available MSCs. In some embodiments, the MSFC comprises an MSC deposited with or available from a cell bank or other cell depository, e.g., the American Type Culture Collection (ATCC) or the European Collection of Cell Cultures (ECACC). Exemplary human MSCs include the bone marrow- and adipose-derived human MSCs marketed by RoosterBio (Frederick, Md.), human MSCs marketed by ATCC (Manassas, Va.) that are derived from adipose, bone marrow or umbilical, human MSC lines marketed by MilliporeSigma (Burlington, Mass.) that are derived from bone marrow, embryonic or adipose tissue, human MSCs marketed by Axol Bioscience (Cambridge, United Kingdom) that are derived from adipose, bone marrow, umbilical cord or pre-adipocytes, MSCs derived from adult human bone marrow that are marketed by Hemacare (Van Nuys, Calif.), and Poietics™ Normal human bone marrow derived MSCs marketed by Lonza (Basel, Switzerland).

In an embodiment, an MSFC expresses a biomarker, e.g., an antigen that is characteristic of an MSC cell, e.g., a naturally occurring MSC cell. In some embodiments, the biomarker (e.g., antigen) is a protein. Exemplary biomarkers include CD105, CD106, CD73, CD90, Stro-1, CD49a, CD29, CD44, CD146, CD166, TNAP+, THY-1+, Stro-2, Stro-4, and alkaline phosphatase. In an embodiment, an MSFC does not express significant levels of it does not express significant levels of one, two, three, or any of CD34, CD31, VE-cadherin, CD45, HLA-DR, CD11b and a glycophorin or leukocyte differentiation antigen, e,g, CD14, CD33, CD3 or CD19. In an embodiment, the MSFC expresses one, two, or all of CD75, CD90, and CD105 and does not express one, two, or any of CD45, CD34, and CD14.

In an embodiment, the MSFC is not in a terminal state of differentiation; can terminally differentiate into one or more cell types; and expresses a marker from the group of CD105, CD73, CD90, Stro-1, CD49a, CD29 or CD166, CD75, CD90, CD105 CD45, HLA-DR, CD11b, CD19, CD34, and CD14.

In an embodiment, the MSFC is not in a terminal state of differentiation; can terminally differentiate into one or more cell types; and expresses a marker from the group of CD105, CD73, CD90, Stro-1, CD49a, CD29 and CD166.

In an embodiment, the MSFC is not in a terminal state of differentiation; can terminally differentiate into one or more cell types; and expresses a marker from the group of CD75, CD90, and CD105.

In an embodiment, the MSFC is not in a terminal state of differentiation; can terminally differentiate into one or more cell types; and expresses a marker from the group of CD34, CD45, HLA-DR, CD11b, and CD19.

In an embodiment, the MSFC is not in a terminal state of differentiation; can terminally differentiate into one or more cell types; and expresses a marker from the group of CD45, CD34, and CD14.

In an embodiment, the MSFC is not in a terminal state of differentiation; can terminally differentiate into one or more cell types; and expresses a marker from the group of CD75, CD90, and CD105.

In an embodiment, the MSFC (e.g., MSC) comprises a human bone marrow derived MSC showing fibroblast-like morphology, e.g., as seen under phase contrast microscope (Carl Zeiss Axiovert 40 CFL), e.g., at 63× magnification. In an embodiment, the MSFC (e.g., MSC) is characterized morphologically by a small cell body with a few cell processes that are long and thin. In an embodiment, the MSFC cell body contains a large, round nucleus with a prominent nucleolus, which is surrounded by finely dispersed chromatin particles, giving the nucleus a clear appearance. In an embodiment, the remainder of the MSFC cell body contains a small amount of Golgi apparatus, rough endoplasmic reticulum, mitochondria, and polyribosomes. In an embodiment, the MSFCs, which are long and thin, are widely dispersed and the adjacent extracellular matrix is populated by a few reticular fibrils but is devoid of the other types of collagen fibrils.

In an embodiment, the MSFCs (e.g., MSCs) have a phenotype characterized by one or more of the following properties: a) TNFR1 expression; b) inhibition of IL2R.alpha (e.g. in a PBMC assay); c) greater than 70% viability after freeze-thaw; d) the ability to maintain phenotype following ex vivo expansion; e) the capacity for differentiation; and f) are isogenic (i.e., derived from a single donor).

In an embodiment, the MSFC (e.g., the MSC) is formulated into a composition (e.g., an MSFC composition). In an embodiment, the MSFC composition comprises at least about 1 billion (1×10⁹) cells. In one embodiment, the MSFC composition has an antigen profile comprising less than about 0.75% CD45+ cells, at least about 95% CD105+ cells, or at least about 95% CD166+ cells.

In an embodiment, a plurality of MSFCs, e.g., engineered MSFCs, have or are provided in a preselected form factor or a form factor described herein. In an embodiment, the form factor is a monolayer or cluster. A “cluster of MSFCs, e.g., a cluster of MSCs,” as used herein, refers to a plurality of MSFCs or an aggregate of MSFCs typically having a ratio of cells to surface area of the form factor that is lower than that of a monolayer. In some embodiments, a cluster of MSFCs comprises at least about 2, 3, 4, 5, 10, 50, 100, 200, 300, 400, 500, 1,000, 2,000, 3,000, 4,000, or 5,000 MSFCs. In some embodiments, the cluster of MSFCs comprises between 2 and 5,000 cells, 2 and 1,000 cells, 5 and 1,000 cells, 5 and 500 cells, 10 and 500 cells. In some embodiments, the cluster of MSFCs comprises between 2 and 10 cells, 5 and 10 cells, about 5 and 20 cells, 5 and 50 cells, or 10 and 100 cells. In some embodiments, the cluster of MSFCs comprises 50 to 100 cells, 50 to 250 cells, 100 to 500 cells, 100 to 1,000 cells, or 500 to 1,000 cells. In an embodiment, the lower, upper, or both, endpoints of a range of number of cells is an average and can vary by 5%. In an embodiment, the lower, upper, or both, endpoints of a range of number of cells is an average and can vary by 10%.

In an embodiment, a cluster of MSFCs has a spheroid, globular, or ellipsoid shape, or any other shape with a curved surface. In some embodiments, the cluster of MSFCs has a spheroid shape, wherein at least about 10%, 15%, 20%, 25%, 30%, 35%, 40%, 45%, 50%, 55%, 60%, 65%, 70%, 75%, 80%, 85%, 90%, 95% or 100% of the cells in the cluster of MSFCs conform to the spheroid shape. In some embodiments, the cluster of MSFCs has a globular shape, wherein at least about 10%, 15%, 20%, 25%, 30%, 35%, 40%, 45%, 50%, 55%, 60%, 65%, 70%, 75%, 80%, 85%, 90%, 95% or 100% of the cells in the cluster of MSFCs conform to the globular shape. In some embodiments, the cluster of MSFCs has a ellipsoid shape, wherein at least about 10%, 15%, 20%, 25%, 30%, 35%, 40%, 45%, 50%, 55%, 60%, 65%, 70%, 75%, 80%, 85%, 90%, 95% or 100% of the cells in the cluster of MSFCs conform to the ellipsoid shape.

In an embodiment, a cluster of MSFCs comprises certain dimensions, e.g., with a range of sizes in each of the x dimension, y dimension, or z dimension. In some embodiments, the length of at least one of the x, y, or z dimensions is independently greater than about 10 μm (e.g., greater than about 15 μm, about 20 μm, about 30 μm, about 40 μm, about 50 μm, about 75 μm, about 100 μm, about 250 μm, about 500 μm, about 750 μm, about 1 mm, about 1.1 mm, about 1.2 mm, about 1.3 mm, about 1.4 mm, about 1.5 mm, or more). In some embodiments, the length of at least one of the x, y, or z dimensions cluster of MSFCs is independently less than about 2 mm (e.g., less than about 1.5 mm, about 1.4 mm, about 1.3 mm, about 1.2 mm, about 1.1 mm, about 1.0 mm, about 750 μm, about 500 μm, about 250 μm, about 100 μm, about 75 μm, about 50 μm, about 40 μm, about 30 μm, about 20 μm, or less).

In some embodiments, the length of at least one of the x, y, or z dimensions of the cluster of MSFCs is independently between about 10 μm to about 5 mm in size (e.g., between about 20 μm to about 4 mm, about 50 μm to about 2 mm, or about 100 μm to about 1.5 mm). In some embodiments, the length of at least two of the x, y, or z dimensions of the cluster of MSFCs is independently between about 10 μm to about 5 mm in size (e.g., between about 20 μm to about 4 mm, about 50 μm to about 2 mm, or about 100 μm to about 1.5 mm). In some embodiments, the length of all three of the x, y, or z dimensions of the cluster of MSFCs is independently between about 10 μm to about 5 mm in size (e.g., between about 20 μm to about 4 mm, about 50 μm to about 2 mm, or about 100 μm to about 1.5 mm).

In some embodiments, each of the dimensions of the cluster of MSFCs are independently within about 5% (e.g., about 10%, about 15%, about 20%, about 25%, about 30%, about 35%, about 40%, about 45%, about 50% about 60%, about 70%, about 80%, about 90%, or about 95%) of the other dimensions. For example, the x dimension of the cluster of MSFCs may be about 5% (e.g., about 10%, about 15%, about 20%, about 25%, about 30%, about 35%, about 40%, about 45%, about 50% about 60%, about 70%, about 80%, about 90%, or about 95%) of both the y dimension and the z dimension. In some embodiments, the y dimension of the cluster of MSFCs may be about 5% (e.g., about 10%, about 15%, about 20%, about 25%, about 30%, about 35%, about 40%, about 45%, about 50% about 60%, about 70%, about 80%, about 90%, or about 95%) of both the x dimension and the z dimension. In other embodiments, the z dimension of the cluster of MSFCs may be about 5% (e.g., about 10%, about 15%, about 20%, about 25%, about 30%, about 35%, about 40%, about 45%, about 50% about 60%, about 70%, about 80%, about 90%, or about 95%) of both the x dimension and the y dimension.

The cluster of MSFCs may be embedded in a matrix, e.g., an extracellular matrix secreted by an MSFC (e.g., a cluster of embedded MSFCs). In some embodiments, the cluster of MSFCs is encapsulated by a matrix, e.g., an extracellular matrix secreted by an MSFC (e.g., a cluster of encapsulated MSFCs). In some embodiments, the extracellular matrix comprises proteins, e.g., collagen (e.g., a structural collagen or an angiostatic collagen, e.g., collagen IV, collagen III, collagen V, collagen VI, collagen XVIII), laminin, elastin, integrin, or fibronectin. The extracellular matrix or a component thereof may be either naturally occurring or non-naturally occurring. In some embodiments, the extracellular matrix or a component thereof is naturally occurring and is supplemented by a non-naturally occurring component. In other embodiments, the extracellular matrix or a component thereof is non-naturally occurring and is supplemented by a naturally occurring component.

MSFCs for use in compositions and methods described herein, e.g., for use in a plurality of MSFCs having a preselected form factor or a form factor described herein, e.g., a cluster of MSFCs, may be in various stages of the cell cycle. In some embodiments, at least one MSFC in the cluster of MSFCs is undergoing cell division. Cell division may be measured using any known method in the art, e.g., as described in DeFazio A et al (1987) J Histochem Cytochem 35:571-577 and Dolbeare Fetal (1983) Proc Natl Acad Sci USA 80:5573-5577, each of which is incorporated by reference in its entirety. In an embodiment, at least 1, 2, 3, 4, 5, 10, or 20% of the MSFCs are undergoing cell division, e.g., as determined by 5-ethynyl-2′deoxyuridine (EdU) assay or 5-bromo-2′-deoxyuridine (BrdU) assay. In some embodiments, cell proliferation is visualized or quantified by microscopy (e.g., fluorescence microscopy (e.g., time-lapse or evaluation of spindle formation) or flow cytometry. In some embodiments, none of the MSFCs in the cluster of MSFCs are undergoing cell division and are quiescent. In an embodiment, less than 1, 2, 3, 4, 5, 10, or 20% of the MSFCs are undergoing cell division, 5-ethynyl-2′deoxyuridine (EdU) assay, 5-bromo-2′-deoxyuridine (BrdU) assay, microscopy (e.g., fluorescence microscopy (e.g., time-lapse or evaluation of spindle formation), or flow cytometry.

In some embodiments, the MSFCs in the cluster of MSFCs are capable of autophagy. Autophagy may be measured using any known method in the art, e.g., as described in Barth et al (2010) J. Pathol 221:117-124, which is incorporated by reference in its entirety. For example, autophagy may be determined or quantified by a 5-ethynyl-2′deoxyuridine (EdU) assay, a 5-bromo-2′-deoxyuridine (BrdU) assay, a cationic amphiphilic tracer (CAT) assay, in which the dye rapidly partitions into cells and selectively labels vacuoles associated with the autophagy pathway. In some embodiments, autophagy is visualized or quantified by microscopy (e.g., fluorescence microscopy (e.g., time-lapse or evaluation of spindle formation)). In an embodiment, at least 1, 2, 3, 4, 5, 10, or 20% of the MSFCs are capable of autophagy, e.g., as determined by 5-ethynyl-2′deoxyuridine (EdU) assay, 5-bromo-2′-deoxyuridine (BrdU) assay, cationic amphiphilic tracer (CAT) assay, or microscopy (e.g., fluorescence microscopy (e.g., time-lapse or evaluation of spindle formation).

In some embodiments, the MSCs in the cluster of MSFCs are capable of phagocytosis. Phagocytosis may be measured using any known method in the art, e.g., as described in Oda T and Maeda H (1986) J Immunol Methods 88:175-183 and Nuutila J and Lilius E M (2005) Cytometry A (2005) 65:93-102, each of which is incorporated by reference in its entirety. For example, phagocytosis may be measured by a 5-ethynyl-2′deoxyuridine (EdU) assay, a 5-bromo-2′-deoxyuridine (BrdU) assay, or a fluorescein-labeled antibody assay, in which the uptake of a labeled substance via the phagocytotic pathway is monitored. In some embodiments, phagocytosis is visualized or quantified by microscopy (e.g., fluorescence microscopy (e.g., time-lapse or evaluation of spindle formation) or flow cytometry. In an embodiment, at least 1, 2, 3, 4, 5, 10, or 20% of the MSFCs are capable of phagocytosis, e.g., as determined by a5-ethynyl-2′deoxyuridine (EdU) assay, a 5-bromo-2′-deoxyuridine (BrdU) assay, a fluorescein-labeled antibody assay, microscopy (e.g., fluorescence microscopy (e.g., time-lapse or evaluation of spindle formation), or flow cytometry.

In an embodiment, at least 1, 2, 3, 4, 5, 10, or 20% of the MSFCs are viable. Cell viability may be measured using any known method in the art, e.g., as described in Riss, T. et al (2013) “Cell Viability Assays” in Assay Guidance Manual (Sittapalam, G. S. et al, eds). For example, cell viability may be measured or quantified by an ATP assay, 5-ethynyl-2′deoxyuridine (EdU) assay, 5-bromo-2′-deoxyuridine (BrdU) assay. In some embodiments, cell viability is visualized or quantified by microscopy (e.g., fluorescence microscopy (e.g., time-lapse or evaluation of spindle formation) or flow cytometry. In an embodiment, at least 1, 2, 3, 4, 5, 10, or 20% of the MSFCs are viable, e.g., as determined by an ATP assay, a 5-ethynyl-2′deoxyuridine (EdU) assay, a 5-bromo-2′-deoxyuridine (BrdU) assay, microscopy (e.g., fluorescence microscopy (e.g., time-lapse or evaluation of spindle formation), or flow cytometry.

Any of the parameters described herein may be assessed using standard techniques known to one of skill in the art, such as histology, microscopy, and various functional assays.

In some embodiments, the MSFCs having a form factor, e.g., in a cluster of MSFCs, exhibit polarity. For example, the MSFCs having a form factor may exhibit the polarity characteristics in situ in the eye (e.g., the retina). In an embodiment, at least 1, 2, 3, 4, 5, 10, or 20% of the MSFCs exhibit polarity, e.g., as determined by art known methods, e.g., art known staining and microscopy assays. In some embodiments, the MSFCs having a form factor, e.g., in a cluster of MSFCs, do not exhibit polarity. In an embodiment, at least 1, 2, 3, 4, 5, 10, or 20% of the MSFCs exhibit polarity, e.g., as determined by art known methods, e.g., art known staining and microscopy assays.

An MSFC (e.g., an MSC), or an engineered MSFC (e.g., an engineered MSC) may be disposed on a non-cellular carrier (e.g, a microcarrier). In some embodiments, the microcarrier is a bead. In some embodiments, the microcarrier comprises a polymer, e.g., plastic (e.g., polystyrene, polyethylene, polyester, polypropylene), glass, acrylamide, silica, silicone rubber, cellulose, dextran, collagen (e.g., gelatin), or a glycosaminoglycan. The microcarrier may be any shape or configuration, including a sphere (e.g., a bead), flat disc, fiber, woven disc, or cube. In some embodiments, the microcarrier may have a polar surface or a charged surface (e.g., a negative charge or a positive charge). In some embodiments, the microcarrier may have a smooth surface or a textured surface. In some embodiments, an MSFC (e.g., an engineered MSFC) is attached to a microcarrier through adsorption of the cell surface proteins (e.g., glycoproteins, e.g., fibronectin) to the microcarrier surface. The microcarrier may range in size from about 10 μm to about 5 mm (e.g., between about 10 μm to about 3 mm, 10 μm to about 1 mm, 50 μm to about 1 mm, 100 μm to about 1 mm, 100 μm to about 500 μm).

An MSFC may be disposed on a microcarrier (e.g., a bead, e.g., a polystyrene bead, e.g., a Cytodex 1 microcarrier) using any known method in the art (see, e.g., Nilsson, K. (1988) Biotechnol Engineering Rev 6:404-439. For example, a small amount (e.g., about 1 g, about 5 g) of microcarrier may be weighed out, washed with a buffer, and sterilized (e.g., via autoclave). The sterile microcarrier may then be washed several times with buffer and media prior to introducing a population of MSFCs (e.g., about 10 million MSFCs, about 25 million MSFCs, about 40 million MSFCs, about 100 million MSFCs). The mixture of microcarrier and cells can then be gently mixed and incubated (e.g., in a stationary incubator) at a specified temperature (e.g., at 25° C., at 37° C.). After incubation, the cells and microcarrier mixture may be transferred flask and gently stirred until incorporation into or within an implantable element (e.g., an implantable element described herein).

### Therapeutic Agents

The present disclosure features an MSFC that produces or is capable of producing a therapeutic agent for the prevention or treatment of a disease, disorder, or condition described herein. In an embodiment, the MSFC is an engineered MSFC (e.g., an engineered MSC). The therapeutic agent may be any biological substance, such as a nucleic acid (e.g., a nucleotide, DNA, or RNA), a polypeptide, a lipid, a sugar (e.g., a monosaccharide, disaccharide, oligosaccharide, or polysaccharide), or a small molecule, each of which are further elaborated below. Exemplary therapeutic agents include the agents listed in WO 2017/075631.

In some embodiments, the MSFCs (e.g., engineered MSFCs) produce a nucleic acid. A nucleic acid produced by an MSFC described herein may vary in size and contain one or more nucleosides or nucleotides, e.g., greater than 2, 3, 4, 5, 10, 25, 50, or more nucleosides or nucleotides. In some embodiments, the nucleic acid is a short fragment of RNA or DNA, e.g., and may be used as a reporter or for diagnostic purposes. Exemplary nucleic acids include a single nucleoside or nucleotide (e.g., adenosine, thymidine, cytidine, guanosine, uridine monophosphate, inosine monophosphate), RNA (e.g., mRNA, siRNA, miRNA, RNAi), and DNA (e.g., a vector, chromosomal DNA). In some embodiments, the nucleic acid has an average molecular weight of about 0.25 kD, 0.5 kD, 1 kD, 1.5 kD, 2 kD, 2.5 kD, 5 kD, 10 kD, 25 kD, 50 kD, 100 kD, 150 kD, 200 kD, or more.

In some embodiments, the therapeutic agent is a peptide or polypeptide (e.g., a protein), such as a hormone, enzyme, cytokine (e.g., a pro-inflammatory cytokine or an anti-inflammatory cytokine), growth factor, clotting factor, or lipoprotein. A peptide or polypeptide (e.g., a protein, e.g., a hormone, growth factor, clotting factor or coagulation factor, antibody molecule, enzyme, cytokine, cytokine receptor, or a chimeric protein including cytokines or a cytokine receptor) produced by an MSFC can have a naturally occurring amino acid sequence, or may contain a variant of the naturally occurring sequence. The variant can be a naturally occurring or non-naturally occurring amino acid substitution, mutation, deletion or addition relative to the reference naturally occurring sequence. The naturally occurring amino acid sequence may be a polymorphic variant. The naturally occurring amino acid sequence can be a human or a non-human amino acid sequence. In some embodiments, the naturally occurring amino acid sequence or naturally occurring variant thereof is a human sequence. In addition, a peptide or polypeptide (e.g., a protein) for use with the present disclosure may be modified in some way, e.g., via chemical or enzymatic modification (e.g., glycosylation, phosphorylation). In some embodiments, the peptide has about 2, 3, 4, 5, 6, 7, 8, 9, 10, 12, 14, 16, 18, 20, 25, 30, 35, 40, 45, or 50 amino acids. In some embodiments, the protein has an average molecular weight of 5 kD, 10 kD, 25 kD, 50 kD, 100 kD, 150 kD, 200 kD, 250 kD, 500 kD, or more.

In some embodiments, the protein is a hormone. Exemplary hormones include anti-diuretic hormone (ADH), oxytocin, growth hormone (GH), prolactin, growth hormone-releasing hormone (GHRH), thyroid stimulating hormone (TSH), thyrotropin-release hormone (TRH), adrenocorticotropic hormone (ACTH), follicle-stimulating hormone (FSH), luteinizing hormone (LH), luteinizing hormone-releasing hormone (LHRH), thyroxine, calcitonin, parathyroid hormone, aldosterone, cortisol, epinephrine, glucagon, insulin, estrogen, progesterone, and testosterone. In some embodiments, the protein is insulin (e.g., insulin A-chain, insulin B-chain, or proinsulin). In some embodiments, the protein is a growth hormone, such as human growth hormone (hGH), recombinant human growth hormone (rhGH), bovine growth hormone, methione-human growth hormone, des-phenylalanine human growth hormone, and porcine growth hormone. In some embodiments, the protein is not insulin (e.g., insulin A-chain, insulin B-chain, or proinsulin).

In some embodiments, the protein is a growth factor, e.g., vascular endothelial growth factor (VEGF), nerve growth factor (NGF), platelet-derived growth factor (PDGF), fibroblast growth factor (FGF), epidermal growth factor (EGF), transforming growth factor (TGF), and insulin-like growth factor-I and -II (IGF-I and IGF-II).

In some embodiments, the protein is a clotting factor or a coagulation factor, e.g., a blood clotting factor or a blood coagulation factor. In some embodiments, the protein is a protein involved in coagulation, i.e., the process by which blood is converted from a liquid to solid or gel. Exemplary clotting factors and coagulation factors include Factor I (e.g., fibrinogen), Factor II (e.g., prothrombin), Factor III (e.g., tissue factor), Factor V (e.g., proaccelerin, labile factor), Factor VI, Factor VII (e.g., stable factor, proconvertin), Factor VIII (e.g., antihemophilic factor A), Factor VIIIC, Factor IX (e.g., antihemophilic factor B), Factor X (e.g., Stuart-Prower factor), Factor XI (e.g., plasma thromboplastin antecedent), Factor XII (e.g., Hagerman factor), Factor XIII (e.g., fibrin-stabilizing factor), von Willebrand factor, prekallikrein, heparin cofactor II, high molecular weight kininogen (e.g., Fitzgerald factor), antithrombin III, and fibronectin. In some embodiments, the protein is an anti-clotting factor, such as Protein C.

In some embodiments, the protein is an antibody molecule. As used herein, the term “antibody molecule” refers to a protein, e.g., an immunoglobulin chain or fragment thereof, comprising at least one immunoglobulin variable domain sequence. The term “antibody molecule” includes, for example, a monoclonal antibody (including a full length antibody which has an immunoglobulin Fc region). In an embodiment, an antibody molecule comprises a full length antibody, or a full length immunoglobulin chain. In an embodiment, an antibody molecule comprises an antigen binding or functional fragment of a full length antibody, or a full length immunoglobulin chain. In an embodiment, an antibody molecule is a monospecific antibody molecule and binds a single epitope, e.g., a monospecific antibody molecule having a plurality of immunoglobulin variable domain sequences, each of which binds the same epitope. In an embodiment, an antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule.

Various types of antibody molecules may be produced by the MSFCs described herein, including whole immunoglobulins of any class, fragments thereof, and synthetic proteins containing at least the antigen binding variable domain of an antibody. The antibody molecule can be an antibody, e.g., an IgG antibody, such as IgG₁, IgG₂, IgG₃, or IgG₄. An antibody molecule can be in the form of an antigen binding fragment including a Fab fragment, F(ab′)2 fragment, a single chain variable region, and the like. Antibodies can be polyclonal or monoclonal (mAb). Monoclonal antibodies may include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity. In some embodiments, the antibody molecule is a single-domain antibody (e.g., a nanobody). The described antibodies can also be modified by recombinant means, for example by deletions, additions or substitutions of amino acids, to increase efficacy of the antibody in mediating the desired function. Exemplary antibodies include anti-beta-galactosidase, anti-collagen, anti-CD14, anti-CD20, anti-CD40, anti-HER2, anti-IL-1, anti-IL-4, anti-IL6, anti-IL-13, anti-IL17, anti-IL18, anti-IL-23, anti-IL-28, anti-IL-29, anti-IL-33, anti-EGFR, anti-VEGF, anti-CDF, anti-flagellin, anti-IFN-α, anti-IFN-β, anti-IFN-γ, anti-mannose receptor, anti-VEGF, anti-TLR1, anti-TLR2, anti-TLR3, anti-TLR4, anti-TLR5, anti-TLR6, anti-TLR9, anti-PDF, anti-PD1, anti-PDL-1, or anti-nerve growth factor antibody. In some embodiments, the antibody is an anti-nerve growth factor antibody (e.g., fulranumab, fasinumab, tanezumab).

In some embodiments, the protein is a cytokine or a cytokine receptor, or a chimeric protein including cytokines or their receptors, including, for example tumor necrosis factor alpha and beta, their receptors and their derivatives, renin; lipoproteins; colchicine; corticotrophin; vasopressin; somatostatin; lypressin; pancreozymin; leuprolide; alpha-1-antitrypsin; atrial natriuretic factor; lung surfactant; a plasminogen activator other than a tissue-type plasminogen activator (t-PA), for example a urokinase; bombesin; thrombin; enkephalinase; RANTES (regulated on activation normally T-cell expressed and secreted); human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; chorionic gonadotropin; a microbial protein, such as beta-lactamase; DNase; inhibin; activin; receptors for hormones or growth factors; integrin; protein A or D; rheumatoid factors; platelet-derived growth factor (PDGF); epidermal growth factor (EGF); transforming growth factor (TGF) such as TGF-α and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; an interferon such as interferon-alpha (e.g., interferon.alpha.2A), -beta, -gamma, -lambda and consensus interferon; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; transport proteins; homing receptors; addressins; fertility inhibitors such as the prostaglandins; fertility promoters; regulatory proteins; antibodies (including fragments thereof) and chimeric proteins, such as immunoadhesins; precursors, derivatives, prodrugs and analogues of these compounds, and pharmaceutically acceptable salts of these compounds, or their precursors, derivatives, prodrugs and analogues. Suitable proteins or peptides may be native or recombinant and include, e.g., fusion proteins.

Examples of a polypeptide (e.g., a protein) produced by an MSFC described herein also include CCL1, CCL2 (MCP-1), CCL3 (MIP-1α), CCL4 (MIP-1β), CCL5 (RANTES), CCL6, CCL7, CCL8, CCL9 (CCL10), CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1 (KC), CXCL2 (SDF1a), CXCL3, CXCL4, CXCL5, CXCL6, CXCL7, CXCL8 (IL8), CXCL9, CXCL10, CXCL11, CXCL12, CXCL13, CXCL14, CXCL15, CXCL16, CXCL17, CX3CL1, XCL1, XCL2, TNFA, TNFB (LTA), TNFC (LTB), TNFSF4, TNFSF5 (CD40LG), TNFSF6, TNFSF7, TNFSF8, TNFSF9, TNFSF10, TNFSF11, TNFSF13B, EDA, IL2, IL15, IL4, IL13, IL7, IL9, IL21, IL3, IL5, IL6, IL11, IL27, IL30, IL31, OSM, LIF, CNTF, CTF1, IL12a, IL12b, IL23, IL27, IL35, IL14, IL16, IL32, IL34, IL10, IL22, IL19, IL20, IL24, IL26, IL29, IFNL1, IFNL2, IFNL3, IL28, IFNA1, IFNA2, IFNA4, IFNA5, IFNA6, IFNA7, IFNA8, IFNA10, IFNA13, IFNA14, IFNA16, IFNA17, IFNA21, IFNB1, IFNK, IFNW1, IFNG, IL1A (IL1F1), IL1B (IL1F2), IL1Ra (IL1F3), IL1F5 (IL36RN), IL1F6 (IL36A), IL1F7 (IL37), IL1F8 (IL36B), IL1F9 (IL36G), IL1F10 (IL38), IL33 (IL1F11), IL18 (IL1G), IL17, KITLG, IL25 (IL17E), CSF1 (M-CSF), CSF2 (GM-CSF), CSF3 (G-CSF), SPP1, TGFB1, TGFB2, TGFB3, CCL3L1, CCL3L2, CCL3L3, CCL4L1, CCL4L2, IL17B, IL17C, IL17D, IL17F, AIMP1 (SCYE1), MIF, Areg, BC096441, Bmp1, Bmp10, Bmp15, Bmp2, Bmp3, Bmp4, Bmp5, Bmp6, Bmp7, Bmp8a, Bmp8b, C1qtnf4, Cc121a, Cc127a, Cd70, Cer1, Cklf, Clcf1, Cmtm2a, Cmtm2b, Cmtm3, Cmtm4, Cmtm5, Cmtm6, Cmtm7, Cmtm8, Crlf1, Ctf2, Ebi3, Edn1, Fam3b, Fas1, Fgf2, Flt31, Gdf10, Gdf11, Gdf15, Gdf2, Gdf3, Gdf5, Gdf6, Gdf7, Gdf9, Gm12597, Gm13271, Gm13275, Gm13276, Gm13280, Gm13283, Gm2564, Gpi1, Grem1, Grem2, Grn, Hmgb1, Ifna11, Ifna12, Ifna9, Ifnab, Ifne, Il17a, Il23a, Il25, Il31, Iltifb, Inhba, Lefty1, Lefty2, Mstn, Nampt, Ndp, Nodal, Pf4, Pglyrp1, Prl7d1, Scg2, Scgb3a1, Slurp1, Spp1, Thpo, Tnfsf10, Tnfsf11, Tnfsf12, Tnfsf13, Tnfsf13b, Tnfsf14, Tnfsf15, Tnfsf18, Tnfsf4, Tnfsf8, Tnfsf9, Tslp, Vegfa, Wnt1, Wnt2, Wnt5a, Wnt7a, Xcl1, epinephrine, melatonin, triiodothyronine, a prostaglandin, a leukotriene, prostacyclin, thromboxane, islet amyloid polypeptide, müllerian inhibiting factor or hormone, adiponectin, corticotropin, angiotensin, vasopressin, arginine vasopressin, atriopeptin, brain natriuretic peptide, calcitonin, cholecystokinin, cortistatin, enkephalin, endothelin, erythropoietin, follicle-stimulating hormone, galanin, gastric inhibitory polypeptide, gastrin, ghrelin, glucagon, glucagon-like peptide-1, gonadotropin-releasing hormone, hepcidin, human chorionic gonadotropin, human placental lactogen, inhibin, somatomedin, leptin, lipotropin, melanocyte stimulating hormone, motilin, orexin, oxytocin, pancreatic polypeptide, pituitary adenylate cyclase-activating peptide, relaxin, renin, secretin, somatostatin, thrombopoietin, thyrotropin, thyrotropin-releasing hormone, vasoactive intestinal peptide, androgen, alpha-glucosidase (also known as acid maltase), glycogen phosphorylase, glycogen debrancher enzyme, phosphofructokinase, phosphoglycerate kinase, phosphoglycerate mutase, lactate dehydrogenase, carnitine palymityl transferase, carnitine, and myoadenylate deaminase.

In some embodiments, the protein is a replacement therapy or a replacement protein. In some embodiments, the replacement therapy or replacement protein is a clotting factor or a coagulation factor, e.g., vWF (comprises a naturally occurring human factor vWF or a variant thereof), Factor VII (e.g., comprises a naturally occurring human Factor VII amino acid sequence or a variant thereof), Factor VIII (e.g., comprises a naturally occurring human Factor VIII amino acid sequence or a variant thereof) or Factor IX (e.g., comprises a naturally occurring human Factor IX amino acid sequence or a variant thereof).

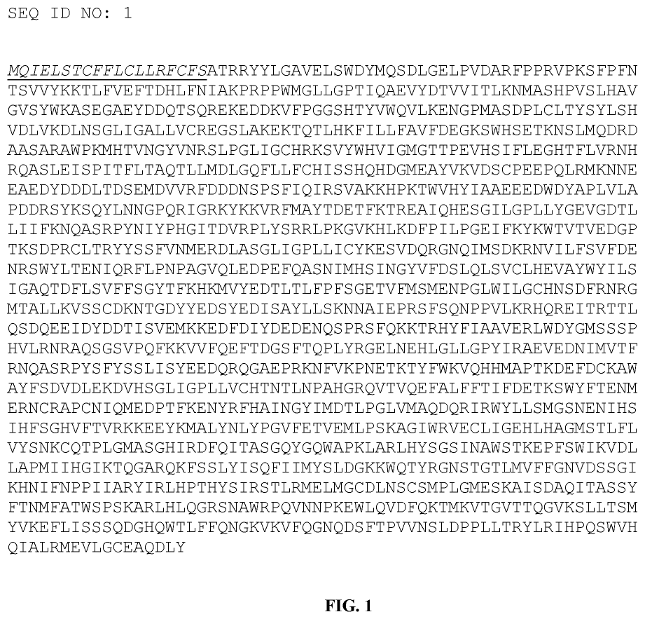

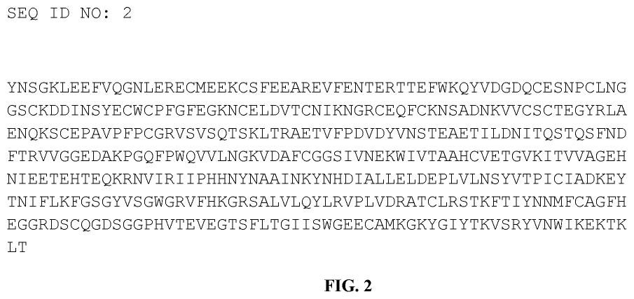

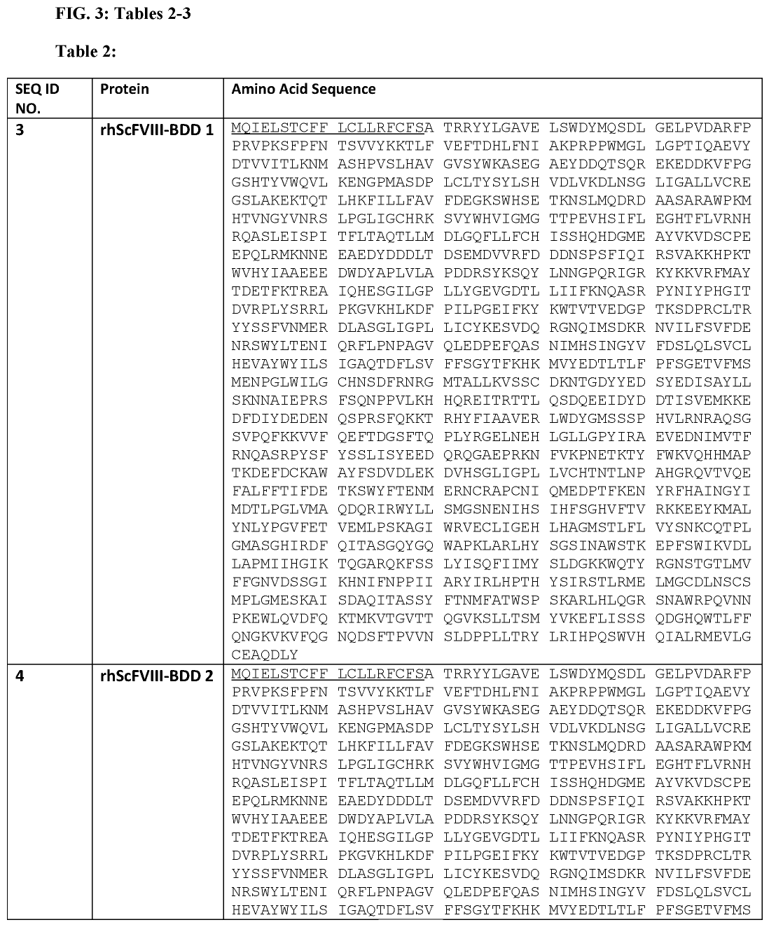

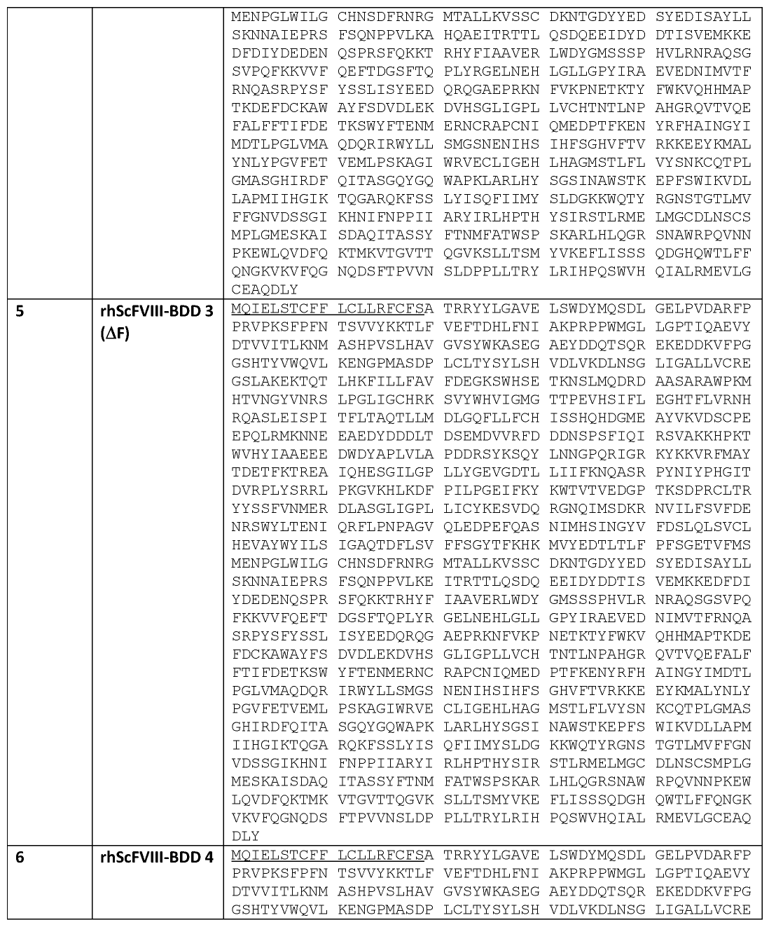

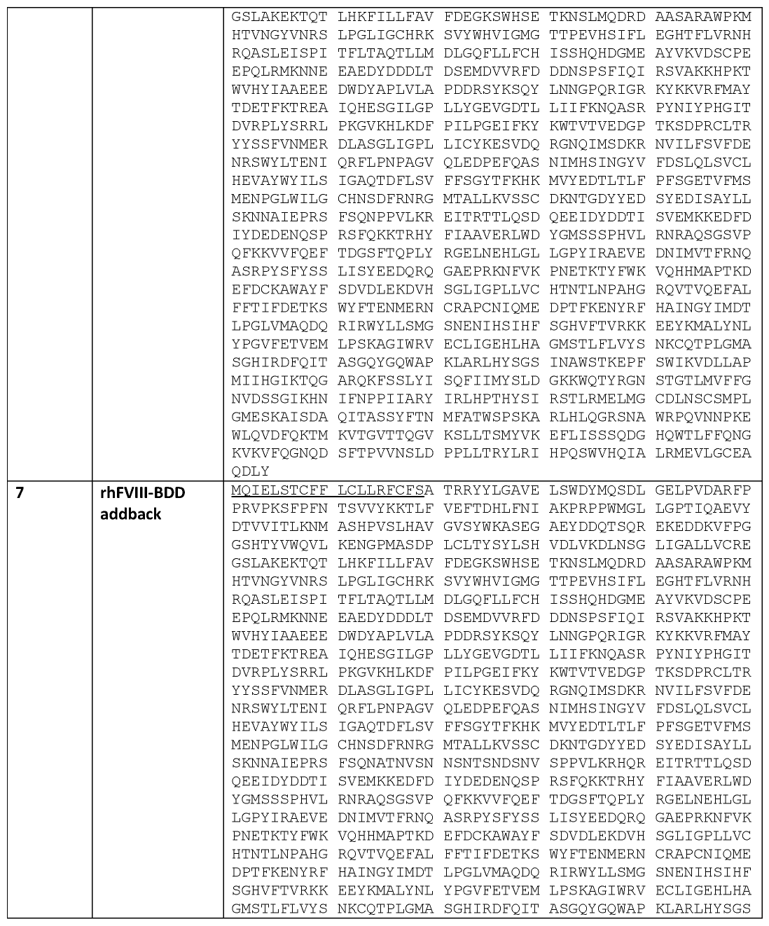

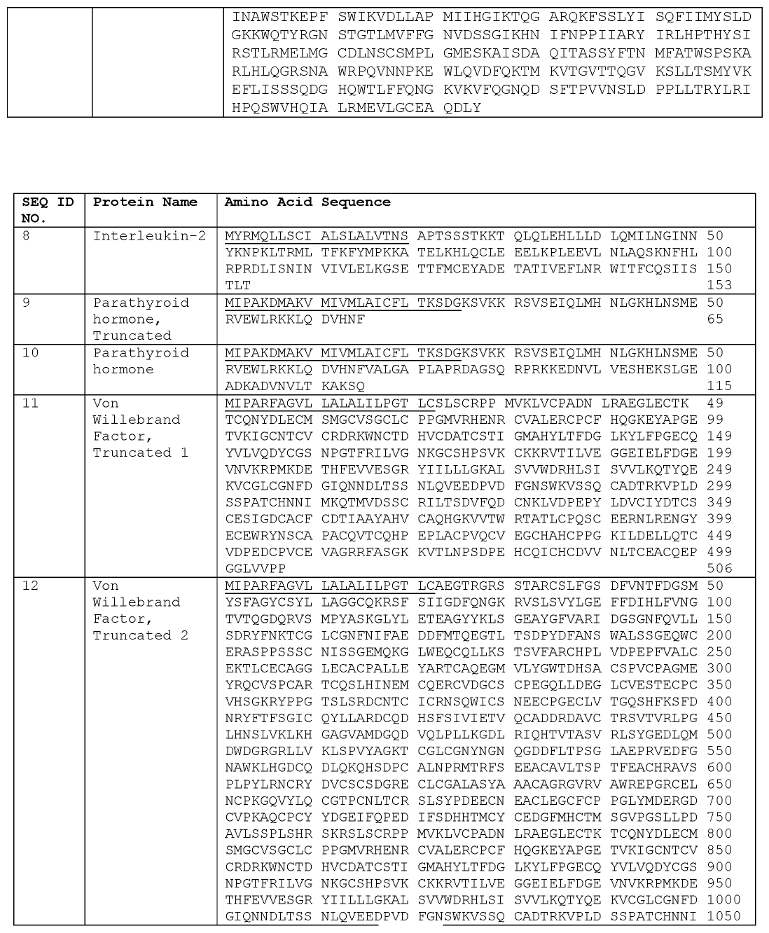

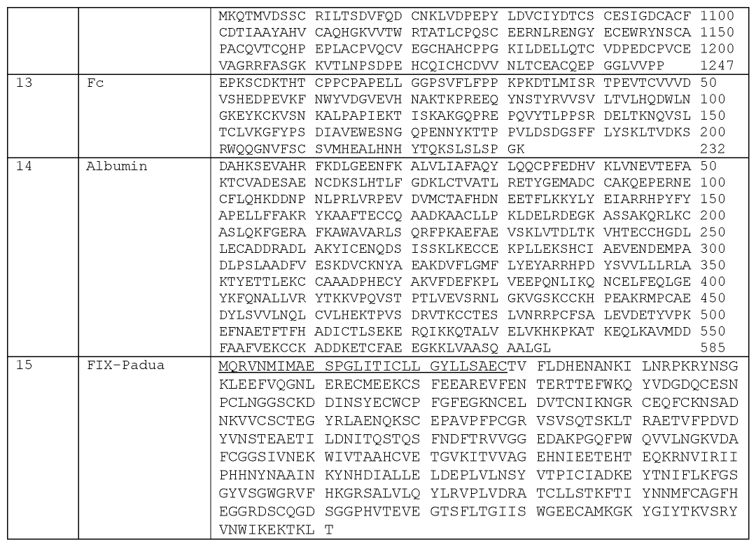

In some embodiments, the MSFC is engineered to express a Factor VIII, e.g., a recombinant Factor VIII. In some embodiments, the MSFC is derived from human tissue and is engineered to express a Factor VIII, e.g., a recombinant Factor VIII. In some embodiments, the recombinant Factor VIII is a B-domain-deleted recombinant Factor VIII (FVIII-BDD). In some embodiments, the engineered MSFC is derived from a human MSC line (e.g., Bone Marrow-Derived Mesenchymal Stem Cells; ATCC PCS-500-012™) and comprises an exogenous nucleic acid sequence which encodes the FVIII-BDD amino acid sequence shown in FIG. 1 (SEQ ID NO:1), or encodes one of the single-chain FVIII-BDD amino acid sequences set forth in SEQ ID NO:3, 4, 5 and 6.

In some embodiments, the MSFC is derived from human tissue and is engineered to express a Factor IX, e.g., a recombinant Factor IX. In some embodiments, the MSFC is engineered to express a Factor IX, e.g., a wild-type human Factor IX (FIX), such as that shown in FIG. 2 (SEQ ID NO:2) or a polymorphic variant thereof (e.g., alanine substituted for threonine at amino acid position 148 of SEQ ID NO:2). In some embodiments, the MSFC is engineered to express a gain-in-function (GIF) variant of a wild-type FIX protein (FIX-GIF), wherein the GIF variant has higher specific activity than the corresponding wild-type FIX. In some embodiments, the MSFC is an engineered MSFC and comprises an exogenous nucleic acid sequence which encodes SEQ ID NO:2, except for having an amino acid substituted for arginine at a position corresponding to amino acid position 338 of SEQ ID NO:2. In some embodiments, the substituting amino acid at a position corresponding to amino acid position 338 of SEQ ID NO:2 is alanine, asparagine, aspartic acid, cysteine, glutamic acid, glutamine, histidine, leucine, lysine, or tyrosine. In some embodiments, the substituting amino acid at a position corresponding to amino acid position 338 of SEQ ID NO:2 is leucine (R338L), and the resulting gain-in-function variant is also known as FIX-Padua.

In some embodiments, the MSFC is engineered to express a truncated variant of vWF, e.g., consisting of domains D1-D3 (e.g., SEQ ID NO:12), or consisting of D′D3 (e.g., SEQ ID NO:11).

In some embodiments, the replacement therapy or replacement protein is an enzyme, e.g., alpha-galactosidase, alpha-L-iduronidase (IDUA), or N-sulfoglucosamine sulfohydrolase (SGSH). In some embodiments, the replacement therapy or replacement protein is an enzyme, e.g., an alpha-galactosidase A (e.g., comprises a naturally-occurring human alpha-galactosidase A amino acid sequence or a variant thereof). In some embodiments, the replacement therapy or replacement protein is a cytokine (e.g., interleukin 2, e.g., SEQ ID NO:8) or an antibody. In some embodiments, the replacement therapy or replacement protein is a parathyroid hormone polypeptide (e.g., SEQ ID NO:9 or SEQ ID NO:10).

In some embodiments, the therapeutic agent is a sugar, e.g., monosaccharide, disaccharide, oligosaccharide, or polysaccharide. In some embodiments, a sugar comprises a triose, tetrose, pentose, hexose, or heptose moiety. In some embodiments, the sugar comprises a a linear monosaccharide or a cyclized monosaccharide. In some embodiments, the sugar comprises a glucose, galactose, fructose, rhamnose, mannose, arabinose, glucosamine, galactosamine, sialic acid, mannosamine, glucuronic acid, galactosuronic acid, mannuronic acid, or guluronic acid moiety. In some embodiments, the sugar is attached to a protein (e.g., an N-linked glycan or an O-linked glycan). Exemplary sugars include glucose, galactose, fructose, mannose, rhamnose, sucrose, ribose, xylose, sialic acid, maltose, amylose, inulin, a fructooligosaccharide, galactooligosaccharide, a mannan, a lectin, a pectin, a starch, cellulose, heparin, hyaluronic acid, chitin, amylopectin, or glycogen. In some embodiments, the therapeutic agent is a sugar alcohol.

In some embodiments, the therapeutic agent is a lipid. A lipid may be hydrophobic or amphiphilic, and may form a tertiary structure such as a liposome, vesicle, or membrane or insert into a liposome, vesicle, or membrane. A lipid may comprise a fatty acid, glycerolipid, glycerophospholipid, sterol lipid, prenol lipid, sphingolipid, saccharolipid, polyketide, or sphingolipid. Examples of lipids produced by the MSFCs described herein include anandamide, docosahexaenoic acid, aprostaglandin, a leukotriene, a thromboxane, an eicosanoid, a triglyceride, a cannabinoid, phosphatidylcholine, phosphatidylethanolamine, a phosphatidylinositol, a phosohatidic acid, a ceramide, a sphingomyelin, a cerebroside, a ganglioside, estrogen, androsterone, testosterone, cholesterol, a carotenoid, a quinone, a hydroquinone, or a ubiquinone.

In some embodiments, the therapeutic agent is a small molecule. A small molecule may include a natural product produced by a cell. In some embodiments, the small molecule has poor availability or does not comply with the Lipinski rule of five (a set of guidelines used to estimate whether a small molecule will likely be an orally active drug in a human; see, e.g., Lipinski, C. A. et al (2001) Adv Drug Deliv 46:2-36). Exemplary small molecule natural products include an anti-bacterial drug (e.g., carumonam, daptomycin, fidaxomicin, fosfomycin, ispamicin, micronomicin sulfate, miocamycin, mupiocin, netilmicin sulfate, teicoplanin, thienamycin, rifamycin, erythromycin, vancomycin), an anti-parasitic drug (e.g., artemisinin, ivermectin), an anticancer drug (e.g., doxorubicin, aclarubicin, aminolaevulinic acid, arglabin, omacetaxine mepesuccinate, paclitaxel, pentostatin, peplomycin, romidepsin, trabectdin, actinomycin D, bleomycin, chromomycin A, daunorubicin, leucovorin, neocarzinostatin, streptozocin, trabectedin, vinblastine, vincristine), anti-diabetic drug (e.g., voglibose), a central nervous system drug (e.g., L-dopa, galantamine, zicontide), a statin (e.g., mevastatin), an anti-fungal drug (e.g., fumagillin, cyclosporin), 1-deoxynojirimycin, and theophylline, sterols (cholesterol, estrogen, testosterone). Additional small molecule natural products are described in Newman, D. J. and Cragg, M. (2016) J Nat Prod 79:629-661 and Butler, M. S. et al (2014) Nat Prod Rep 31:1612-1661, which are incorporated herein by reference in their entirety.

In some embodiments, the MSFC is engineered to synthesize a non-protein or non-peptide small molecule. For example, in an embodiment an MSFC can produce a statin (e.g., taurostatin, pravastatin, fluvastatin, or atorvastatin).

In some embodiments, the therapeutic agent is an antigen (e.g., a viral antigen, a bacterial antigen, a fungal antigen, a plant antigen, an environmental antigen, or a tumor antigen). An antigen is recognized by those skilled in the art as being immunostimulatory, i.e., capable of stimulating an immune response or providing effective immunity to the organism or molecule from which it derives. An antigen may be a nucleic acid, peptide, protein, sugar, lipid, or a combination thereof.

The MSFCs, e.g., engineered MSFCs, e.g., engineered MSFCs described herein, may produce a single therapeutic agent or a plurality of therapeutic agents. In some embodiments, the MSFCs produce a single therapeutic agent. In some embodiments, a cluster of MSFCs comprises MSFCs that produce a single therapeutic agent. In some embodiments, at least about 1%, 5%, 10%, 20%, 25%, 30%, 40%, 50%, 60%, 70%, 80%, 90%, 95%, or 99% of the MSFCs in a cluster produce a single therapeutic agent (e.g., a therapeutic agent described herein). In some embodiments, the MSFCs produce a plurality of therapeutic agents, e.g., at least 2, 3, 4, 5, 6, 7, 8, 9, or 10 therapeutic agents. In some embodiments, a cluster of MSFCs comprises MSFCs that produce a plurality of therapeutic agents. In some embodiments, at least about 1%, 5%, 10%, 20%, 25%, 30%, 40%, 50%, 60%, 70%, 80%, 90%, 95%, or 99% of the MSFCs in a cluster produce a plurality of therapeutic agents (e.g., a therapeutic agent described herein).

The therapeutic agents may be related or may form a complex. In some embodiments, the therapeutic agent secreted or released from an MSFC in an active form. In some embodiments, the therapeutic agent is secreted or released from an MSFC in an inactive form, e.g., as a prodrug. In the latter instance, the therapeutic agent may be activated by a downstream agent, such as an enzyme. In some embodiments, the therapeutic agent is not secreted or released from an MSFC, but is maintained intracellularly. For example, the therapeutic agent may be an enzyme involved in detoxification or metabolism of an unwanted substance, and the detoxification or metabolism of the unwanted substance occurs intracellularly.

### Implantable Elements

The present disclosure comprises MSFCs (e.g., engineered MSFCs, e.g., engineered MSC) entirely or partially disposed with an implantable element. The implantable element may comprise an enclosing element that encapsulates or coats an MSFC, in part or in whole. In an embodiment, an implantable element comprises an enclosing component that is formed, or could be formed, in situ on or surrounding an MSFC, e.g., a plurality of MSFCs, e.g., a cluster of MSFCs, or on a microcarrier, e.g., a bead, or a matrix comprising an MSFC or MSFCs (referred to herein as an “in-situ encapsulated implantable element”).

Exemplary implantable elements and enclosing components comprise materials such as metals, metallic alloys, ceramics, polymers, fibers, inert materials, and combinations thereof. An implantable element may be used to encapsulate an MSFC (e.g., an engineered MSFC, e.g., an engineered MSFC) or a cluster of MSFCs (e.g., engineered MSFCs, e.g., engineered MSFCs). An implantable element may be completely made up of one type of material, or may just refer to a surface or the surface of an implantable element (e.g., the outer surface or an inner surface). In some embodiments, the implantable element is chemically modified, e.g., with a compound described herein.

In some embodiments, the material is a metal or a metallic alloy. Exemplary metallic or metallic alloys include comprising titanium and titanium group alloys (e.g., nitinol, nickel titanium alloys, thermo-memory alloy materials), platinum, platinum group alloys, stainless steel, tantalum, palladium, zirconium, niobium, molybdenum, nickel-chrome, chromium molybdenum alloys, or certain cobalt alloys (e.g., cobalt-chromium and cobalt-chromium-nickel alloys, e.g., ELGILOY® and PHYNOX®). For example, a metallic material may be stainless steel grade 316 (SS 316L) (comprised of Fe, <0.3% C, 16-18.5% Cr, 10-14% Ni, 2-3% Mo, <2% Mn, <1% Si, <0.45% P, and <0.03% S).

In some embodiments, the material is a ceramic. Exemplary ceramic materials include oxides, carbides, or nitrides of the transition elements, such as titanium oxides, hafnium oxides, iridium oxides, chromium oxides, aluminum oxides, and zirconium oxides. Silicon based materials, such as silica, may also be used.

In some embodiments, the material is a polymer. A polymer may be a linear, branched, or cross-linked polymer, or a polymer of selected molecular weight ranges, degree of polymerization, viscosity or melt flow rate. Branched polymers can include one or more of the following types: star polymers, comb polymers, brush polymers, dendronized polymers, ladders, and dendrimers. A polymer may be a thermoresponsive polymer, e.g., gel (e.g., becomes a solid or liquid upon exposure to heat or a certain temperature) or a photocrosslinkable polymers. Exemplary polymers include polystyrene, polyethylene, polypropylene, polyacetylene, poly(vinyl chloride) (PVC), polyolefin copolymers, poly(urethane)s, polyacrylates and polymethacrylates, polyacrylamides and polymethacrylamides, poly(methyl methacrylate), poly(2-hydroxyethyl methacrylate), polyesters, polysiloxanes, polydimethylsiloxane (PDMS), polyethers, poly(orthoester), poly(carbonates), poly(hydroxyalkanoate)s, polyfluorocarbons, PEEK®, Teflon® (polytetrafluoroethylene, PTFE), PEEK, silicones, epoxy resins, Kevlar®, Dacron® (a condensation polymer obtained from ethylene glycol and terephthalic acid), polyethylene glycol, nylon, polyalkenes, phenolic resins, natural and synthetic elastomers, adhesives and sealants, polyolefins, polysulfones, polyacrylonitrile, biopolymers such as polysaccharides and natural latex, collagen, cellulosic polymers (e.g., alkyl celluloses, etc.), polyethylene glycol and 2-hydroxyethyl methacrylate (HEMA), polysaccharides, poly(glycolic acid), poly(L-lactic acid) (PLLA), poly(lactic glycolic acid) (PLGA), a polydioxanone (PDA), or racemic poly(lactic acid), polycarbonates, (e.g., polyamides (e.g., nylon)), fluoroplastics, carbon fiber, agarose, alginate, chitosan, and blends or copolymers thereof.

In some embodiments, the material is a polyethylene. Exemplary polyethylenes include ultra-low-density polyethylene (ULDPE) (e.g., with polymers with densities ranging from 0.890 to 0.905 g/cm³, containing comonomer); very-low-density polyethylene (VLDPE) (e.g., with polymers with densities ranging from 0.905 to 0.915 g/cm³, containing comonomer); linear low-density polyethylene (LLDPE) (e.g., with polymers with densities ranging from 0.915 to 0.935 g/cm³, contains comonomer); low-density polyethylene (LDPE) (e.g., with polymers with densities ranging from about 0.915 to 0.935 g/m³); medium density polyethylene (MDPE) (e.g., with polymers with densities ranging from 0.926 to 0.940 g/cm³, may or may not contain comonomer); high-density polyethylene (HDPE) (e.g., with polymers with densities ranging from 0.940 to 0.970 g/cm³, may or may not contain comonomer).

In some embodiments, the material is a polypropylene. Exemplary polypropylenes include homopolymers, random copolymers (homophasic copolymers), and impact copolymers (heterophasic copolymers), e.g., as described in McKeen, Handbook of Polymer Applications in Medicine and Medical Devices, 3-Plastics Used in Medical Devices, (2014):21-53, which is incorporated herein by reference in its entirety.

In some embodiments, the material is a polystyrene. Exemplary polystyrenes include general purpose or crystal (PS or GPPS), high impact (HIPS), and syndiotactic (SPS) polystyrene.

In some embodiments, the material is a thermoplastic elastomer (TPE). Exemplary TPEs include (i) TPA—polyamide TPE, comprising a block copolymer of alternating hard and soft segments with amide chemical linkages in the hard blocks and ether and/or ester linkages in the soft blocks; (ii) TPC—copolyester TPE, consisting of a block copolymer of alternating hard segments and soft segments, the chemical linkages in the main chain being ester and/or ether; (iii) TPO—olefinic TPE, consisting of a blend of a polyolefin and a conventional rubber, the rubber phase in the blend having little or no cross-linking; (iv) TPS—styrenic TPE, consisting of at least a triblock copolymer of styrene and a specific diene, where the two end blocks (hard blocks) are polystyrene and the internal block (soft block or blocks) is a polydiene or hydrogenated polydiene; (v) TPU—urethane TPE, consisting of a block copolymer of alternating hard and soft segments with urethane chemical linkages in the hard blocks and ether, ester or carbonate linkages or mixtures of them in the soft blocks; (vi) TPV—thermoplastic rubber vulcanizate consisting of a blend of a thermoplastic material and a conventional rubber in which the rubber has been cross-linked by the process of dynamic vulcanization during the blending and mixing step; and (vii) TPZ—unclassified TPE comprising any composition or structure other than those grouped in TPA, TPC, TPO, TPS, TPU, and TPV.

In some embodiments, the material is a polymer, and the polymer is alginate. Alginate is a polysaccharide made up of β-D-mannuronic acid (M) and α-L-guluronic acid (G). In some embodiments, the alginate is a high guluronic acid (G) alginate, and comprises greater than about 50%, 55%, 60%, 65%, 70%, 75%, 80%, 85%, 90%, or more guluronic acid (G). In some embodiments, the alginate is a high mannuronic acid (M) alginate, and comprises greater than about 50%, 55%, 60%, 65%, 70%, 75%, 80%, 85%, 90%, or more mannuronic acid (M). In some embodiments, the ratio of M:G is about 1. In some embodiments, the ratio of M:G is less than 1. In some embodiments, the ratio of M:G is greater than 1.

The polymer may be covalently or non-covalently associated with an enclosing component of the implantable element (e.g., the surface). In some embodiments, the polymer is covalently associated with an enclosing component of the implantable element (e.g., on the inner surface or outer surface of an implantable element). In some embodiments, the polymer is non-covalently associated with an enclosing component of the implantable element (e.g., on the inner surface or outer surface of an implantable element). The polymer can be applied by a variety of techniques in the art including, but not limited to, spraying, wetting, immersing, dipping, such as dip coating (e.g., intraoperative dip coating), painting, or otherwise applying a hydrophobic polymer to a surface of the enclosing component or the implantable element itself.

The MSFCs described herein may be encapsulated or contained, in part or in whole, within an enclosing component or an implantable device comprising a material or a number of components or materials. Exemplary components or materials can be purely structural, therapeutic, or both. An enclosing component or implantable element can comprise a biomolecule component, e.g., a carbohydrate, e.g., a polysaccharide, e.g., a marine polysaccharide, e.g., alginate, agar, agarose, carrageenans, cellulose and amylose, chitin and chitosan; cross-linked polysaccharides, e.g., cross-linked by diacrylates; or a polysaccharide or derivative/modification thereof described in, e.g., Laurienzo (2010), Mar. Drugs. 8.9:2435-65.

In an embodiment, the implantable element comprises an enclosing component that comprises a flexible polymer, e.g., alginate (e.g., a chemically modified alginate), PLA, PLG, PEG, CMC, or mixtures thereof (referred to herein as a “polymer encapsulated implantable device”).

In an embodiment, an implantable element comprises an enclosing component that is formed, or could be formed, in situ on or surrounding an MSFC, e.g., a plurality of MSFCs, e.g., a cluster of MSFCs, or on a microcarrier, e.g., a bead, or a matrix comprising an MSFC or MSFCs (referred to herein as an “in-situ encapsulated implantable element”).

In an embodiment, an implantable element comprises an enclosing component that is preformed prior to combination with the enclosed MSFC, e.g., a plurality of MSFCs, e.g., a cluster of MSFCs, or a microcarrier, e.g., a bead or a matrix comprising an MSFC (referred to herein as device-based-implantable element).

An implantable element can include a protein or polypeptide, e.g., an antibody, protein, enzyme, or growth factor. An implantable element can include an active or inactive fragment of a protein or polypeptide, such as glucose oxidase (e.g., for glucose sensor), kinase, phosphatase, oxygenase, hydrogenase, reductase.

Implantable elements can include any material, such as a material described herein. In some embodiments, an implantable element is made up of one material or many types of materials. In some embodiments, an implantable element comprises a polymer (e.g., hydrogel, plastic) component. Exemplary polymers include polyethylene, polypropylene, polystyrene, polyester (e.g., PLA, PLG, or PGA, polyhydroxyalkanoates (PHAs), or other biosorbable plastic), polycarbonate, polyvinyl chloride (PVC), polyethersulfone (PES), polyacrylate (e.g., acrylic or PMMA), hydrogel (e.g., acrylic polymer or blend of acrylic and silicone polymers), polysulfone, polyetheretherketone, thermoplastic elastomers (TPE or TPU), thermoset elastomer (e.g., silicone (e.g., silicone elastomer)), poly-p-xylylene (Parylene), fluoropolymers (e.g., PTFE), and polyacrylics such as poly(acrylic acid) and/or poly(acrylamide), or mixtures thereof.

Implantable elements can comprise non organic or metal components or materials, e.g., steel (e.g., stainless steel), titanium, other metal or alloy. Implantable elements can include nonmetal components or materials, e.g., ceramic, or hydroxyapatite elements.

Implantable elements can include components or materials that are made of a conductive material (e.g., gold, platinum, palladium, titanium, copper, aluminum, silver, metals, any combinations of these, etc.).

Implantable elements can include more than one component, e.g., more than one component disclosed herein, e.g., more than one of a metal, plastic, ceramic, composite, or hybrid material.

In metal-containing implantable elements, the amount of metal (e.g., by % weight, actual weight) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

In plastic-containing implantable elements, the amount of plastic (e.g., by % weight, actual weight) can be at least 5%, e.g., at least 5%, 10%, 20%, 30%, 40%, 50%, 60%, 70%, 80%, 90%, 95%, 99%, or more, w/w; or less than 20%, e.g., less than 20%, 15%, 10%, 5%, 1%, 0.5%, 0.1%, or less.

In ceramic-containing implantable elements, the amount of ceramic (e.g., by % weight, actual weight) can be at least 5%, e.g., at least 5%, 10%, 20%, 30%, 40%, 50%, 60%, 70%, 80%, 90%, 95%, 99%, or more, w/w; or less than 20%, e.g., less than 20%, 15%, 10%, 5%, 1%, 0.5%, 0.1%, or less.

Implantable elements included herein include implantable elements that are configured with a lumen, e.g., a lumen having one, two or more openings, e.g., tubular devices. A typical stent is an example of a device configured with a lumen and having two openings. Other examples include shunts.

Implantable elements included herein include flexible implantable elements, e.g., that are configured to conform to the shape of the body.

Implantable elements included herein include components that stabilize the location of the implantable element, e.g., an adhesive, or fastener, e.g., a torque-based or friction-based fastener, e.g., a screw or a pin.

Implantable elements included herein may be configured to monitor a substance, e.g., an exogenous substance, e.g., a therapeutic agent or toxin, or an endogenous body product, e.g., insulin. In some embodiments, the implantable element is a diagnostic.

Implantable elements included herein may be configured to release a substance, e.g., an exogenous substance, e.g., a therapeutic agent described herein. In some embodiments, the therapeutic agent is a compound of Formula (I) or a pharmaceutically acceptable salt thereof. In some embodiments, the therapeutic agent is a biological material. In some embodiments, the therapeutic agent is a nucleic acid (e.g., an RNA or DNA), protein (e.g., a hormone, enzyme, antibody, antibody fragment, antigen, or epitope), small molecule, lipid, drug, vaccine, or any derivative thereof.

Implantable elements herein may be configured to change conformation in response to a signal or movement of the body, e.g., an artificial joint, e.g., a knee, hip, or other artificial joint.

Exemplary implantable elements include a stent, shunt, dressing, ocular device, port, sensor, orthopedic fixation device, implant (e.g., a dental implant, ocular implant, silicon implant, corneal implant, dermal implant, intragastric implant, facial implant, hip implant, bone implant, cochlear implant, penile implant, implants for control of incontinence), skin covering device, dialysis media, drug-delivery device, artificial or engineered organ (e.g., a spleen, kidney, liver, or heart), drainage device (e.g., a bladder drainage device), cell selection system, adhesive (e.g., a cement, clamp, clip), contraceptive device, intrauterine device, defibrillator, dosimeter, electrode, pump (e.g., infusion pump) filter, embolization device, fastener, fillers, fixative, graft, hearing aid, cardio or heart-related device (e.g., pacemaker, heart valve), battery or power source, hemostatic agent, incontinence device, intervertebral body fusion device, intraoral device, lens, mesh, needle, nervous system stimulator, patch, peritoneal access device, plate, plug, pressure monitoring device, ring, transponder, and valve. Also included are devices used in one or more of anesthesiology, cardiology, clinical chemistry, otolaryngology, dentistry, gastroenterology, urology, hematology, immunology, microbiology, neurology, obstetrics/gynecology, ophthalmology, orthopedic, pathology, physical medicine, radiology, general or plastic surgery, veterinary medicine, psychiatry, surgery, and/or clinical toxicology.

Implantable elements included herein include FDA class 1, 2, or 3 devices, e.g., devices that are unclassified or not classified, or classified as a humanitarian use device (HUD).

In some embodiments, an implantable element includes encapsulated or entrapped cells or tissues. The cells or tissue can be encapsulated or entrapped in a polymer. In some embodiments, an implantable element includes an MSFC, e.g., an MSFC disposed within a polymeric enclosing component (e.g., alginate).

In some embodiments, an implantable element targets or is designed for a certain system of the body, e.g. the nervous system (e.g., peripheral nervous system (PNS) or central nervous system (CNS)), vascular system, skeletal system, respiratory system, endocrine system, lymph system, reproductive system, or gastrointestinal tract. In some embodiments, an implantable element is targeted to the CNS. In some embodiments, an implantable element targets or is designed for a certain part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis.

### Features of Implantable Elements

Components or materials used in an implantable element (or the entire implantable element) can be optimized for one or more of biocompatibility (e.g., it minimizes immune rejection or fibrosis; heat-resistance; elasticity; tensile strength; chemical resistance (e.g., resistance to oils, greases, disinfectants, bleaches, processing aids, or other chemicals used in the production, use, cleaning, sterilizing and disinfecting of the device); electrical properties; surface and volume conductivity or resistivity, dielectric strength; comparative tracking index; mechanical properties; shelf life, long term durability sterilization capability (e.g., capable of withstanding sterilization processes, such as steam, dry heat, ethylene oxide (EtO), electron beam, and/or gamma radiation, e.g., while maintaining the properties for the intended use of the device), e.g., thermal resistance to autoclave/steam conditions, hydrolytic stability for steam sterilization, chemical resistance to EtO, resistance to high-energy radiation (e.g., electron beam, UV, and gamma); or crystal structure.

An implantable element can be assembled in vivo (e.g., injectable substance that forms a structured shape in vivo, e.g., at body temperature) or ex vivo.

An implantable element can have nanodimensions, e.g., can comprise a nanoparticle, e.g., nanoparticle made of a polymer described herein, e.g., PLA. Nanoparticles can be chemically modified nanoparticles, e.g., modified to prevent uptake by macrophages and Kupfer cells (e.g., a process called opsonization); or to alter the circulation half-life of the nanoparticle. Nanoparticles can include iron nanoparticle (injectable) (e.g., Advanced Magnetics iron nanoparticles). Exemplary nanoparticles are described in Veiseh et al (2010) Adv Drug Deliv Rev 62:284-304, which is incorporated herein by reference in its entirety.

An implantable element can be configured for implantation, or implanted or disposed into or onto any site of the body. In some embodiments, an implantable element is configured for implantation, implanted or disposed into the omentum of a subject, into the subcutaneous fat of a subject, or into the muscle tissue of a subject. An implantable element can be configured for implantation, or implanted, or disposed on or in the skin; a mucosal surface, a body cavity, the peritoneal cavity (e.g., the lesser sac); the central nervous system, e.g., the brain or spinal cord; an organ, e.g., the heart, liver, kidney, spleen, lung, lymphatic system, vasculature, the oral cavity, the nasal cavity, the teeth, the gums, the GI tract; bone; hip; fat tissue; muscle tissue; circulating blood; the eye (e.g., intraocular); breast, vagina; uterus, a joint, e.g., the knee or hip joint, or the spine. In some embodiments, the implantable element is configured for implantation or implanted or disposed into the peritoneal cavity (e.g., the lesser sac).

In some embodiments, the implantable element is configured for implantation or implanted or disposed into the peritoneal cavity (e.g., the omentum). In some embodiments, the implantable element is configured for implantation or implanted or disposed into or onto the lesser sac, also known as the omental bursa or bursalis omentum. The lesser sac refers to a cavity located in the abdomen formed by the omentum, and is in close proximity to, for example, the greater omentum, lesser omentum, stomach, small intestine, large intestine, liver, spleen, gastrosplenic ligament, adrenal glands, and pancreas. Typically, the lesser sac is connected to the greater sac via the omental foramen (i.e., the Foramen of Winslow). In some embodiments, the lesser sac comprises a high concentration of adipose tissue. An implantable element may be implanted in the peritoneal cavity (e.g., the omentum, e.g., the lesser sac) or disposed on a surface within the peritoneal cavity (e.g., omentum, e.g., lesser sac) via injection or catheter. Additional considerations for implantation or disposition of an implantable element into the omentum (e.g., the lesser sac) are provided in M. Pellicciaro et al. (2017) CellR4 5(3):e2410, which is incorporated herein by reference in its entirety.

In some embodiments, the implantable element is configured for implantation or implanted or disposed into the central nervous system (CNS), e.g., the brain or spinal cord and their corresponding tissues and cavities. In vertebrates, the CNS is contained within the dorsal body cavity, including the cranial cavity and the spinal canal. In some embodiments, the implantable element is configured for implantation or implanted or disposed into an intracerebral space, e.g., the intraparenchymal space, the intraventricular space, or the subdural space. An implantable element may be implanted in the CNS or disposed on a surface within the CNS through a hole made in the skull and delivered via injection or catheter.

In some embodiments, the implantable element is configured for implantation or implanted in or disposed into the eye. Exemplary regions suitable for implantation or disposition of the implantable element include any surface or cavity within the eye, such as the retina, cornea, epithelium, aqueous humor, or vitreal space. In some embodiments, the implantable element is configured for implantation or implanted or disposed into the vitreal space. An implantable element may be implanted in the eye or disposed on a surface within the eye through incision and/or injection.

An implantable element can comprise an electrochemical sensor, e.g., an electrochemical sensor including a working electrode and a reference electrode. For example, an electrochemical sensor includes a working electrode and a reference electrode that reacts with an analyte to generate a sensor measurement related to a concentration of the analyte in a fluid to which the eye-mountable device is exposed. The implantable element can comprise a window, e.g., of a transparent polymeric material having a concave surface and a convex surface a substrate, e.g., at least partially embedded in a transparent polymeric material. An implantable element can also comprise an electronics module including one or more of an antenna; and a controller electrically connected to the electrochemical sensor and the antenna, wherein the controller is configured to control the electrochemical sensor to obtain a sensor measurement related to a concentration of an analyte in a fluid to which the implantable element, e.g., an mountable implantable element is exposed and use the antenna to indicate the sensor measurement.

In some embodiments, an implantable element has a mean diameter or size that is greater than 1 mm, preferably 1.5 mm or greater. In some embodiments, an implantable element can be as large as 8 mm in diameter or size. For example, an implantable element described herein is in a size range of 1 mm to 8 mm, 1 mm to 6 mm, 1 mm to 5 mm, 1 mm to 4 mm, 1 mm to 3 mm, 1 mm to 2 mm, 1 mm to 1.5 mm, 1.5 mm to 8 mm, 1.5 mm to 6 mm, 1.5 mm to 5 mm, 1.5 mm to 4 mm, 1.5 mm to 3 mm, 1.5 mm to 2 mm, 2 mm to 8 mm, 2 mm to 7 mm, 2 mm to 6 mm, 2 mm to 5 mm, 2 mm to 4 mm, 2 mm to 3 mm, 2.5 mm to 8 mm, 2.5 mm to 7 mm, 2.5 mm to 6 mm, 2.5 mm to 5 mm, 2.5 mm to 4 mm, 2.5 mm to 3 mm, 3 mm to 8 mm, 3 mm to 7 mm, 3 mm to 6 mm, 3 mm to 5 mm, 3 mm to 4 mm, 3.5 mm to 8 mm, 3.5 mm to 7 mm, 3.5 mm to 6 mm, 3.5 mm to 5 mm, 3.5 mm to 4 mm, 4 mm to 8 mm, 4 mm to 7 mm, 4 mm to 6 mm, 4 mm to 5 mm, 4.5 mm to 8 mm, 4.5 mm to 7 mm, 4.5 mm to 6 mm, 4.5 mm to 5 mm, 5 mm to 8 mm, 5 mm to 7 mm, 5 mm to 6 mm, 5.5 mm to 8 mm, 5.5 mm to 7 mm, 5.5 mm to 6 mm, 6 mm to 8 mm, 6 mm to 7 mm, 6.5 mm to 8 mm, 6.5 mm to 7 mm, 7 mm to 8 mm, or 7.5 mm to 8 mm. In some embodiments, the implantable element has a mean diameter or size between 1 mm to 8 mm. In some embodiments, the implantable element has a mean diameter or size between 1 mm to 4 mm. In some embodiments, the implantable element has a mean diameter or size between 1 mm to 2 mm.

In some embodiments, an implantable element comprises at least one pore or opening, e.g., to allow for the free flow of materials. In some embodiments, the mean pore size of an implantable element is between about 0.1 μm to about 10 μm. For example, the mean pore size may be between 0.1 μm to 10 μm, 0.1 μm to 5 μm, 0.1 μm to 2 μm, 0.15 μm to 10 μm, 0.15 μm to 5 μm, 0.15 μm to 2 μm, 0.2 μm to 10 μm, 0.2 μm to 5 μm, 0.25 μm to 10 μm, 0.25 μm to 5 μm, 0.5 μm to 10 μm, 0.75 μm to 10 μm, 1 μm to 10 μm, 1 μm to 5 μm, 1 μm to 2 μm, 2 μm to 10 μm, 2 μm to 5 μm, or 5 μm to 10 μm. In some embodiments, the mean pore size of an implantable element is between about 0.1 μm to 10 μm. In some embodiments, the mean pore size of an implantable element is between about 0.1 μm to 5 μm. In some embodiments, the mean pore size of an implantable element is between about 0.1 μm to 1 μm.

In some embodiments, an implantable element is capable of preventing materials over a certain size from passing through a pore or opening. In some embodiments, an implantable element is capable of preventing materials greater than 50 kD, 75 kD, 100 kD, 125 kD, 150 kD, 175 kD, 200 kD, 250 kD, 300 kD, 400 kD, 500 kD, 750 kD, 1,000 kD from passing through.

An implantable element (e.g., an implantable element described herein) may be provided as a preparation or composition for implantation or administration to a subject. In some embodiments, at least 20%, 30%, 35%, 40%, 45%, 50%, 55%, 60%, 65%, 70%, 75%, 80%, 85%, 90%, 95% or 100% of the implantable elements in a preparation or composition have a characteristic as described herein, e.g., mean pore size.

In some embodiments, an implantable element may be used for varying periods of time, ranging from a few minutes to several years. For example, an implantable element may be used from about 1 hour to about 10 years. In some embodiments, an implantable element is used for longer than about 1 hour, 2 hours, 4 hours, 8 hours, 16 hours, 1 day, 48 hours, 2 days, 3 days, 4 days, 5 days, 6 days, 1 week, 2 weeks, 1 month, 2 months, 3 months, 4 months, 5 months, 6 months, 8 months, 10 months, 1 year, 18 months, 2 years, 3 years, 4 years, 5 years, 6 years, 7 years, 8 years, 9 years, 10 years, or more. An implantable element may be configured for the duration of implantation, e.g., configured to resist fibrotic inactivation by fibrosis for all or part of the expected duration.

In some embodiments, the implantable element is easily retrievable from a subject, e.g., without causing injury to the subject or without causing significant disruption of the surrounding tissue. In an embodiment, the implantable element can be retrieved with minimal or no surgical separation of the implantable element from surrounding tissue, e.g., via minimally invasive surgical insection, extraction, or resection.

An implantable element can be configured for limited exposure (e.g., less than 2 days, e.g., less than 2 days, 1 day, 24 hours, 20 hours, 16 hours, 12 hours, 10 hours, 8 hours, 6 hours, 5 hours, 4 hours, 3 hours, 2 hours, 1 hour or less). An implantable element can be configured for prolonged exposure (e.g., at least 2 days, 3 days, 4 days, 5 days, 6 days, 7 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 1.5 years, 2 years, 2.5 years, 3 years, 3.5 years, 4 years or more) An implantable element can be configured for permanent exposure (e.g., at least 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 1.5 years, 2 years, 2.5 years, 3 years, 3.5 years, 4 years or more).

In some embodiments, the implantable element is not an implantable element disclosed in any of WO2012/112982, WO2012/167223, WO2014/153126, WO2016/019391, US2012-0213708, US 2016-0030359, and US 2016-0030360.

In an embodiment, the implantable element comprises an MSFC (e.g., an MSC) described herein. In an embodiment, the implantable element comprises an MSFC, as well as another cell, e.g., a recombinant cell or stem cell, which provides a substance, e.g., a therapeutic agent described therein.

In an embodiment, the MSFC is a human MSFC (e.g., a human MSC), or a cell derived therefrom, and the polypeptide is a human polypeptide. In an embodiment, the MSFC provides a substance that alleviates a disease, disorder, or condition (e.g., as described herein).

### Chemical Modification of Implantable Elements

The present disclosure features an implantable element comprising an MSFC, wherein the implantable element is chemically modified. The chemical modification may impart an improved property to the implantable element when administered to a subject, e.g., modulation of the immune response in the subject, compared with an unmodified implantable element.

In some embodiments, a surface of the implantable element comprising an engineered MSFC (e.g., an engineered MSC) is chemically modified with a compound. In some embodiments, a surface comprises an outer surface or an inner surface of the implantable element. In some embodiments, the surface (e.g., outer surface) of the implantable element comprising an engineered MSFC (e.g., an engineered MSC) is chemically modified with a compound. In some embodiments, the surface (e.g., outer surface) is covalently linked to a compound. In some embodiments, the compound comprises at least one heteroaryl moiety.

In some embodiments, the compound is a compound of Formula (I):

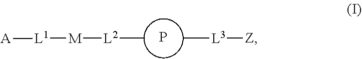

or a pharmaceutically acceptable salt thereof, wherein:

A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₂-C₆-alkenylene)-, —N(R^{C})N(R^{D})—, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, wherein each alkyl, alkenyl, alkynyl, alkylene, alkenylene, heteroalkyl, cycloalkyl, heterocyclyl, aryl, and heteroaryl is linked to an attachment group (e.g., an attachment group defined herein) and is optionally substituted by one or more R¹;

each of L¹ and L³ is independently a bond, alkyl, or heteroalkyl, wherein each alkyl and heteroalkyl is optionally substituted by one or more R²;

L² is a bond;

M is absent, alkyl, heteroalkyl, cycloalkyl, heterocyclyl, aryl, or heteroaryl, each of which is optionally substituted by one or more R³;

P is absent, cycloalkyl, heterocyclyl, or heteroaryl, each of which is optionally substituted by one or more R⁴;

Z is hydrogen, alkyl, alkenyl, alkynyl, heteroalkyl, —OR^{A}, —C(O)R^{A}, —C(O)OR^{A}, —C(O)N(R^{C})(R^{D}), —N(R^{C})C(O)R^{A}, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵;

each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;

or R^{C} and R^{D}, taken together with the nitrogen atom to which they are attached, form a ring (e.g., a 5-7 membered ring), optionally substituted with one or more R⁶;

each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;

each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;

each R⁷ is independently alkyl, alkenyl, alkynyl, heteroalkyl, halogen, cyano, oxo, hydroxyl, cycloalkyl, or heterocyclyl;

x is 1 or 2; and

y is 2, 3, or 4.

In some embodiments, the compound of Formula (I) is a compound of Formula (I-a):

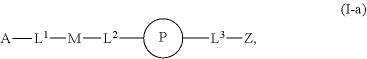

or a pharmaceutically acceptable salt thereof, wherein:

A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₂-C₆-alkenylene)-, —N(R^{C})N(R^{D})—, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, wherein each alkyl, alkenyl, alkynyl, alkylene, alkenylene, heteroalkyl, cycloalkyl, heterocyclyl, aryl, and heteroaryl is linked to an attachment group (e.g., an attachment group defined herein) and is optionally substituted by one or more R¹;

each of L¹ and L³ is independently a bond, alkyl, or heteroalkyl, wherein each alkyl and heteroalkyl is optionally substituted by one or more R²;

L² is a bond;

M is absent, alkyl, heteroalkyl, cycloalkyl, heterocyclyl, aryl, or heteroaryl, each of which is optionally substituted by one or more R³;

P is heteroaryl optionally substituted by one or more R⁴;

Z is alkyl, alkenyl, alkynyl, heteroalkyl, cycloalkyl, heterocyclyl, aryl, or heteroaryl, each of which is optionally substituted by one or more R⁵;

each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;

or R^{C} and R^{D}, taken together with the nitrogen atom to which they are attached, form a ring (e.g., a 5-7 membered ring), optionally substituted with one or more R⁶;

each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y} cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;

each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;

each R⁷ is independently alkyl, alkenyl, alkynyl, heteroalkyl, halogen, cyano, oxo, hydroxyl, cycloalkyl, or heterocyclyl;

x is 1 or 2; and

y is 2, 3, or 4.

In some embodiments, for Formulas (I) and (I-a), A is alkyl, alkenyl, alkynyl, heteroalkyl, cycloalkyl, heterocyclyl, aryl, heteroaryl, —O—, —C(O)O—, —C(O)—, —OC(O)—, —N(R^{C})C(O)—, —N(R^{C})C(O)(C₁-C₆-alkylene)-, —N(R^{C})C(O)(C₂-C₆-alkenylene)-, or —N(R^{C})—. In some embodiments, A is alkyl, alkenyl, alkynyl, heteroalkyl, cycloalkyl, heterocyclyl, aryl, heteroaryl, —O—, —C(O)O—, —C(O)—, —OC(O)—, or —N(R^{C})—. In some embodiments, A is alkyl, alkenyl, alkynyl, heteroalkyl, —O—, —C(O)O—, —C(O)—, —OC(O)—, or —N(R^{C})—. In some embodiments, A is alkyl, —O—, —C(O)O—, —C(O)—, —OC(O), or —N(R^{C})—. In some embodiments, A is —N(R^{C})C(O)—, —N(R^{C})C(O)(C₁-C₆-alkylene)-, or —N(R^{C})C(O)(C₂-C₆-alkenylene)-. In some embodiments, A is —N(R^{C})—. In some embodiments, A is —N(R^{C})—, and R^{C} an R^{D} is independently hydrogen or alkyl. In some embodiments, A is —NH—. In some embodiments, A is —N(R^{C})C(O)(C₁-C₆-alkylene)-, wherein alkylene is substituted with R¹. In some embodiments, A is —N(R^{C})C(O)(C₁-C₆-alkylene)-, and R⁴ is alkyl (e.g., methyl). In some embodiments, A is —NHC(O)C(CH₃)₂—. In some embodiments, A is —N(R^{C})C(O)(methylene)-, and R⁴ is alkyl (e.g., methyl). In some embodiments, A is —NHC(O)CH(CH₃)—. In some embodiments, A is —NHC(O)C(CH₃)—.

In some embodiments, for Formulas (I) and (I-a), L¹ is a bond, alkyl, or heteroalkyl. In some embodiments, L¹ is a bond or alkyl. In some embodiments, L¹ is a bond. In some embodiments, L¹ is alkyl. In some embodiments, L¹ is C₁-C₆ alkyl. In some embodiments, L¹ is —CH₂—, —CH(CH₃)—, —CH₂CH₂CH₂, or —CH₂CH₂—. In some embodiments, L¹ is —CH₂— or —CH₂CH₂—.

In some embodiments, for Formulas (I) and (I-a), L³ is a bond, alkyl, or heteroalkyl. In some embodiments, L³ is a bond. In some embodiments, L³ is alkyl. In some embodiments, L³ is C₁-C₁₂ alkyl. In some embodiments, L³ is C₁-C₆ alkyl. In some embodiments, L³ is —CH₂—. In some embodiments, L³ is heteroalkyl. In some embodiments, L³ is C₁-C₁₂ heteroalkyl, optionally substituted with one or more R² (e.g., oxo). In some embodiments, L³ is C₁-C₆ heteroalkyl, optionally substituted with one or more R² (e.g., oxo). In some embodiments, L³ is —C(O)OCH₂—, —CH₂(OCH₂CH₂)₂—, —CH₂(OCH₂CH₂)₃—, CH₂CH₂O—, or —CH₂O—. In some embodiments, L³ is —CH₂O—.

In some embodiments, for Formulas (I) and (I-a), M is absent, alkyl, heteroalkyl, aryl, or heteroaryl. In some embodiments, M is heteroalkyl, aryl, or heteroaryl. In some embodiments, M is absent. In some embodiments, M is alkyl (e.g., C₁-C₆ alkyl). In some embodiments, M is —CH₂—. In some embodiments, M is heteroalkyl (e.g., C₁-C₆ heteroalkyl). In some embodiments, M is (—OCH₂CH₂—)z, wherein z is an integer selected from 1 to 10. In some embodiments, z is an integer selected from 1 to 5. In some embodiments, M is —OCH₂CH₂—, (—OCH₂CH₂—)₂, (—OCH₂CH₂—)₃, (—OCH₂CH₂—)₄, or (—OCH₂CH₂—)₅. In some embodiments, M is —OCH₂CH₂—, (—OCH₂CH₂—)₂, (—OCH₂CH₂—)₃, or (—OCH₂CH₂—)₄. In some embodiments, M is (—OCH₂CH₂—)₃. In some embodiments, M is aryl. In some embodiments, M is phenyl. In some embodiments, M is unsubstituted phenyl. In some embodiments, M is

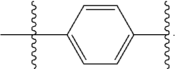 [SMILES: *c1ccc(*)cc1]

In some embodiments, M is phenyl substituted with R⁷ (e.g., 1 R⁷). In some embodiments, M is

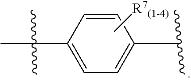

In some embodiments, R⁷ is CF₃.

In some embodiments, for Formulas (I) and (I-a), P is absent, heterocyclyl, or heteroaryl. In some embodiments, P is absent. In some embodiments, for Formulas (I) and (I-a), P is a tricyclic, bicyclic, or monocyclic heteroaryl. In some embodiments, P is a monocyclic heteroaryl. In some embodiments, P is a nitrogen-containing heteroaryl. In some embodiments, P is a monocyclic, nitrogen-containing heteroaryl. In some embodiments, P is a 5-membered heteroaryl. In some embodiments, P is a 5-membered nitrogen-containing heteroaryl. In some embodiments, P is tetrazolyl, imidazolyl, pyrazolyl, or triazolyl, pyrrolyl, oxazolyl, or thiazolyl. In some embodiments, P is tetrazolyl, imidazolyl, pyrazolyl, or triazolyl, or pyrrolyl. In some embodiments, P is imidazolyl. In some embodiments, P is

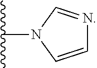 [SMILES: *n1ccnc1]

In some embodiments, P is triazolyl. In some embodiments, P is 1,2,3-triazolyl. In some embodiments, P is

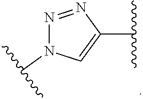 [SMILES: *c1cn(*)nn1]

In some embodiments, P is heterocyclyl. In some embodiments, P is a 5-membered heterocyclyl or a 6-membered heterocyclyl. In some embodiments, P is imidazolidinonyl. In some embodiments, P is

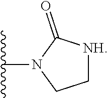 [SMILES: *N1CCNC1=O]

In some embodiments, P is thiomorpholinyl-1,1-dioxidyl. In some embodiments, P is

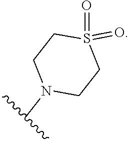 [SMILES: *N1CCS(=O)(=O)CC1]

In some embodiments, for Formulas (I) and (I-a), Z is alkyl, heteroalkyl, cycloalkyl, heterocyclyl, aryl, or heteroaryl. In some embodiments, Z is heterocyclyl. In some embodiments, Z is monocyclic or bicyclic heterocyclyl. In some embodiments, Z is an oxygen-containing heterocyclyl. In some embodiments, Z is a 4-membered heterocyclyl, 5-membered heterocyclyl, or 6-membered heterocyclyl. In some embodiments, Z is a 6-membered heterocyclyl. In some embodiments, Z is a 6-membered oxygen-containing heterocyclyl. In some embodiments, Z is tetrahydropyranyl. In some embodiments, Z is

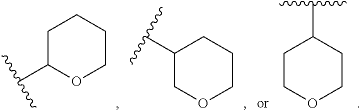

In some embodiments, Z is a 4-membered oxygen-containing heterocyclyl. In some embodiments, Z is

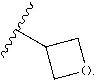 [SMILES: *C1COC1]

In some embodiments, Z is a bicyclic oxygen-containing heterocyclyl. In some embodiments, Z is phthalic anhydridyl. In some embodiments, Z is a sulfur-containing heterocyclyl. In some embodiments, Z is a 6-membered sulfur-containing heterocyclyl. In some embodiments, Z is a 6-membered heterocyclyl containing a nitrogen atom and a sulfur atom. In some embodiments, Z is thiomorpholinyl-1,1-dioxidyl. In some embodiments, Z is

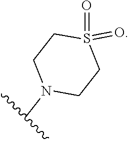 [SMILES: *N1CCS(=O)(=O)CC1]

In some embodiments, Z is a nitrogen-containing heterocyclyl. In some embodiments, Z is a 6-membered nitrogen-containing heterocyclyl. In some embodiments, Z is

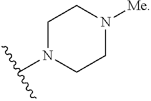 [SMILES: *N1CCN(C)CC1]

In some embodiments, Z is a bicyclic heterocyclyl. In some embodiments, Z is a bicyclic nitrogen-containing heterocyclyl, optionally substituted with one or more R⁵. In some embodiments, Z is 2-oxa-7-azaspiro[3.5]nonanyl. In some embodiments, Z is

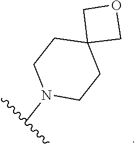 [SMILES: *N1CCC2(CC1)COC2]

In some embodiments, Z is 1-oxa-3,8-diazaspiro[4.5]decan-2-one. In some embodiments, Z is

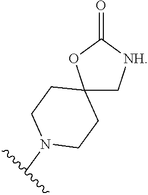 [SMILES: *N1CCC2(CC1)CNC(=O)O2]

In some embodiments, for Formulas (I) and (I-a), Z is aryl. In some embodiments, Z is monocyclic aryl. In some embodiments, Z is phenyl. In some embodiments, Z is monosubstituted phenyl (e.g., with 1 R⁵). In some embodiments, Z is monosubstituted phenyl, wherein the 1 R⁵ is a nitrogen-containing group. In some embodiments, Z is monosubstituted phenyl, wherein the 1 R⁵ is NH₂. In some embodiments, Z is monosubstituted phenyl, wherein the 1 R⁵ is an oxygen-containing group. In some embodiments, Z is monosubstituted phenyl, wherein the 1 R⁵ is an oxygen-containing heteroalkyl. In some embodiments, Z is monosubstituted phenyl, wherein the 1 R⁵ is OCH₃. In some embodiments, Z is monosubstituted phenyl, wherein the 1 R⁵ is in the ortho position. In some embodiments, Z is monosubstituted phenyl, wherein the 1 R⁵ is in the meta position. In some embodiments, Z is monosubstituted phenyl, wherein the 1 R⁵ is in the para position.

In some embodiments, for Formulas (I) and (I-a), Z is alkyl. In some embodiments, Z is C₁-C₁₂ alkyl. In some embodiments, Z is C₁-C₁₀ alkyl. In some embodiments, Z is C₁-C₈ alkyl. In some embodiments, Z is C₁-C₈ alkyl substituted with 1-5 R⁵. In some embodiments, Z is C₁-C₈ alkyl substituted with 1 R⁵. In some embodiments, Z is C₁-C₈ alkyl substituted with 1 R⁵, wherein R⁵ is alkyl, heteroalkyl, halogen, oxo, —OR^{A1}, —C(O)OR^{A1}, —C(O)R^{B1}, —OC(O)R^{B1}, or —N(R^{C1})(R^{D1}). In some embodiments, Z is C₁-C₈ alkyl substituted with 1 R⁵, wherein R⁵ is —OR^{A1}) or —C(O)OR^{A1}. In some embodiments, Z is C₁-C₈ alkyl substituted with 1 R⁵, wherein R⁵ is —OR^{A1} or —C(O)OH. In some embodiments, Z is —CH₃.

In some embodiments, for Formulas (I) and (I-a), Z is heteroalkyl. In some embodiments, Z is C₁-C₁₂ heteroalkyl. In some embodiments, Z is C₁-C₁₀ heteroalkyl. In some embodiments, Z is C₁-C₈ heteroalkyl. In some embodiments, Z is C₁-C₆ heteroalkyl. In some embodiments, Z is a nitrogen-containing heteroalkyl optionally substituted with one or more R⁵. In some embodiments, Z is a nitrogen and sulfur-containing heteroalkyl substituted with 1-5 R⁵. In some embodiments, Z is N-methyl-2-(methylsulfonyl)ethan-1-aminyl.

In some embodiments, Z is —OR^{A} or —C(O)OR^{A}. In some embodiments, Z is —OR^{A} (e.g., —OH or —OCH₃). In some embodiments, Z is —OCH₃. In some embodiments, Z is —C(O)OR^{A} (e.g., —C(O)OH).

In some embodiments, Z is hydrogen.

In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound of Formula (I) is a compound of Formula (I-b):

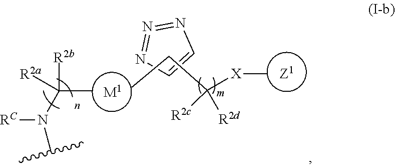

or a pharmaceutically acceptable salt thereof, wherein Ring M¹ is cycloalkyl, heterocyclyl, aryl, or heteroaryl, each of which is optionally substituted with 1-5 R³; Ring Z¹ is cycloalkyl, heterocyclyl, aryl or heteroaryl, optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl, or each of R^{2a} and R^{2b} or R^{2c} and R^{2d} is taken together to form an oxo group; X is absent, N(R¹⁰)(R¹¹), O, or S; R^{C} is hydrogen, alkyl, alkenyl, alkynyl, heteroalkyl, cycloalkyl, heterocyclyl, aryl, or heteroaryl, wherein each of alkyl, alkenyl, alkynyl, heteroalkyl, cycloalkyl, heterocyclyl, aryl, or heteroaryl is optionally substituted with 1-6 R⁶; each R³, R⁵, and R⁶ is independently alkyl, alkenyl, alkynyl, heteroalkyl, halogen, cyano, azido, oxo, —OR^{A1}, —C(O)OR^{A1}, —COR^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each of R¹⁰ and R¹¹ is independently hydrogen, alkyl, alkenyl, alkynyl, heteroalkyl, —C(O)OR^{A1}, —C(O)R^{B1}, —OC(O)R^{B1}, —C(O)N(R^{C1}), cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; each R⁷ is independently alkyl, alkenyl, alkynyl, heteroalkyl, halogen, cyano, oxo, hydroxyl, cycloalkyl, or heterocyclyl; each m and n is independently 1, 2, 3, 4, 5, or 6; and “ [symbol] ” refers to a connection to an attachment group or a polymer described herein. In some embodiments, for each R³ and R⁵, each alkyl, alkenyl, alkynyl, heteroalkyl, cycloalkyl, heterocyclyl, aryl, or heteroaryl is optionally and independently substituted with halogen, oxo, cyano, cycloalkyl, or heterocyclyl.

In some embodiments, the compound of Formula (I-b) is a compound of Formula (Ib-i):

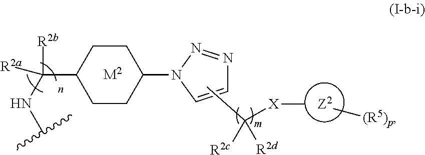

or a pharmaceutically acceptable salt thereof, wherein Ring M² is aryl or heteroaryl optionally substituted with one or more R³; Ring Z² is cycloalkyl, heterocyclyl, aryl, or heteroaryl; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; X is absent, O, or S; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; or two R⁵ are taken together to form a 5-6 membered ring fused to Ring Z²; each R^{A1} and R^{B1} is independently hydrogen, alkyl, or heteroalkyl; m and n are each independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I-b-i) is a compound of Formula (I-b-ii):

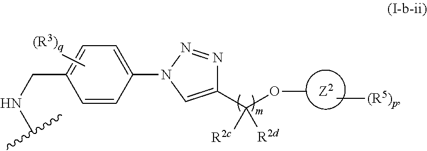

or a pharmaceutically acceptable salt thereof, wherein Ring Z² is cycloalkyl, heterocyclyl, aryl or heteroaryl; each of R^{2c} and R^{2d} is independently hydrogen, alkyl, or heteroalkyl, or R^{2c} and R^{2d} and taken together to form an oxo group; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; each of p and q is independently 0, 1, 2, 3, 4, 5, or 6; and “ [symbol] ” refers to a connection to an attachment group or a polymer described herein.

In some embodiments, the compound of Formula (I) is a compound of Formula (I-c):

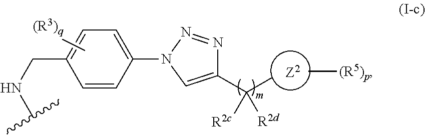

or a pharmaceutically acceptable salt thereof, wherein Ring Z² is cycloalkyl, heterocyclyl, aryl or heteroaryl; each of R^{2c} and R^{2d} is independently hydrogen, alkyl, or heteroalkyl, or each of R^{2c} and R^{2d} is taken together to form an oxo group; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; m is 1, 2, 3, 4, 5, or 6; each of p and q is independently 0, 1, 2, 3, 4, 5, or 6; and “ [symbol] ” refers to a connection to an attachment group or a polymer described herein.

In some embodiments, the compound of Formula (I) is a compound of Formula (I-d):

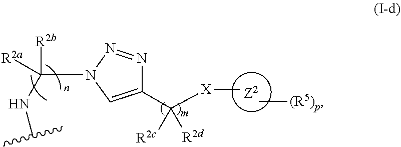

or a pharmaceutically acceptable salt thereof, wherein Ring Z² is cycloalkyl, heterocyclyl, aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I) is a compound of Formula (I-e):

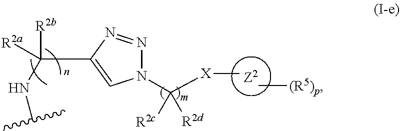

or a pharmaceutically acceptable salt thereof, wherein Ring Z² is cycloalkyl, heterocyclyl, aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I) is a compound of Formula (I-f):

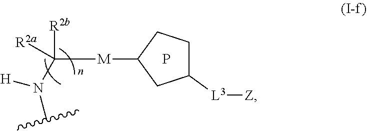

or a pharmaceutically acceptable salt thereof, wherein M is alkyl optionally substituted with one or more R³; Ring P is heteroaryl optionally substituted with one or more R⁴; L³ is alkyl or heteroalkyl optionally substituted with one or more R²; Z is alkyl, heteroalkyl, cycloalkyl, heterocyclyl, aryl, or heteroaryl, each of which is optionally substituted with one or more R⁵; each of R^{2a} and R^{2b} is independently hydrogen, alkyl, or heteroalkyl, or R^{2a} and R^{2b} is taken together to form an oxo group; each R², R³, R⁴, and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (I) is a compound of Formula (II):

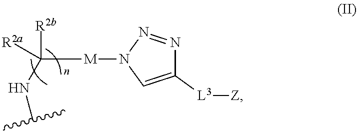

or a pharmaceutically acceptable salt thereof, wherein M is a bond, alkyl or aryl, wherein alkyl and aryl is optionally substituted with one or more R³; L³ is alkyl or heteroalkyl optionally substituted with one or more R²; Z is hydrogen, alkyl, heteroalkyl, cycloalkyl, heterocyclyl, aryl, heteroaryl or —OR^{A}, wherein alkyl, heteroalkyl, cycloalkyl, heterocyclyl, aryl, and heteroaryl is optionally substituted with one or more R⁵; R^{A} is hydrogen; each of R^{2a} and R^{2b} is independently hydrogen, alkyl, or heteroalkyl, or R^{2a} and R^{2b} is taken together to form an oxo group; each R², R³, and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (II) is a compound of Formula (II-a):

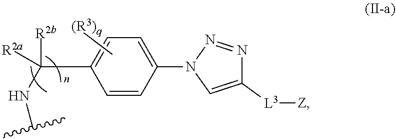

or a pharmaceutically acceptable salt thereof, wherein L³ is alkyl or heteroalkyl, each of which is optionally substituted with one or more R²; Z is hydrogen, alkyl, heteroalkyl, or —OR^{A}, wherein alkyl and heteroalkyl are optionally substituted with one or more R⁵; each of R^{2a} and R^{2b} is independently hydrogen, alkyl, or heteroalkyl, or R^{2a} and R^{2b} is taken together to form an oxo group; each R², R³, and R⁵ is independently alkyl, heteroalkyl, halogen, oxo, —OR^{A1}, —C(O)OR^{A1}, or —C(O)R^{B1}; R^{A} is hydrogen; each R^{A1} and R^{B1} is independently hydrogen, alkyl, or heteroalkyl; n is independently 1, 2, 3, 4, 5, or 6; and [symbol] refers to a connection to an attachment group or a polymer described herein.

In some embodiments, the compound of Formula (I) is a compound of Formula (III):

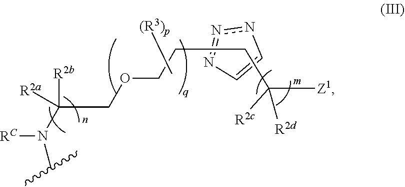

or a pharmaceutically acceptable salt thereof, wherein Z¹ is alkyl, alkenyl, alkynyl, heteroalkyl, cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl; or R^{2a} and R^{2b} or R^{2c} and R^{2d} are taken together to form an oxo group; R^{C} is hydrogen, alkyl, alkenyl, alkynyl, or heteroalkyl, wherein each of alkyl, alkenyl, alkynyl, or heteroalkyl is optionally substituted with 1-6 R⁶; each of R³, R⁵, and R⁶ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; q is an integer from 0 to 25; and “ [symbol] ” refers to a connection to an attachment group or a polymer described herein.

In some embodiments, the compound of Formula (III) is a compound of Formula (III-a):

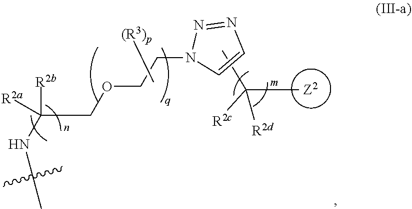

or a pharmaceutically acceptable salt thereof, wherein Ring Z² is cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; o and p are each independently 0, 1, 2, 3, 4, or 5; q is an integer from 0 to 25; and “ [symbol] ” refers to a connection to an attachment group or a polymer described herein.

In some embodiments, the compound of Formula (III-a) is a compound of Formula (III-b):

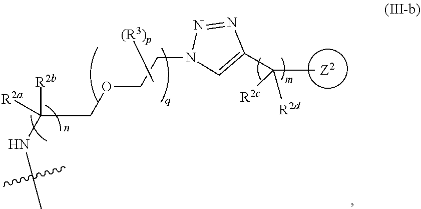

or a pharmaceutically acceptable salt thereof, wherein Ring Z² is cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; o and p are each independently 0, 1, 2, 3, 4, or 5; q is an integer from 0 to 25; and “ [symbol] ” refers to a connection to an attachment group or a polymer described herein.

In some embodiments, the compound of Formula (III-a) is a compound of Formula (III-c):

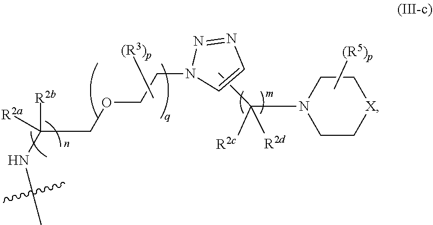

or a pharmaceutically acceptable salt thereof, wherein X is C(R′)(R″), N(R′), or S(O)ₓ; each of R′ and R″ is independently hydrogen, alkyl, halogen, or cycloalkyl; each of R^{2a}, R^{2b}, R^{2c}, and R^{2d} is independently hydrogen, alkyl, heteroalkyl, or halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; p is 0, 1, 2, 3, 4, or 5; q is an integer from 0 to 25; x is 0, 1, or 2; and “ [symbol] ” refers to a connection to an attachment group or a polymer described herein.

In some embodiments, the compound of Formula (III-c) is a compound of Formula (III-d):

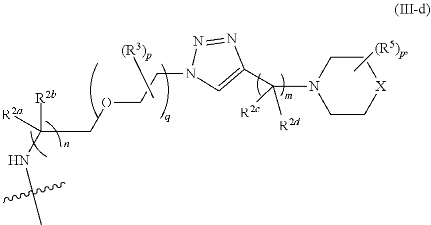

or a pharmaceutically acceptable salt thereof, wherein X is C(R′)(R″), N(R′), or S(O)ₓ; each of R′ and R″ is independently hydrogen, alkyl, halogen, or cycloalkyl; each of R^{2a}, R^{2b}, R^{2c}, and R^{2d} is independently hydrogen, alkyl, heteroalkyl, or halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; p is 0, 1, 2, 3, 4, or 5; q is an integer from 0 to 25; x is 0, 1, or 2; and “ [symbol] ” refers to a connection to an attachment group or a polymer described herein.

In some embodiments, the compound is a compound of Formula (I). In some embodiments, L² is a bond and P and L³ are independently absent.

In some embodiments, the compound is a compound of Formula (I-a). In some embodiments of Formula (II-a), L² is a bond, P is heteroaryl, L³ is a bond, and Z is hydrogen. In some embodiments, P is heteroaryl, L³ is heteroalkyl, and Z is alkyl. In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound is a compound of Formula (I-b). In some embodiments, P is absent, L¹ is —NHCH₂, L² is a bond, M is aryl (e.g., phenyl), L³ is —CH₂O, and Z is heterocyclyl (e.g., a nitrogen-containing heterocyclyl, e.g., thiomorpholinyl-1,1-dioxide). In some embodiments, the compound of Formula (I-b) is Compound 116.

In some embodiments of Formula (I-b), P is absent, L¹ is —NHCH₂, L² is a bond, M is absent, L³ is a bond, and Z is heterocyclyl (e.g., an oxygen-containing heterocyclyl, e.g., tetrahydropyranyl, tetrahydrofuranyl, oxetanyl, or oxiranyl). In some embodiments, the compound of Formula (I-b) is Compound 105.

In some embodiments, the compound is a compound of Formula (I-b-i). In some embodiments of Formula (I-b-i), each of R^{2a} and R^{2b} is independently hydrogen or CH₃, each of R^{2c} and R^{2d} is independently hydrogen, m is 1 or 2, n is 1, X is O, p is O, M² is phenyl optionally substituted with one or more R³, R³ is —CF₃, and Z² is heterocyclyl (e.g., an oxygen-containing heterocyclyl, e.g., tetrahydropyranyl, tetrahydrofuranyl, oxetanyl, or oxiranyl). In some embodiments, the compound of Formula (I-b-i) is Compound 100, Compound 106, Compound 107, Compound 108, Compound 109, or Compound 111.

In some embodiments, the compound is a compound of Formula (I-b-ii). In some embodiments of Formula (I-b-ii), each of R^{2a}, R^{2b}, R^{2c}, and R^{2d} is independently hydrogen, q is 0, p is 0, m is 1, and Z² is heterocyclyl (e.g., an oxygen-containing heterocyclyl, e.g., tetrahydropyranyl). In some embodiments, the compound of Formula (I-b-ii) is Compound 100.

In some embodiments, the compound is a compound of Formula (I-c). In some embodiments of Formula (I-c), each of R^{2c} and R^{2d} is independently hydrogen, m is 1, p is 1, q is 0, R⁵ is —CH₃, and Z is heterocyclyl (e.g., a nitrogen-containing heterocyclyl, e.g., piperazinyl). In some embodiments, the compound of Formula (I-c) is Compound 113.

In some embodiments, the compound is a compound of Formula (I-d). In some embodiments of Formula (I-d), each of R^{2a}, R^{2b}, R^{2c}, and R^{2d} is independently hydrogen, m is 1, n is 3, X is O, p is 0, and Z is heterocyclyl (e.g., an oxygen-containing heterocyclyl, e.g., tetrahydropyranyl, tetrahydrofuranyl, oxetanyl, or oxiranyl). In some embodiments, the compound of Formula (I-d) is Compound 110 or Compound 114.

In some embodiments, the compound is a compound of Formula (I-f). In some embodiments of Formula (I-f), each of R^{2a} and R^{2b} is independently hydrogen, n is 1, M is —CH₂—, P is a nitrogen-containing heteroaryl (e.g., imidazolyl), L³ is —C(O)OCH₂—, and Z is CH₃. In some embodiments, the compound of Formula (I-f) is Compound 115.

In some embodiments, the compound is a compound of Formula (II-a). In some embodiments of Formula (II-a), each of R^{2a} and R^{2b} is independently hydrogen, n is 1, q is 0, L³ is —CH₂(OCH₂CH₂)₂, and Z is —OCH₃. In some embodiments, the compound of Formula (II-a) is Compound 112.

In some embodiments of Formula (II-a), each of R^{2a} and R^{2b} is independently hydrogen, n is 1, L³ is a bond or —CH₂, and Z is hydrogen or —OH. In some embodiments, the compound of Formula (II-a) is Compound 103 or Compound 104.

In some embodiments, the compound is a compound of Formula (III). In some embodiments of Formula (III), each of R^{2a}, R^{2b}, R^{2c}, and R^{2d} is independently hydrogen, m is 1, n is 2, q is 3, p is 0, R^{C} is hydrogen, and Z¹ is heteroalkyl optionally substituted with R⁵ (e.g., —N(CH₃)(CH₂CH₂)S(O)₂CH₃). In some embodiments, the compound of Formula (III) is Compound 120.

In some embodiments, the compound is a compound of Formula (III-b). In some embodiments of Formula (III-b), each of R^{2a}, R^{2b}, R^{2c}, and R^{2d} is independently hydrogen, m is 0, n is 2, q is 3, p is 0, and Z² is aryl (e.g., phenyl) substituted with 1 R⁵ (e.g., —NH₂). In some embodiments, the compound of Formula (III-b) is Compound 102.

In some embodiments, the compound is a compound of Formula (III-b). In some embodiments of Formula (III-b), each of R^{2a}, R^{2b}, R^{2c}, and R^{2d} is independently hydrogen, m is 1, n is 2, q is 3, p is 0, R^{C} is hydrogen, and Z² is heterocyclyl (e.g., an nitrogen-containing heterocyclyl, e.g., a nitrogen-containing spiro heterocyclyl, e.t., 2-oxa-7-azaspiro[3.5]nonanyl). In some embodiments, the compound of Formula (III-b) is Compound 121.

In some embodiments, the compound is a compound of Formula (III-d). In some embodiments of Formula (III-d), each of R^{2a}, R^{2b}, R^{2c}, and R^{2d} is independently hydrogen, m is 1, n is 2, q is 1, 2, 3, or 4, p is 0, and X is S(O)₂. In some embodiments of Formula (III-d), each of R^{2a} and R^{2b} is independently hydrogen, m is 1, n is 2, q is 1, 2, 3, or 4, p is 0, and X is S(O)₂. In some embodiments, the compound of Formula (III-d) is Compound 101, Compound 117, Compound 118, or Compound 119.

In some embodiments, the compound is a compound of Formula (I-b), (I-d), or (I-e). In some embodiments, the compound is a compound of Formula (I-b), (I-d), or (II). In some embodiments, the compound is a compound of Formula (I-b), (I-d), or (I-f). In some embodiments, the compound is a compound of Formula (I-b), (I-d), or (III).

In some embodiments, the compound of Formula (I) is not a compound disclosed in WO2012/112982, WO2012/167223, WO2014/153126, WO2016/019391, WO 2017/075630, US2012-0213708, US 2016-0030359 or US 2016-0030360.

In some embodiments, an implantable element comprises a compound shown in Table 1, or a pharmaceutically acceptable salt thereof.

TABLE 1
Exemplary compounds

| Compound No. | Structure |
|---|---|
| 100 | 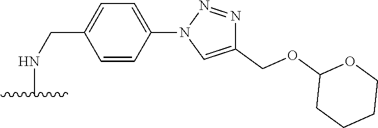 [SMILES: *NCc1ccc(-n2cc(COC3CCCCO3)nn2)cc1] |
| 101 | 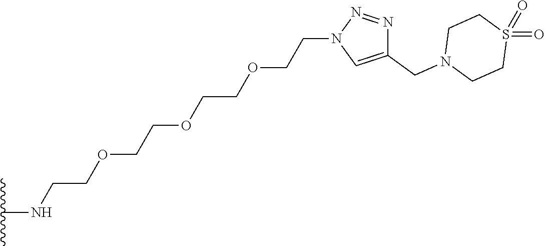 [SMILES: *NCCOCCOCCOCCn1cc(CN2CCS(=O)(=O)CC2)nn1] |
| 102 | 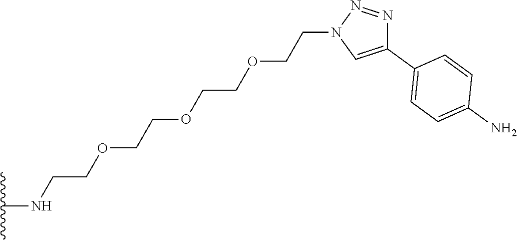 [SMILES: *NCCOCCOCCOCCn1cc(-c2ccc(N)cc2)nn1] |
| 103 | 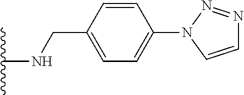 [SMILES: *NCc1ccc(-n2ccnn2)cc1] |
| 104 | 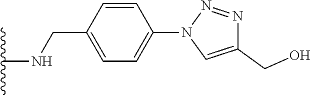 [SMILES: *NCc1ccc(-n2cc(CO)nn2)cc1] |
| 105 | 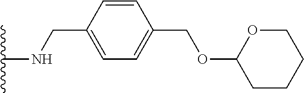 [SMILES: *NCc1ccc(COC2CCCCO2)cc1] |
| 106 | 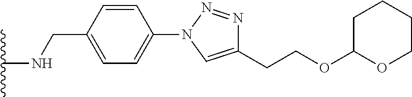 [SMILES: *NCc1ccc(-n2cc(CCOC3CCCCO3)nn2)cc1] |
| 107 | 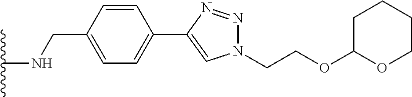 [SMILES: *NCc1ccc(-c2cn(CCOC3CCCCO3)nn2)cc1] |
| 108 | 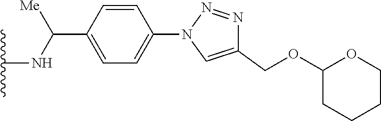 [SMILES: *NC(C)c1ccc(-n2cc(COC3CCCCO3)nn2)cc1] |
| 109 | 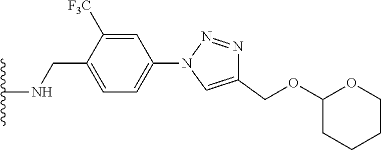 [SMILES: *NCc1ccc(-n2cc(COC3CCCCO3)nn2)cc1C(F)(F)F] |
| 110 | 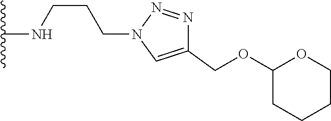 [SMILES: *NCCCn1cc(COC2CCCCO2)nn1] |
| 111 | 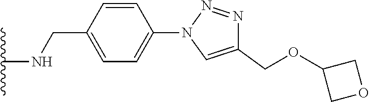 [SMILES: *NCc1ccc(-n2cc(COC3COC3)nn2)cc1] |
| 112 | 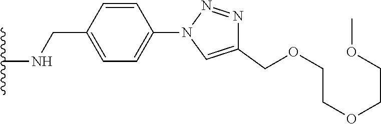 [SMILES: *NCc1ccc(-n2cc(COCCOCCOC)nn2)cc1] |
| 113 | 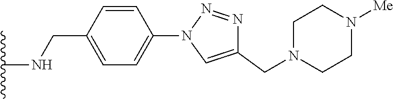 [SMILES: *NCc1ccc(-n2cc(CN3CCN(C)CC3)nn2)cc1] |
| 114 | 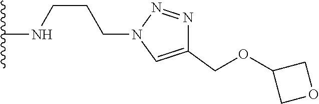 [SMILES: *NCCCn1cc(COC2COC2)nn1] |
| 115 | 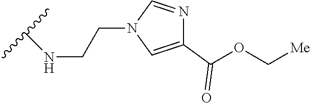 [SMILES: *NCCn1cnc(C(=O)OCC)c1] |
| 116 | 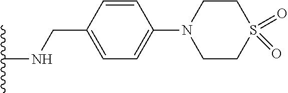 [SMILES: *NCc1ccc(N2CCS(=O)(=O)CC2)cc1] |
| 117 | 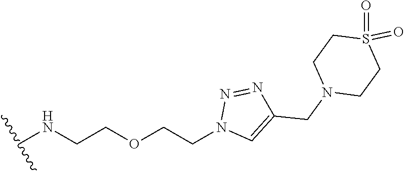 [SMILES: *NCCOCCn1cc(CN2CCS(=O)(=O)CC2)nn1] |
| 118 | 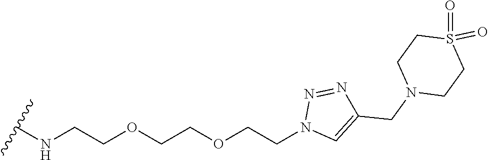 [SMILES: *NCCOCCOCCn1cc(CN2CCS(=O)(=O)CC2)nn1] |
| 119 | 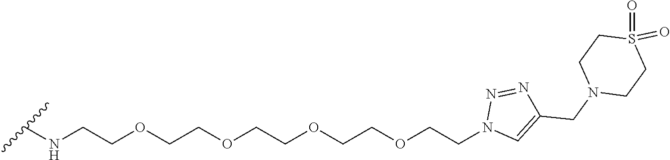 [SMILES: *NCCOCCOCCOCCOCCn1cc(CN2CCS(=O)(=O)CC2)nn1] |
| 120 | 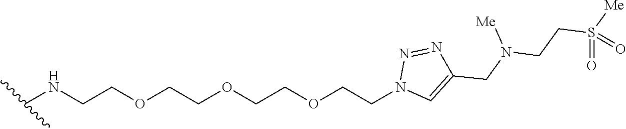 [SMILES: *NCCOCCOCCOCCn1cc(CN(C)CCS(C)(=O)=O)nn1] |
| 121 | 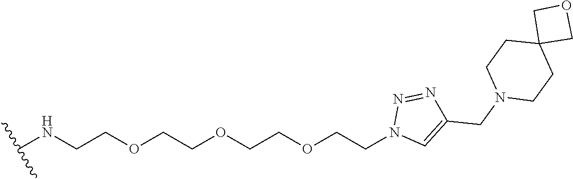 [SMILES: *NCCOCCOCCOCCn1cc(CN2CCC3(CC2)COC3)nn1] |

In some embodiments, the compound of Formula (I) (e.g., Formulas (I-a), (I-b), (I-c), (I-d), (I-e), (I-f), (II), (II-a), (III), (III-a), (III-b), (III-c), or (III-d)), or a pharmaceutically acceptable salt thereof is selected from:

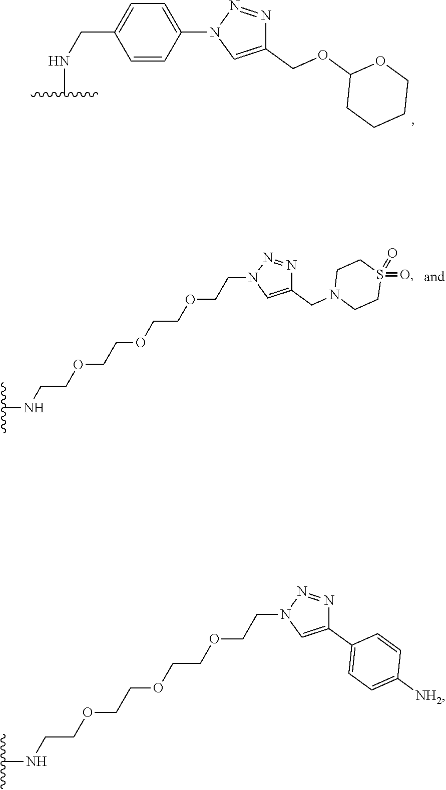 [SMILES: *NCc1ccc(-n2cc(COC3CCCCO3)nn2)cc1 ; *NCCOCCOCCOCCn1cc(CN2CCS(=O)(=O)CC2)nn1 ; *NCCOCCOCCOCCn1cc(-c2ccc(N)cc2)nn1]

or a salt thereof.

In some embodiments, the compound of Formula (I) described herein is selected from:

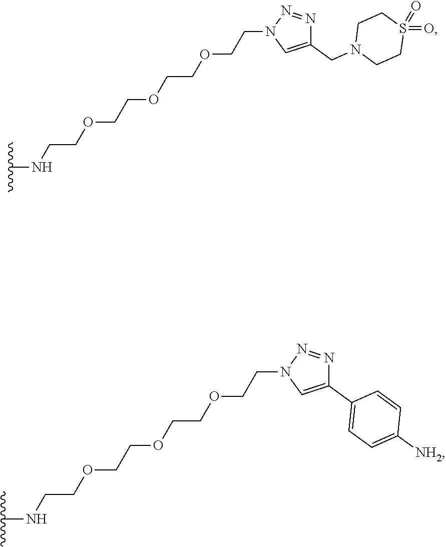 [SMILES: *NCCOCCOCCOCCn1cc(CN2CCS(=O)(=O)CC2)nn1 ; *NCCOCCOCCOCCn1cc(-c2ccc(N)cc2)nn1]

or a pharmaceutically acceptable salt of either compound.

### Features of Chemically Modified Implantable Elements

An implantable element may be coated with a compound of Formula (I) or a pharmaceutically acceptable salt thereof, or a material comprising a compound of Formula (I) or a pharmaceutically acceptable salt thereof. In an embodiment, the compound of Formula (I) is disposed on a surface, e.g., an inner or outer surface, of the implantable element. In some embodiments, the compound of Formula (I) is disposed on a surface, e.g., an inner or outer surface, of an enclosing component associated with an implantable element. In an embodiment, the compound of Formula (I) is distributed evenly across a surface. In an embodiment, the compound of Formula (I) is distributed unevenly across a surface.

In some embodiments, an implantable element (e.g., or an enclosing component thereof) is coated (e.g., covered, partially or in full), with a compound of Formula (I) or a material comprising Formula (I) or a pharmaceutically acceptable salt thereof. In some embodiments, a implantable element (e.g., or an enclosing component thereof) is coated with a single layer of a compound of Formula (I). In some embodiments, a device is coated with multiple layers of a compound of Formula (I), e.g., at least 2 layers, 3 layers, 4 layers, 5 layers, 10 layers, 20 layers, 50 layers or more.

In an embodiment, a first portion of the surface of the implantable element comprises a compound of Formula (I) that modulates, e.g., downregulates or upregulates, a biological function and a second portion of the implantable element lacks the compound, or has substantially lower density of the compound.

In an embodiment a first portion of the surface of the implantable element comprises a compound of Formula (I) that modulates, e.g., down regulates, an immune response and a second portion of the surface comprises a second compound of Formula (I), e.g., that upregulates the immune response, second portion of the implantable element lacks the compound, or has substantially lower density of the compound.

In some embodiments, an implantable element is coated or chemically derivatized in a symmetrical manner with a compound of Formula (I), or a material comprising Formula (I), or a pharmaceutically acceptable salt thereof. In some embodiments, an implantable element is coated or chemically derivatized in an asymmetrical manner with a compound of Formula (I), or a material comprising Formula (I), or a pharmaceutically acceptable salt thereof. For example, an exemplary implantable element may be partially coated (e.g., at least about 5%, 10%, 15%, 20%, 25%, 30%, 35%, 40%, 45%, 50%, 55%, 60%, 65%, 70%, 75%, 80%, 85%, 90%, 95%, 99%, or 99.9% coated) with a compound of Formula (I) or a material comprising a compound of Formula (I) or a pharmaceutically acceptable salt thereof.

Exemplary implantable elements coated or chemically derivatized with a compound of Formula (I), or a material comprising Formula (I), or a pharmaceutically acceptable salt thereof may be prepared using any method known in the art, such as through self-assembly (e.g., via block copolymers, adsorption (e.g., competitive adsorption), phase separation, microfabrication, or masking).

In some embodiments, the implantable element comprises a surface exhibiting two or more distinct physicochemical properties (e.g., 3, 4, 5, 6, 7, 8, 9, 10, or more distinct physicochemical properties).

In some embodiments, the coating or chemical derivatization of the surface of an exemplary implantable element with a compound of Formula (I), a material comprising a compound of Formula (I), or a pharmaceutically acceptable salt thereof is described as the average number of attached compounds per given area, e.g., as a density. For example, the density of the coating or chemical derivatization of an exemplary implantable element may be 0.01, 0.1, 0.5, 1, 5, 10, 15, 20, 50, 75, 100, 200, 400, 500, 750, 1,000, 2,500, or 5,000 compounds per square μm or square mm, e.g., on the surface or interior of said implantable element.

An implantable element comprising a compound of Formula (I) or a pharmaceutically acceptable salt thereof may have a reduced immune response (e.g., a marker of an immune response) compared to an implantable element that does not comprise a compound of Formula (I) or a pharmaceutically acceptable salt thereof. A marker of immune response is one or more of: cathepsin level or the level of a marker of immune response, e.g., TNF-α, IL-13, IL-6, G-CSF, GM-CSF, IL-4, CCL2, or CCL4, as measured, e.g., by ELISA. In some embodiments, an implantable element comprising a compound of Formula (I) or a pharmaceutically acceptable salt thereof has about a 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or about 100% reduced immune response (e.g., a marker of an immune response) compared to an implantable element that does not comprise a compound of Formula (I) or a pharmaceutically acceptable salt thereof. In some embodiments, the reduced immune response (e.g., a marker of an immune response) is measured after about 30 minutes, about 1 hour, about 6 hours, about 12 hours, about 1 day, about 2 days, about 3 days, about 4 days, about 1 week, about 2 weeks, about 1 month, about 2 months, about 3 months, about 6 months, or longer. In some embodiments, an implantable element comprising a compound of Formula (I) is coated by the compound of Formula (I) or encapsulated a compound of Formula (I).

An implantable element comprising a compound of Formula (I) or a pharmaceutically acceptable salt thereof may have an increased immune response (e.g., a marker of an immune response) compared to an implantable element that does not comprise a compound of Formula (I) or a pharmaceutically acceptable salt thereof. A marker of immune response is one or more of: cathepsin activity, or the level of a marker of immune response, e.g., TNF-α, IL-13, IL-6, G-CSF, GM-CSF, IL-4, CCL2, or CCL4, as measured, e.g., by ELISA. In some embodiments, a device comprising a compound of Formula (I) or a pharmaceutically acceptable salt thereof has about a 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or about 100%, or about 1000% increased immune response (e.g., a marker of an immune response) compared to an implantable element that does not comprise a compound of Formula (I) or a pharmaceutically acceptable salt thereof. In some embodiments, the increased immune response (e.g., a marker of an immune response) is measured after about 30 minutes, about 1 hour, about 6 hours, about 12 hours, about 1 day, about 2 days, about 3 days, about 4 days, about 1 week, about 2 weeks, about 1 month, about 2 months, about 3 months, about 6 months, or longer. In some embodiments, an implantable element comprising a compound of Formula (I) is coated by the compound of Formula (I) or encapsulated a compound of Formula (I).

An implantable element may have a smooth surface, or may comprise a protuberance, depression, well, slit, or hole, or any combination thereof. Said protuberance, depression, well, slit or hole may be any size, e.g., from 10 μm to about 1 nm, about 5 μm to about 1 nm, about 2.5 μm to about 1 nm, 1 μm to about 1 nm, 500 nm to about 1 nm, or about 100 nm to about 1 nm. The smooth surface or protuberance, depression, well, slit, or hole, or any combination thereof, may be coated or chemically derivatized with a compound of Formula (I), a material comprising a compound of Formula (I), or a pharmaceutically acceptable salt thereof.

An implantable element may take any suitable shape, such as a sphere, spheroid, ellipsoid, disk, cylinder, torus, cube, stadiumoid, cone, pyramid, triangle, rectangle, square, or rod, or may comprise a curved or flat section. Any shaped, curved, or flat implantable element may be coated or chemically derivatized with a compound of Formula (I), a material comprising a compound of Formula (I), or a pharmaceutically acceptable salt thereof.

### Methods of Treatment

Described herein are methods for preventing or treating a disease, disorder, or condition in a subject through administration or implantation of MSFC, e.g., encapsulated by a material or device described herein. In some embodiments, the methods described herein directly or indirectly reduce or alleviate at least one symptom of a disease, disorder, or condition. In some embodiments, the methods described herein prevent or slow the onset of a disease, disorder, or condition. In some embodiments, the subject is a human.

In some embodiments, the disease, disorder, or condition affects a system of the body, e.g. the nervous system (e.g., peripheral nervous system (PNS) or central nervous system (CNS)), vascular system, skeletal system, respiratory system, endocrine system, lymph system, reproductive system, or gastrointestinal tract. In some embodiments, the disease, disorder, or condition affects a part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis.

In some embodiments, the disease, disorder or condition is a neurodegenerative disease, diabetes, a heart disease, an autoimmune disease, a cancer, a liver disease, a lysosomal storage disease, a blood clotting disorder or a coagulation disorder, an orthopedic conditions, an amino acid metabolism disorder.

In some embodiments, the disease, disorder or condition is a neurodegenerative disease. Exemplary neurodegenerative diseases include Alzheimer's disease, Huntington's disease, Parkinson's disease (PD) amyotrophic lateral sclerosis (ALS), multiple sclerosis (MS) and cerebral palsy (CP), dentatorubro-pallidoluysian atrophy (DRPLA), neuronal intranuclear hyaline inclusion disease (NIHID), dementia with Lewy bodies, Down's syndrome, Hallervorden-Spatz disease, prion diseases, argyrophilic grain dementia, cortocobasal degeneration, dementia pugilistica, diffuse neurofibrillary tangles, Gerstmann-Straussler-Scheinker disease, Jakob-Creutzfeldt disease, Niemann-Pick disease type 3, progressive supranuclear palsy, subacute sclerosing panencephalitis, spinocerebellar ataxias, Pick's disease, and dentatorubral-pallidoluysian atrophy.

In some embodiments, the disease, disorder, or condition is an autoimmune disease, e.g., scleroderma, multiple sclerosis, lupus, or allergies.

In some embodiments, the disease is a liver disease, e.g., hepatitis B, hepatitis C, cirrhosis, NASH.

In some embodiments, the disease, disorder, or condition is cancer. Exemplary cancers include leukemia, lymphoma, melanoma, lung cancer, brain cancer (e.g., glioblastoma), sarcoma, pancreatic cancer, renal cancer, liver cancer, testicular cancer, prostate cancer, or uterine cancer.

In some embodiments, the disease, disorder, or condition is an orthopedic condition. Exemplary orthopedic conditions include osteoporosis, osteonecrosis, Paget's disease, or a fracture.

In some embodiments, the disease, disorder or condition is a lysosomal storage disease. Exemplary lysosomal storage diseases include Gaucher disease (e.g., Type I, Type II, Type III), Tay-Sachs disease, Fabry disease, Farber disease, Hurler syndrome (also known as mucopolysaccharidosis type I (MPS I)), Hunter syndrome, lysosomal acid lipase deficiency, Niemann-Pick disease, Salla disease, Sanfilippo syndrome (also known as mucopolysaccharidosis type IIIA (MPS3A)), multiple sulfatase deficiency, Maroteaux-Lamy syndrome, metachromatic leukodystrophy, Krabbe disease, Scheie syndrome, Hurler-Scheie syndrome, Sly syndrome, hyaluronidase deficiency, Pompe disease, Danon disease, gangliosidosis, or Morquio syndrome.

In some embodiments, the disease, disorder, or condition is a blood clotting disorder or a coagulation disorder. Exemplary blood clotting disorders or coagulation disorders include hemophilia (e.g., hemophilia A or hemophilia B), Von Willebrand disease, thrombocytopenia, uremia, Bernard-Soulier syndrome, Factor XII deficiency, vitamin K deficiency, or congenital afibrinogenimia.

In some embodiments, the disease, disorder, or condition is an amino acid metabolism disorder, e.g., phenylketonuria, tyrosinemia (e.g., Type 1 or Type 2), alkaptonuria, homocystinuria, hyperhomocysteinemia, maple syrup urine disease.

In some embodiments, the disease, disorder, or condition is a fatty acid metabolism disorder, e.g., hyperlipidemia, hypercholesterolemia, galactosemia.

In some embodiments, the disease, disorder, or condition is a purine or pyrimidine metabolism disorder, e.g., Lesch-Nyhan syndrome.

In some embodiments, the disease, disorder, or condition is not diabetes (e.g., Type I or Type II diabetes).

The present disclosure further comprises methods for identifying a subject having or suspected of having a disease, disorder, or condition described herein, and upon such identification, administering to the subject implantable element comprising an MSFC (e.g., an MSC), e.g., optionally encapsulated by an enclosing component, and optionally modified with a compound of Formula (I) as described herein, or a composition thereof. In an embodiment, the subject is a human.

### Pharmaceutical Compositions, Kits, and Administration

The present disclosure further comprises implantable elements comprising MSFCs (e.g., MSCs), as well as pharmaceutical compositions comprising the same, and kits thereof.

In some embodiments, a pharmaceutical composition comprises MSFCs and a pharmaceutically acceptable excipient. In some embodiments, a pharmaceutical composition comprises engineered MSFCs and a pharmaceutically acceptable excipient. In some embodiments, MSFCs are provided in an effective amount in the pharmaceutical composition. In some embodiments, the effective amount is a therapeutically effective amount. In some embodiments, the effective amount is a prophylactically effective amount.

Pharmaceutical compositions described herein can be prepared by any method known in the art of pharmacology. In general, such preparatory methods include the steps of bringing the MSFCs (e.g., MSCs, i.e., “the active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the disclosure will vary, depending upon the identity, size, and/or condition of the subject treated and further depending upon the route by which the composition is to be administered. By way of example, the composition may comprise between 0.1% and 100% (w/w) active ingredient.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the disclosure are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The MSFCs, implantable elements, and compositions thereof, may be administered orally, parenterally (including subcutaneous, intramuscular, and intradermal), topically, rectally, nasally, intratumorally, intrathecally, buccally, vaginally or via an implanted reservoir. In some embodiments, provided compounds or compositions are administrable subcutaneously or by implant.

In some embodiments, the MSFCs, implantable elements, and compositions thereof, may be administered or implanted in or on a certain region of the body, such as a mucosal surface or a body cavity. Exemplary sites of administration or implantation include the peritoneal cavity (e.g., lesser sac), adipose tissue, heart, eye, muscle, spleen, lymph node, esophagus, nose, sinus, teeth, gums, tongue, mouth, throat, small intestine, large intestine, thyroid, bone (e.g., hip or a joint), breast, cartilage, vagina, uterus, fallopian tube, ovary, penis, testicles, blood vessel, liver, kidney, central nervous system (e.g., brain, spinal cord, nerve), or ear (e.g., cochlea).

In some embodiments, the MSFCs, implantable elements, and compositions thereof, are administered or implanted at a site other than the central nervous system, e.g., the brain, spinal cord, nerve. In some embodiments, the MSFCs, implantable elements, and compositions thereof, are administered or implanted at a site other than the eye (e.g., retina).

Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For ophthalmic use, provided pharmaceutically acceptable compositions may be formulated as micronized suspensions or in an ointment such as petrolatum.

In order to prolong the effect of the active ingredient, it may be desirable to slow the absorption of the drug from subcutaneous or intramuscular injection.

In some embodiments, MSFCs are disposed on a microcarrier (e.g., a bead, e.g., a polystyrene bead).

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

The MSFCs, implantable elements, and the compositions thereof may be formulated in dosage unit form, e.g., single unit dosage form, for ease of administration and uniformity of dosage. It will be understood, however, that the total dosage and usage regimens of the compositions of the present disclosure will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

It will be appreciated that the composition, as described herein, can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The composition can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and a pain-relieving agent. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. Also encompassed by the disclosure are kits (e.g., pharmaceutical packs). The inventive kits may be useful for preventing and/or treating any of the diseases, disorders or conditions described herein. The kits provided may comprise an inventive pharmaceutical composition or device and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or device. In some embodiments, the inventive pharmaceutical composition or device provided in the container and the second container are combined to form one unit dosage form.

### EXAMPLES

In order that the disclosure described herein may be more fully understood, the following examples are set forth. The examples described in this application are offered to illustrate the MSFCs (e.g., MSCs), implantable elements, chemical modifications, and compositions and methods provided herein and are not to be construed in any way as limiting their scope.

### Example 1: Culturing MSFCs

Human MSC cells may be cultured according to any method known in the art, such as according to the following protocols used for human MSCs marketed by RoosterBio, Inc. (Frederick, Md.) and ATCC (Manassas, Va.).

Cryopreserved human bone marrow-derived MSCs obtained from RoosterBio (MSC-003) were cultured using the Rooster Basal™-MSC medium in the RoosterNourish™ MSC (KT-001) in a 75 cm² culture flask at 37° C. until the desired confluency was reached, with media changed every 2 or 3 days When the culture was 80% confluent, the cells were aspirated the following day to remove culture medium, and the cell layer was briefly rinsed with 0.05% (w/v) trypsin/0.53 mM EDTA solution (“TrypsinEDTA”) to remove all traces of serum that contains a trypsin inhibitor. 2-3 mL Trypsin/EDTA solution was added to the flask, and the cells were observed under an inverted microscope until the cell layer was dispersed, usually between 5-15 minutes. To avoid clumping, cells were handled with care during the dispersion period. If the cells did not detach, the flasks were placed at 37° C. to facilitate dispersal. Once the cells had dispersed, 6-8 mL of the same Rooster Bio culture media was added and the cells were aspirated by gentle pipetting. The cell suspension was transferred to a centrifuge tube and spun down at approximately 200×g for 5 to 10 minutes to remove TrypsinEDTA. The supernatant was discarded, and the cells were resuspended in fresh culture media (same as above). Appropriate aliquots of cell suspension were added to new culture vessels, which were incubated at 37° C. using the same culture conditions as above.

Human MSC cells obtained from ATCC (Bone Marrow-Derived Mesenchymal Stem Cells; Normal, Human (ATCC PCS-500-012™)) were cultured as described above except the culture medium used was Mesenchymal Stem Cell Basal Medium for Adipose, Umbilical and Bone Marrow-derived MSCs (ATCC PCS-500030™) supplemented with the Mesenchymal Stem Cell Growth Kit for Bone Marrow-derived MSCs (ATCC PCS-500-041™).

### Example 2: Preparation of MSFC Clusters

Spheroid clusters of human bone marrow-derived MSCs obtained from RoosterBio (MSC-003) were prepared using AggreWell™ spheroid plates (STEMCELL Technologies) and the protocol outlined herein. On Day 1, rinsing solution (4 mL) was added to each plate, and the plates were spun down for 5 minutes at 3,000 RPM in a large centrifuge. The rinsing solution was removed by pipet, and 4 mL of the same RoosterBio media used in Example 1 was added. The cells were seeded into the plates at the desired cell density and pipetted immediately to prevent aggregation, with the general rule of thumb that 3.9 million cells per well will generate 150 μm diameter clusters. The plate was spun down for 3 minutes at 800 RPM, and the plate was placed into an incubator at 37° C. overnight.

On Day 2, the plate was removed from incubation. Using wide bore pipet tips, the cells were gently pipetted to dislodge the spheroid clusters. The clusters were filtered through a 40 μm or 80 μm cell strainer to remove extraneous detached single cells and then spun down in a centrifuge for 2×1 minute. The clusters were resuspended gently using wide bore pipet tips and were gently stirred to distribute them throughout the medium or another material (e.g., alginate).

Alternatively, human MSC spheroids may be prepared using the following protocol. On Day 1, AggreWell™ plates are removed from the packaging in a sterile tissue culture hood. Add 2 mL of Aggrewell™ Rinsing solution to each well. Centrifuge the plate at 2,000 g for 5 minutes to remove air bubbles. Remove AggreWell™ Rinsing Solution from the wells and rinse each well with 2 mL of the desired culture media. Add 2 million human MSC cells in 3.9 mL of the desired culture medium for each well. Centrifuge the plate at 100 g for 3 minutes. Incubate the cells at 37° C. for 48 hours. On Day 3, the same protocol described above is used to dislodge the spheroid clusters.

### Example 3: Synthesis of Exemplary Compounds for Preparation of Chemically Modified Implantable Elements

### General Protocols

The procedures below describe methods of preparing exemplary compounds for preparation of chemically modified implantable elements. The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

### Huisgen Cycloaddition to Afford 1,4-Substituted Triazoles

The copper-catalyzed Huisgen [3+2] cycloaddition was used to prepare triazole-based compounds and compositions, devices, and materials thereof. The scope and typical protocols have been the subject of many reviews (e.g., Meldal, M. and Tornoe, C. W. Chem. Rev. (2008) 108:2952-3015; Hein, J. E. and Fokin, V. V. Chem. Soc. Rev. (2010) 39(4):1302-1315; both of which are incorporated herein by reference).

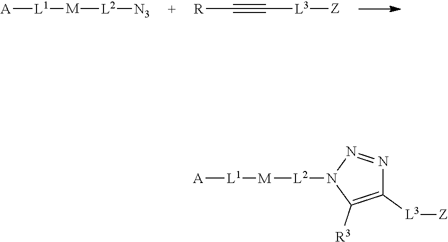

In the example shown above, the azide is the reactive moiety in the fragment containing the connective element A, while the alkyne is the reactive component of the pendant group Z. As depicted below, these functional handles can be exchanged to produce a structurally related triazole product. The preparation of these alternatives is similar, and do not require special considerations.

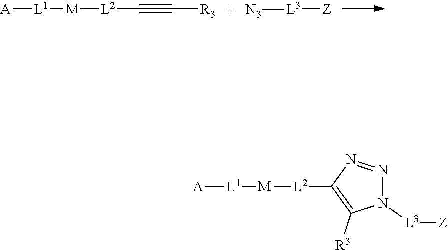

A typical Huisgen cycloaddition procedure starting with an iodide is outlined below. In some instances, iodides are transformed into azides during the course of the reaction for safety.

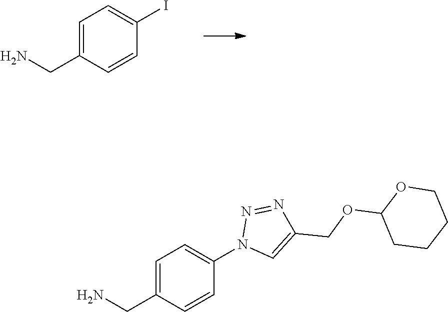 [SMILES: NCc1ccc(I)cc1 ; NCc1ccc(-n2cc(COC3CCCCO3)nn2)cc1]

A solution of sodium azide (1.1 eq), sodium ascorbate, (0.1 eq) trans-N,N′-dimethylcyclohexane-1,2-diamine (0.25 eq), copper (I) iodide in methanol (1.0 M, limiting reagent) was degassed with bubbling nitrogen and treated with the acetylene (1 eq) and the aryl iodide (1.2 eq). This mixture was stirred at room temperature for 5 minutes, then warmed to 55° C. for 16 h. The reaction was then cooled to room temperature, filtered through a funnel, and the filter cake washed with methanol. The combined filtrates were concentrated and purified via flash chromatography on silica gel (120 g silica, gradient of 0 to 40% (3% aqueous ammonium hydroxide, 22% methanol, remainder dichloromethane) in dichloromethane to afford the desired target material.

A typical Huisgen cycloaddition procedure starting with an azide is outlined below.

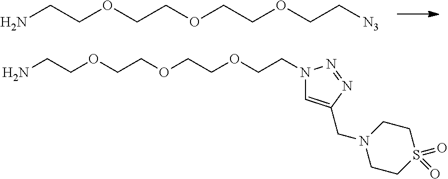

A solution of tris[(1-benzyl-1H-1,2,3-triazol-4-yl)methyl]amine (0.2 eq), triethylamine (0.5 eq), copper (I) iodide (0.06 eq) in methanol (0.4 M, limiting reagent) was treated with the acetylene (1.0 eq) and cooled to 0° C. The reaction was allowed to warm to room temperature over 30 minutes, then heated to 55° C. for 16 h. The reaction was cooled to room temperature, concentrated, and purified with HPLC (C18 column, gradient of 0 to 100% (3% aqueous ammonium hydroxide, 22% methanol remainder dichloromethane) in dichloromethane to afford the desired target material.

### Huisgen Cycloaddition to Afford 1,5-Substituted Triazoles

The Huisgen [3+2] cycloaddition was also performed with ruthenium catalysts to obtain 1,5-disubstituted products preferentially (e.g., as described in Zhang et al, J. Am. Chem. Soc., 2005, 127, 15998-15999; Boren et al, J. Am. Chem. Soc., 2008, 130, 8923-8930, each of which is incorporated herein by reference in its entirety).

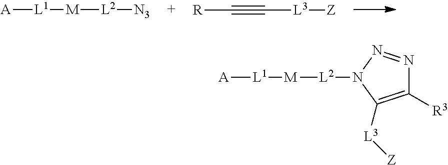

As described previously, the azide and alkyne groups may be exchanged to form similar triazoles as depicted below.

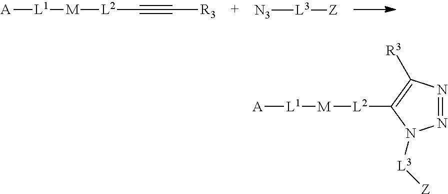

A typical procedure is described as follows: a solution of the alkyne (1 eq) and the azide (1 eq) in dioxane (0.8M) were added dropwise to a solution of pentamethylcyclo-pentadienylbis(triphenylphosphine) ruthenium(II) chloride (0.02 eq) in dioxane (0.16M). The vial was purged with nitrogen, sealed and the mixture heated to 60° C. for 12 h. The resulting mixture was concentrated and purified via flash chromatography on silica gel to afford the requisite compound.

### Experimental Procedure for (4-(4-((4-methylpiperazin-1-yl)methyl)-1H-1,2,3-triazol-1-yl)phenyl)methanamine (3)

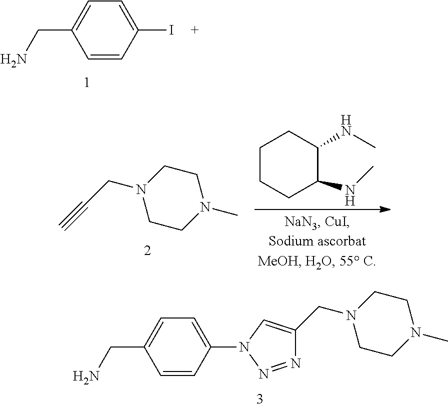

A mixture of (4-iodophenyl)methanamine (1, 843 mg, 3.62 mmol, 1.0 eq), (1S,2S)—N1,N2-dimethylcyclohexane-1,2-diamine (74 μL, 0.47 mmol, 0.13 eq), Sodium ascorbate (72 mg, 0.36 mmol, 0.1 eq), Copper Iodide (69 mg, 0.36 mmol, 0.1 eq), Sodium azide (470 mg, 7.24 mmol, 2.0 eq), and 1-methyl-4-(prop-2-yn-1-yl)piperazine (2, 0.5 g, 3.62 mmol, 1.0 eq) in Methanol (9 mL) and water (1 mL) were purged with nitrogen for 5 minutes and heated to 55° C. for over night. The reaction mixture was cooled to room temperature, concentrated under reduced pressure, and the brownish slurry was extracted with dichloromethane. Celite was added to the combined dichloromethane phases and the solvent was removed under reduced pressure. The crude product was purified over silica gel (80 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 7.5% to afford (4-(4-((4-methylpiperazin-1-yl)methyl)-1H-1,2,3-triazol-1-yl)phenyl)methanamine (3, 0.45 g, 43%). LCMS m/z: [M+H]⁺ Calcd for C₁₅H₂₂N₆ 287.2; Found 287.1.

### Experimental Procedure for N-(4-(4-((4-methylpiperazin-1-yl)methyl)-1H-1,2,3-triazol-1-yl)benzyl)methacrylamide (4)

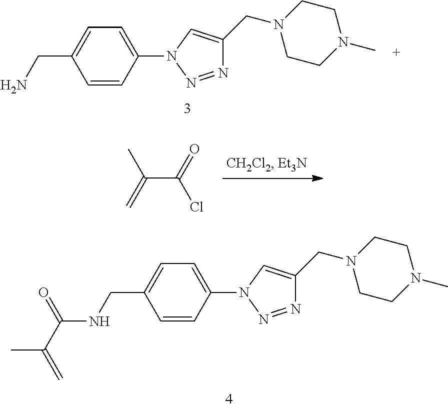

A solution of (4-(4-((4-methylpiperazin-1-yl)methyl)-1H-1,2,3-triazol-1-yl)phenyl)methanamine (3, 1.2 g, 4.19 mmol, 1.0 eq) and triethylamine (0.70 mL, 5.03 mmol, 1.2 eq) in CH₂Cl₂ (50 mL) was cooled to 0° C. with an ice-bath and methacryloyl chloride (0.43 mL, 4.40 mmol, 1.05 eq in 5 mL of CH₂Cl₂) was added. The reaction was stirred for a day while cooled with an ice-bath. 10 grams of Celite were added and the solvent was removed under reduced pressure. The residue was purified by silica gel chromatography (80 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 7.5%. The solvent was removed under reduced pressure and the resulting solid was triturated with diethyl ether, filtered and washed multiple times with diethyl ether to afford N-(4-(4-((4-methylpiperazin-1-yl)methyl)-1H-1,2,3-triazol-1-yl)benzyl)methacrylamide (4, 0.41 g, 28% yield) as a white solid. LCMS m/z: [M+H]⁺ Calcd for C₁₉H₂₆N₆O 355.2; Found 355.2.

### Experimental Procedure for (4-(4-((2-(2-methoxyethoxy)ethoxy)methyl)-1H-1,2,3-triazol-1-yl)phenyl)methanamine (6)

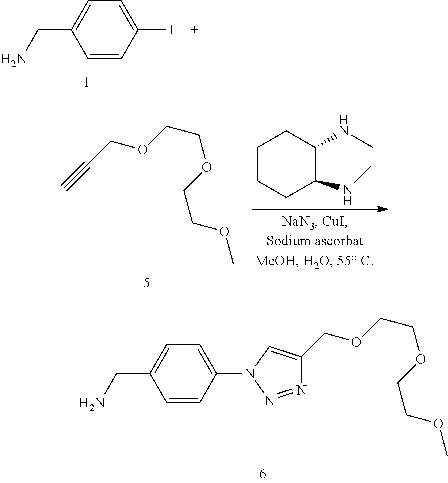

A mixture of (4-iodophenyl)methanamine (1, 2.95 g, 12.64 mmol, 1.0 eq), (1S,2S)—N1,N2-dimethylcyclohexane-1,2-diamine (259 μL, 1.64 mmol, 0.13 eq), Sodium ascorbate (250 mg, 1.26 mmol, 0.1 eq), Copper Iodide (241 mg, 1.26 mmol, 0.1 eq), Sodium azide (1.64 g, 25.29 mmol, 2.0 eq), and 1-methyl-4-(prop-2-yn-1-yl)piperazine (5, 2.0 g, 12.64 mmol, 1.0 eq) in Methanol (40 mL) and water (4 mL) were purged with Nitrogen for 5 minutes and heated to 55° C. overnight. The reaction mixture was cooled to room temperature and concentrated under reduced pressure. The residue was dissolved in dichloromethane, filtered, and concentrated with Celite (10 g). The crude product was purified by silica gel chromatography (220 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 6.25% to afford (4-(4-((2-(2-methoxyethoxy)ethoxy)methyl)-1H-1,2,3-triazol-1-yl)phenyl)methanamine (6, 1.37 g, 35%). LCMS m/z: [M+H]⁺ Calcd for C₁₅H₂₂N₄O₃ 307.2; Found 307.0.

### Experimental Procedure for N-(4-(4-((2-(2-methoxyethoxy)ethoxy)methyl)-1H-1,2,3-triazol-1-yl)benzyl)methacrylamide (7)

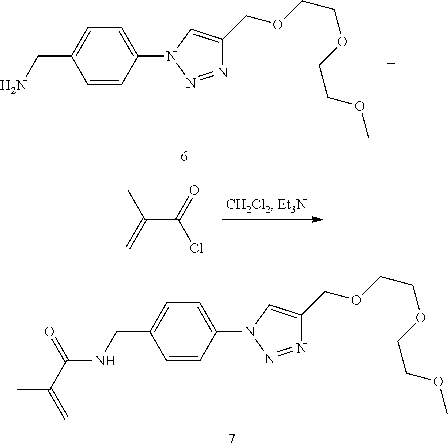

A solution of 4-(4-((2-(2-methoxyethoxy)ethoxy)methyl)-1H-1,2,3-triazol-1-yl)phenyl)methanamine (6, 1.69 g, 5.52 mmol, 1.0 eq) and triethylamine (0.92 mL, 6.62 mmol, 1.2 eq) in CH₂Cl₂ (50 mL) was cooled to 0° C. with an ice-bath and methacryloyl chloride (0.57 mL, 5.79 mmol, 1.05 eq) was added in a dropwise fashion. The reaction was stirred for 4 h at room temperature. 10 grams of Celite were added and the solvent was removed under reduced pressure. The residue was purified by silica gel (80 g) chromatography using dichloromethane/(methanol containing 12% (v/v) aqueous ammonium hydroxide) as mobile phase. The concentration of (methanol containing 12% (v/v) aqueous ammonium hydroxide) was gradually increased from 0% to 1.25% to afford N-(4-(4-((2-(2-methoxyethoxy)ethoxy)methyl)-1H-1,2,3-triazol-1-yl)benzyl)methacrylamide (7, 1.76 g, 85% yield) as a white solid. LCMS m/z: [M+H]⁺ Calcd for C₁₉H₂₆N₄O₄ 375.2; Found 375.0.

### Experimental Procedure for 3-(prop-2-yn-1-yloxy)oxetane (9)

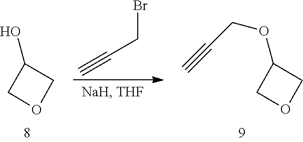

A suspension of sodium hydride (27.0 g, 675 mmol, 60% purity) in THF (200 mL) was cooled with an ice bath. Oexetan-3-ol (8, 25 g, 337 mmol) was added in a dropwise fashion and stirred for 30 minutes at 0° C. 3-Bromoprop1-yne (9, 41.2 mL, 371 mmol, 80% purity) was then added in a dropwise fashion. The mixture was stirred over night while allowed to warm to room temperature. The mixture was filtered over Celite, washed with THF, and concentrated with Celite under reduced pressure. The crude product was purified over silica gel (220 g) and eluted with Hexanes/EtOAc. The concentration of EtOAc in the mobile phase was increased from 0 to 25% to afford a yellow oil of (9, 18.25 g 48%).

### Experimental Procedure for 3-(4-((oxetan-3-yloxy)methyl)-1H-1,2,3-triazol-1-yl)propan-1-amine (11)

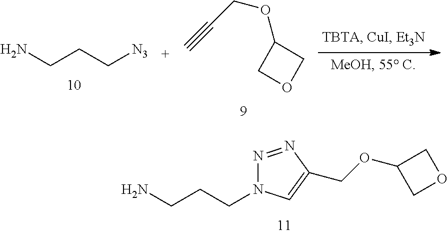

A mixture of 3-(prop-2-yn-1-yloxy)oxetane (9, 7.96 g, 71 mmol, 1.0 eq), 3-azidopropan-1-amine (10, 7.82 g, 78 mmol, 1.1 eq), Tris[(1-benzyl-1H-1,2,3-triazol-4-yl)methyl]-amine (8.29 g, 15.6 mmol, 0.22 eq), Copper Iodide (1.35 g, 7.1 mmol, 0.1 eq), and Triethylamine (2.47 mL, 17.8 mmol, 0.25 eq) in Methanol (80 mL) was warmed to 55° C. and stirred over night under Nitrogen atmosphere. The reaction mixture was cooled to room temperature, Celite (20 g) was added, and concentrated under reduced pressure. The crude product was purified over silica gel (220 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 15% to afford 3-(4-((oxetan-3-yloxy)methyl)-1H-1,2,3-triazol-1-yl)propan-1-amine (11, 11.85 g, 79%) as a yellow oil. LCMS m/z: [M+H]⁺ Calcd for C₉H₁₆N₄O₂ 213.1; Found 213.0.

### Experimental Procedure for N-(3-(4-((oxetan-3-yloxy)methyl)-1H-1,2,3-triazol-1-yl)propyl)methacrylamide (12)

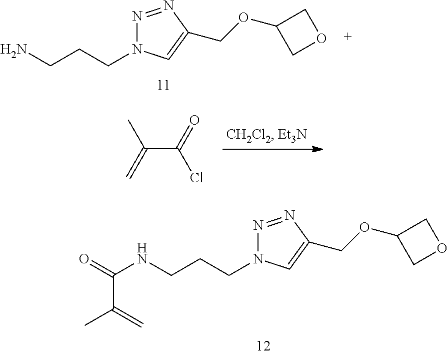

A solution of 3-(4-((oxetan-3-yloxy)methyl)-1H-1,2,3-triazol-1-yl)propan-1-amine (11, 3.94 g, 18.56 mmol, 1.0 eq) and triethylamine (3.1 mL, 22.28 mmol, 1.2 eq) in CH₂Cl₂ (100 mL) was cooled to 0° C. with an ice-bath and methacryloyl chloride (1.99 mL, 20.42 mmol, 1.1 eq) was added in a dropwise fashion. The reaction was stirred over night while allowed to warm to room temperature. 20 grams of Celite were added and the solvent was removed under reduced pressure. The residue was purified by silica gel chromatography (220 g) using dichloromethane/methanol as mobile phase. The concentration of methanol was gradually increased from 0% to 5% to afford N-(3-(4-((oxetan-3-yloxy)methyl)-1H-1,2,3-triazol-1-yl)propyl)methacrylamide (12, 3.22 g, 62% yield) as a solid. LCMS m/z: [M+H]⁺ Calcd for C₁₃H₂₀N₄O₃ 281.2; Found 281.0.

### Experimental Procedure for N-(4-(1H-1,2,3-triazol-1-yl)benzyl) methacrylamide (14)

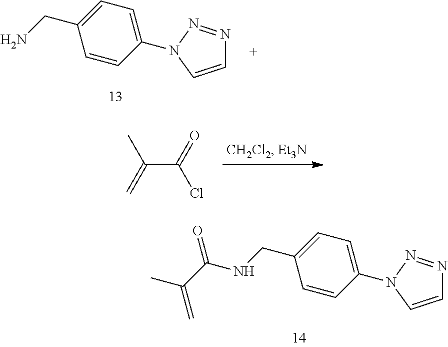

To a solution of (4-(1H-1,2,3-triazol-1-yl)phenyl)methanamine (13, obtained from WuXi, 1.2 g, 5.70 mmol, 1.0 eq) and triethylamine (15 mL, 107.55 mmol, 18.9 eq) in CH₂Cl₂ (100 mL) was slowly added methacryloyl chloride (893 mg, 8.54 mmol, 1.5 eq) in a dropwise fashion. The reaction was stirred over night. 20 grams of Celite were added and the solvent was removed under reduced pressure. The residue was purified by silica gel chromatography using dichloromethane/(methanol containing 12% (v/v) aqueous ammonium hydroxide) as mobile phase. The concentration of (methanol containing 12% (v/v) aqueous ammonium hydroxide) was gradually increased from 0% to 1.25% to afford N-(4-(1H-1,2,3-triazol-1-yl)benzyl) methacrylamide (14, 1.38 g, 40% yield).

### Experimental Procedure for (4-(4-(((tetrahydro-2H-pyran-2-yl)oxy)methyl)-1H-1,2,3-triazol-1-yl)phenyl)methanamine (15)

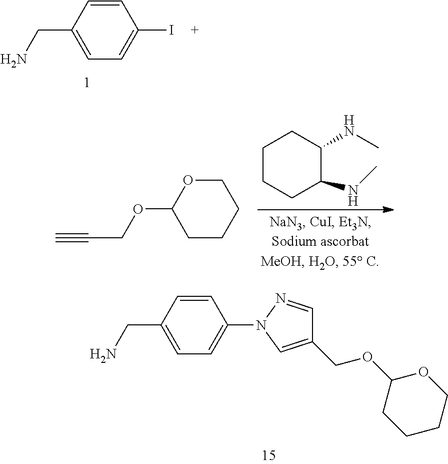

A mixture of (4-iodophenyl)methanamine hydrochloride (5.0 g, 18.55 mmol, 1.0 eq), (1S,2S)—N1,N2-dimethylcyclohexane-1,2-diamine (0.59 mL 3.71 mmol, 0.2 eq), Sodium ascorbate (368 mg, 1.86 mmol, 0.1 eq), Copper Iodide (530 mg, 2.78 mmol, 0.15 eq), Sodium azide (2.41 g, 37.1 mmol, 2.0 eq), Et₃N (3.11 mL, 22.26 mmol, 1.2 eq) and 2-(prop-2-yn-1-yloxy)tetrahydro-2H-pyran (2.6 g, 18.55 mmol, 1.0 eq) in Methanol (50 mL) and water (12 mL) were purged with Nitrogen for 5 minutes and heated to 55° C. for over night. The reaction mixture was cooled to room temperature and filtered through 413 filter paper. Celite was added and the solvent was removed under reduced pressure and the residue was purified over silica gel (120 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 6.25% to afford (4-(4-(((tetrahydro-2H-pyran-2-yl)oxy)methyl)-1H-1,2,3-triazol-1-yl)phenyl)methanamine (15, 3.54 g, 66%) as a white solid. LCMS m/z: [M+H]⁺ Calcd for C₁₅H₂₀N₄O₂ 289.2; Found 289.2.

### Experimental Procedure for N-(4-(4-(((tetrahydro-2H-pyran-2-yl)oxy)methyl)-1H-1,2,3-triazol-1-yl)benzyl)methacrylamide (16)

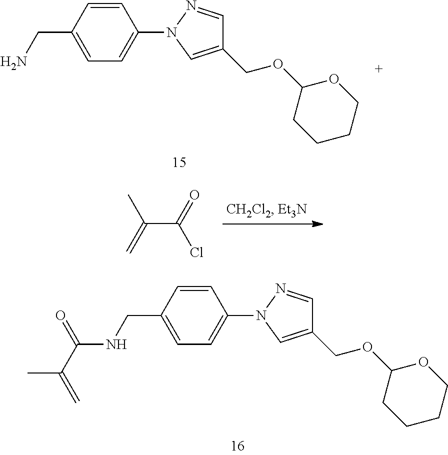

A solution of (4-(4-(((tetrahydro-2H-pyran-2-yl)oxy)methyl)-1H-1,2,3-triazol-1-yl)phenyl)methanamin (15, 3.46 g, 12.00 mmol, 1.0 eq) and triethylamine (2.01 mL, 14.40 mmol, 1.2 eq) in CH₂Cl₂ (40 mL) was cooled to 0° C. with an ice-bath and methacryloyl chloride (1.23 mL, 12.60 mmol, 1.05 eq, diluted in 5 mL of CH₂Cl₂) was added in a dropwise fashion. The cooling bath was removed and the reaction was stirred for 4 h. 20 grams of Celite was added and the solvent was removed under reduced pressure. The residue was purified by silica gel chromatography (80 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 3.75% to afford N-(4-(4-(((tetrahydro-2H-pyran-2-yl)oxy)methyl)-1H-1,2,3-triazol-1-yl)benzyl)methacrylamide (16, 2.74 g, 64% yield) as a white solid. LCMS m/z: [M+H]⁺ Calcd for C₁₉H₂₄N₄O₃ 357.2; Found 357.3.

### Experimental Procedure for N-(4-(4-(hydroxymethyl)-1H-1,2,3-triazol-1-yl)benzyl)methacrylamide (17)

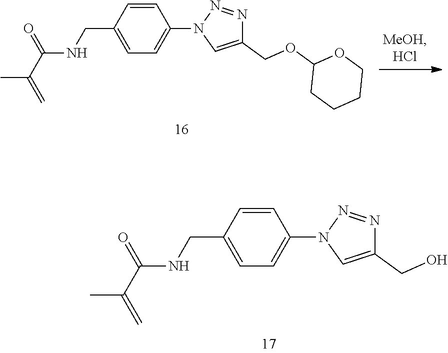 [SMILES: C=C(C)C(=O)NCc1ccc(-n2cc(COC3CCCCO3)nn2)cc1 ; C=C(C)C(=O)NCc1ccc(-n2cc(CO)nn2)cc1]

A solution of N-(4-(4-(hydroxymethyl)-1H-1,2,3-triazol-1-yl)benzyl)methacrylamide (16, 1.2 g, 3.37 mmol, 1.0 eq) was dissolved in Methanol (6 mL) and HCl (1N, aq., 9 mL) for over night at room temperature. Celite was added and the solvent was removed under reduced pressure. The crude product was purified over silica gel chromatography (24 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 12.5% to afford N-(4-(4-(hydroxymethyl)-1H-1,2,3-triazol-1-yl)benzyl)methacrylamide (17, 0.85 g, 92% yield) as a white solid. LCMS m/z: [M+H]⁺ Calcd for C₁₄H₁₆N₄O₂ 273.1; Found 273.1.

### Experimental Procedure for (4-(((tetrahydro-2H-pyran-2-yl)oxy)methyl)benzyl)carbamate (19)

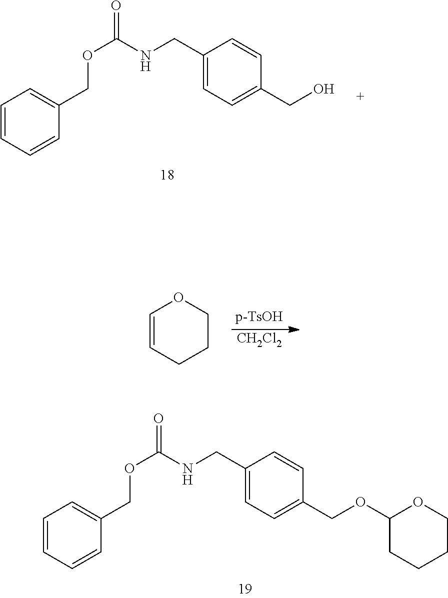

Benzyl (4-(hydroxymethyl)benzyl)carbamate (2.71 g, 10 mmol, 1 eq), 3,4-dihydro-2H-pyran (1.81 mL, 20 mmol, 2 eq), p-Toluenesulfonic acid monohydrate (285 mg, 1.5 mmol, 0.15 eq) in dichloromethane (100 mL) were stirred at room temperature over night. Celite was added and the solvent was removed under reduced pressure. The crude product was purified over silica gel (24 g) using Hexanes/EtOAc as eluent starting at 100% Hexanes and increasing the concentration of EtOAc gradually to 100% to afford benzyl (4-(((tetrahydro-2H-pyran-2-yl)oxy)methyl)benzyl)-carbamate (19, 2.4 g, 68%) as a colorless oil. LCMS m/z: [M+Na]⁺ Calcd for C₂₁H₂₅NO₄ 378.17 Found 378.17.

### Experimental Procedure for (4-(((tetrahydro-2H-pyran-2-yl)oxy)methyl)-phenyl)methanamine (20)

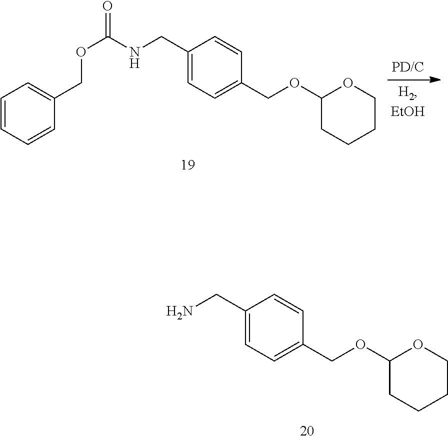 [SMILES: O=C(NCc1ccc(COC2CCCCO2)cc1)OCc1ccccc1 ; NCc1ccc(COC2CCCCO2)cc1]

(4-(((tetrahydro-2H-pyran-2-yl)oxy)methyl)benzyl)carbamate (19, 1.5 g, 4.2 mmol, 1 eq), Palladium on carbon (160 mg, 10 wt. %) in EtOH was briefly evacuated and then Hydrogen was added via a balloon and the mixture was stirred for 1 hour at room temperature. Celite was added and the solvent was removed under reduced pressure. The crude product was purified over silica gel (12 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 25% to afford (4-(((tetrahydro-2H-pyran-2-yl)oxy)methyl)phenyl)methanamine (20, 890 mg, 95%) as a colorless oil. LCMS m/z: [M+H]⁺ Calcd for C₁₃H₁₉NO₂ 222.15 Found 222.14.

### Experimental Procedure for N-(4-(((tetrahydro-2H-pyran-2-yl)oxy)methyl)benzyl)-methacrylamide (21)

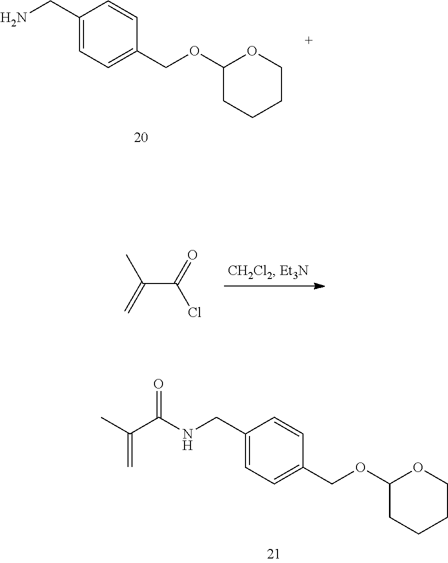

A solution of (4-(((tetrahydro-2H-pyran-2-yl)oxy)methyl)phenyl)methanamine (20, 0.5 g, 2.26 mmol, 1.0 eq) and triethylamine (0.47 mL, 3.39 mmol, 1.5 eq) in CH₂Cl₂ (10 mL) were briefly evacuated and flushed with Nitrogen. Methacryloyl chloride (0.33 mL, 3.39 mmol, 1.5 eq) was added in a dropwise fashion. The reaction mixture was stirred over night at room temperature. 10 grams of Celite was added and the solvent was removed under reduced pressure. The residue was purified by silica gel chromatography (12 g) using Hexanes/EtOAc as eluent starting at 100% Hexanes and increasing the concentration of EtOAc gradually to 100% to afford N-(4-(((tetrahydro-2H-pyran-2-yl)oxy)methyl)benzyl)methacrylamide (21, 0.47 g, 72% yield) as a colorless solid. LCMS m/z: [M+Na]⁺ Calcd for C₁₇H₂₃NO₃ 312.16; Found 312.17.

### Experimental Procedure (4-(4-(2-((tetrahydro-2H-pyran-2-yl)oxy)ethyl)-1H-1,2,3-triazol-1-yl)phenyl)methanamine (22)

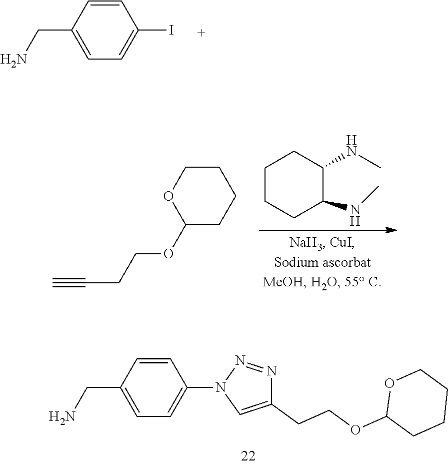

A mixture of (4-iodophenyl)methanamine (5.0 g, 21.45 mmol, 1.0 eq), (1S,2S)—N1,N2-dimethylcyclohexane-1,2-diamine (0.44 mL 2.79 mmol, 0.13 eq), Sodium ascorbate (425 mg, 2.15 mmol, 0.1 eq), Copper Iodide (409 mg, 2.15 mmol, 0.1 eq), Sodium azide (2.79 g, 42.91 mmol, 2.0 eq), and 2-(but-3-yn-1-yloxy)tetrahydro-2H-pyran (3.36 mL, 21.45 mmol, 1.0 eq) in Methanol (20 mL) and water (5 mL) were purged with Nitrogen for 5 minutes and heated to 55° C. for over night. The reaction mixture was cooled to room temperature and filtered through 413 filter paper. Celite (10 g) was added and the solvent was removed under reduced pressure and the residue was purified over silica gel (220 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 5% to afford (4-(4-(2-((tetrahydro-2H-pyran-2-yl)oxy)ethyl)-1H-1,2,3-triazol-1-yl)phenyl)methanamine (22, 3.15 g, 49%) as a solid. LCMS m/z: [M+H]⁺ Calcd for C₁₆H₂₂N₄O₂ 303.18; Found 303.18.

### Experimental Procedure for N-(4-(4-(2-((tetrahydro-2H-pyran-2-yl)oxy)ethyl)-1H-1,2,3-triazol-1-yl)benzyl)methacrylamide (23)

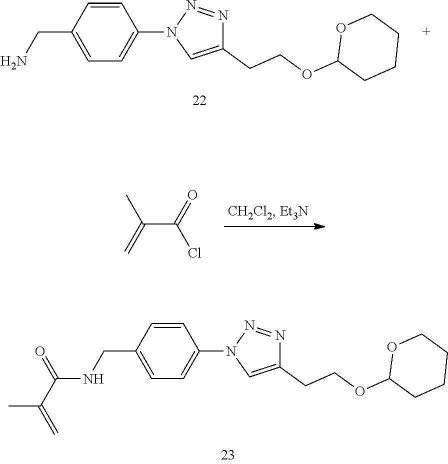

A solution of (4-(4-(2-((tetrahydro-2H-pyran-2-yl)oxy)ethyl)-1H-1,2,3-triazol-1-yl)phenyl)methanamine (22, 3.10 g, 10.25 mmol, 1.0 eq) and triethylamine (1.71 mL, 12.30 mmol, 1.2 eq) in CH₂Cl₂ (55 mL) was cooled to 0° C. with an ice-bath and methacryloyl chloride (1.05 mL, 12.30 mmol, 1.2 eq, diluted in 5 mL of CH₂Cl₂) was added in a dropwise fashion. The cooling bath was removed and the reaction was stirred for 4 h. 8 grams of Celite was added and the solvent was removed under reduced pressure. The residue was purified by silica gel chromatography (80 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 2.5% to afford N-(4-(4-(2-((tetrahydro-2H-pyran-2-yl)oxy)ethyl)-1H-1,2,3-triazol-1-yl)benzyl)methacrylamide (23, 2.06 g, 54% yield) as a white solid. LCMS m/z: [M+H]⁺ Calcd for C₂₀H₂₆N₄O₃ 371.2078; Found 371.2085.

### Experimental Procedure (4-(1-(2-((tetrahydro-2H-pyran-2-yl)oxy)ethyl)-1H-1,2,3-triazol-4-yl)phenyl)methanamine (24)

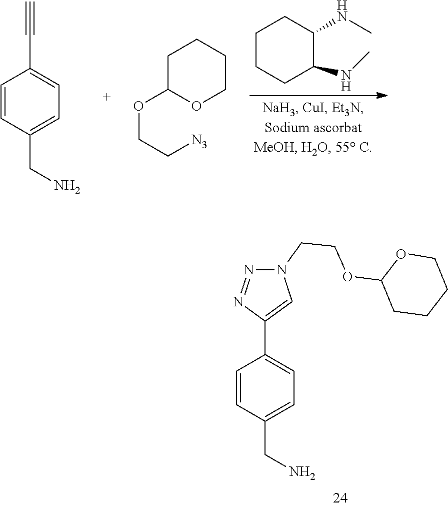

A mixture of (4-ethynylphenyl)methanamine (2.36 g, 18.00 mmol, 1.0 eq), (1S,2S)—N1,N2-dimethylcyclohexane-1,2-diamine (0.56 mL, 3.60 mmol, 0.2 eq), Sodium ascorbate (357 mg, 1.80 mmol, 0.1 eq), Copper Iodide (514 mg, 2.70 mmol, 0.15 eq), and 2-(2-azidoethoxy)tetrahydro-2H-pyran (3.08, 18.00 mmol, 1.0 eq) in Methanol (24 mL) and water (6 mL) were purged with Nitrogen for 5 minutes and heated to 55° C. for over night. The reaction mixture was cooled to room temperature and filtered over Celite and rinsed with MeOH (3×50 mL). The solvent was removed under reduced pressure and the residue was redissolved in dichloromethane, Celite (20 g) was added and the solvent was removed under reduced pressure and the residue was purified over silica gel (120 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 25% to afford (4-(1-(2-((tetrahydro-2H-pyran-2-yl)oxy)ethyl)-1H-1,2,3-triazol-4-yl)phenyl)methanamine (24, 3.51 g, 64%) as a yellowish oil. LCMS m/z: [M+H]⁺ Calcd for C₁₆H₂₂N₄O₂ 303.1816; Found 303.1814.

### Experimental Procedure for N-(4-(1-(2-((tetrahydro-2H-pyran-2-yl)oxy)ethyl)-1H-1,2,3-triazol-4-yl)benzyl)methacrylamide (25)

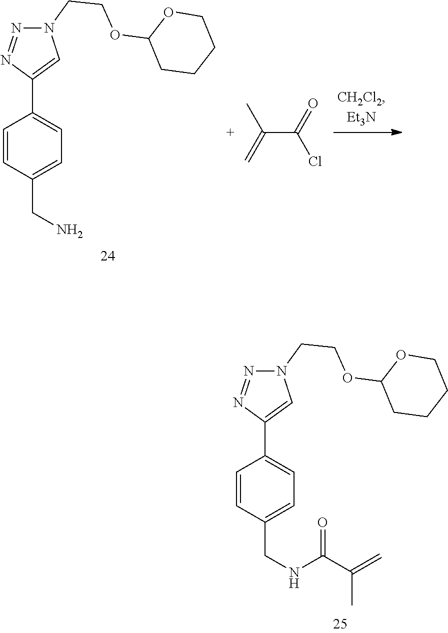

A solution of (4-(1-(2-((tetrahydro-2H-pyran-2-yl)oxy)ethyl)-1H-1,2,3-triazol-4-yl)phenyl)methanamine (24, 1.5 g, 4.96 mmol, 1.0 eq) and triethylamine (1.04 mL, 7.44 mmol, 1.5 eq) in CH₂Cl₂ (30 mL) were briefly evacuated and flushed with Nitrogen. Methacryloyl chloride (0.72 mL, 7.44 mmol, 1.5 eq) was added in a dropwise fashion. The reaction mixture was stirred for 2 h at room temperature. 10 grams of Celite was added and the solvent was removed under reduced pressure. The residue was purified by silica gel chromatography (40 g) using Hexanes/EtOAc as eluent starting at 100% Hexanes and increasing the concentration of EtOAc gradually to 100% to afford N-(4-(1-(2-((tetrahydro-2H-pyran-2-yl)oxy)ethyl)-1H-1,2,3-triazol-4-yl)benzyl)methacrylamide (25, 0.9 g, 49% yield) as a colorless solid. LCMS m/z: [M+Na]⁺ Calcd for C₂₀H₂₆N₅O₃ 371.2078; Found 371.2076.

### Experimental Procedure for 1-(4-(4-(((tetrahydro-2H-pyran-2-yl)oxy)methyl)-1H-1,2,3-triazol-1-yl)phenyl)ethan-1-amine (26)

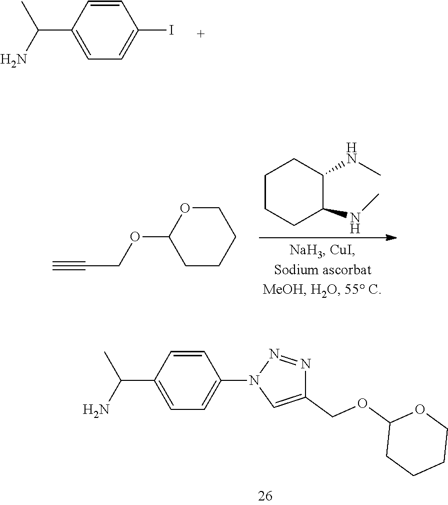

A mixture of 1-(4-iodophenyl)ethan-1-amine hydrochloride (1.0 g, 4.05 mmol, 1.0 eq), (1S,2S)—N1,N2-dimethylcyclohexane-1,2-diamine (0.08 mL 0.53 mmol, 0.13 eq), Sodium ascorbate (80 mg, 0.40 mmol, 0.1 eq), Copper Iodide (77 mg, 0.40 mmol, 0.1 eq), Sodium azide (526 g, 8.09 mmol, 2.0 eq), and 2-(prop-2-yn-1-yloxy)tetrahydro-2H-pyran (0.57 g, 4.05 mmol, 1.0 eq) in Methanol (9 mL) and water (1 mL) were purged with Nitrogen for 5 minutes and heated to 55° C. for over night. The reaction mixture was cooled to room temperature and the solvent was removed under reduced pressure. The residue was redissolved in dichloromethane and filtered over a plug of Celite. Celite was added to the filtrate and the solvent was removed under reduced pressure. The residue was purified over silica gel (40 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 5% to afford 1-(4-(4-(((tetrahydro-2H-pyran-2-yl)oxy)methyl)-1H-1,2,3-triazol-1-yl)phenyl)ethan-1-amine (26, 0.62 g, 51%) as a yellowish solid. LCMS m/z: [M+H]⁺ Calcd for C₁₆H₂₂N₄O₂ 303.2; Found 303.2.

### Experimental Procedure for N-(1-(4-(4-(((tetrahydro-2H-pyran-2-yl)oxy) methyl)-1H-1,2,3-triazol-1-yl)phenyl)ethyl)methacrylamide (27)

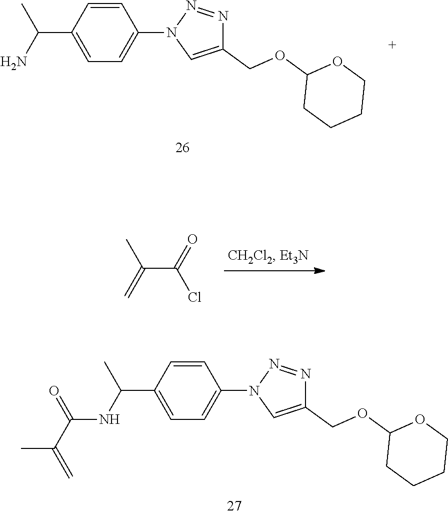

A solution of 1-(4-(4-(((tetrahydro-2H-pyran-2-yl)oxy)methyl)-1H-1,2,3-triazol-1-yl)phenyl)ethan-1-amine (26, 0.52 g, 1.7 mmol, 1.0 eq) and triethylamine (0.29 mL, 2.1 mmol, 1.2 eq) in CH₂Cl₂ (11 mL) was cooled to 0° C. with an ice-bath and methacryloyl chloride (0.18 mL, 1.8 mmol, 1.05 eq, diluted in 11 mL of CH₂Cl₂) was added in a dropwise fashion. The cooling bath was removed and the reaction was stirred for 4 h. 5 grams of Celite was added and the solvent was removed under reduced pressure. The residue was purified by silica gel chromatography (40 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 2.5% to afford N-(1-(4-(4-(((tetrahydro-2H-pyran-2-yl)oxy) methyl)-1H-1,2,3-triazol-1-yl)phenyl)ethyl)methacrylamide (27, 0.49 g, 76% yield) as a white solid. LCMS m/z: [M+H]⁺ Calcd for C₂₀H₂₆N₄O₃ 371.2078; Found 371.2087.

### Experimental Procedure for (4-(4-(((tetrahydro-2H-pyran-2-yl)oxy)methyl)-1H-1,2,3-triazol-1-yl)-2-(trifluoromethyl)phenyl)methanamine (28)

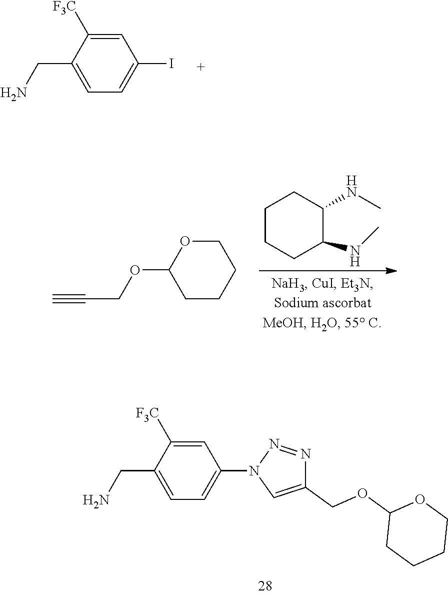

A mixture of (4-iodo-2-(trifluoromethyl)phenyl)methanamine (3.0 g, 9.97 mmol, 1.0 eq), (1S,2S)—N1,N2-dimethylcyclohexane-1,2-diamine (0.31 mL 1.99 mmol, 0.2 eq), Sodium ascorbate (197 mg, 1.00 mmol, 0.1 eq), Copper Iodide (285 mg, 1.49 mmol, 0.15 eq), Sodium azide (1.30 g, 19.93 mmol, 2.0 eq), Et₃N (1.67 mL, 11.96 mmol, 1.2 eq) and 2-(prop-2-yn-1-yloxy)tetrahydro-2H-pyran (1.40 g, 9.97 mmol, 1.0 eq) in Methanol (24 mL) and water (6 mL) were purged with Nitrogen for 5 minutes and heated to 55° C. for over night. The reaction mixture was cooled to room temperature and filtered through a plug of Celite and rinsed with Methanol (3×50 mL). Celite was added to the filtrate and the solvent was removed under reduced pressure. The residue was purified over silica gel (120 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 25% to afford (4-(4-(((tetrahydro-2H-pyran-2-yl)oxy)methyl)-1H-1,2,3-triazol-1-yl)-2-(trifluoromethyl)phenyl)methanamine (28, 2.53 g, 71%) as a green oil. LCMS m/z: [M+H]⁺ Calcd for C₁₆H₁₉N₄O₂F₃ 357.2; Found 357.1.

### Experimental Procedure for N-(4-(4-(((tetrahydro-2H-pyran-2-yl)oxy)methyl)-1H-1,2,3-triazol-1-yl)-2(trifluoromethyl)benzyl) methacrylamide (29)

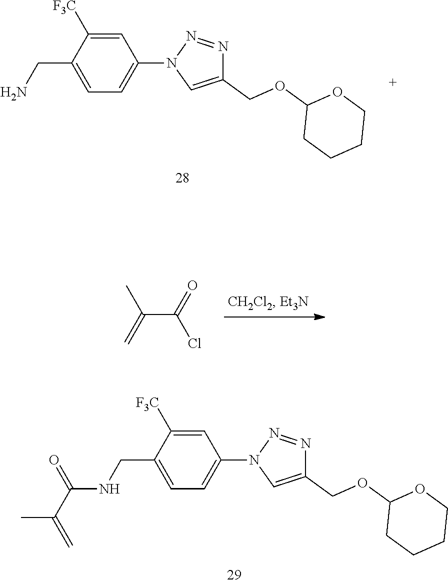

A solution of (4-(4-(((tetrahydro-2H-pyran-2-yl)oxy)methyl)-1H-1,2,3-triazol-1-yl)-2-(trifluoromethyl)phenyl) methanamine (28, 1.0 g, 2.81 mmol, 1.0 eq) and triethylamine (0.59 mL, 4.21 mmol, 1.5 eq) in CH₂Cl₂ (25 mL) were briefly evacuated and flushed with Nitrogen. Methacryloyl chloride (0.41 mL, 4.21 mmol, 1.5 eq) was added in a dropwise fashion. The reaction mixture was stirred for 6 h at room temperature. 10 grams of Celite was added and the solvent was removed under reduced pressure. The residue was purified by silica gel chromatography (40 g) using Hexanes/EtOAc as eluent starting at 100% Hexanes and increasing the concentration of EtOAc gradually to 100% to afford N-(4-(4-(((tetrahydro-2H-pyran-2-yl)oxy)methyl)-1H-1,2,3-triazol-1-yl)-2(trifluoromethyl)benzyl) methacrylamide (29, 0.65 g, 55% yield) as a colorless solid. LCMS m/z: [M+H]⁺ Calcd for C₂₀H₂₃N₄O₃F₃ 425.2; Found 425.1.

### Experimental Procedure for 3-(4-(((tetrahydro-2H-pyran-2-yl)oxy)methyl)-1H-1,2,3-triazol-1-yl)propan-1-amine (30)

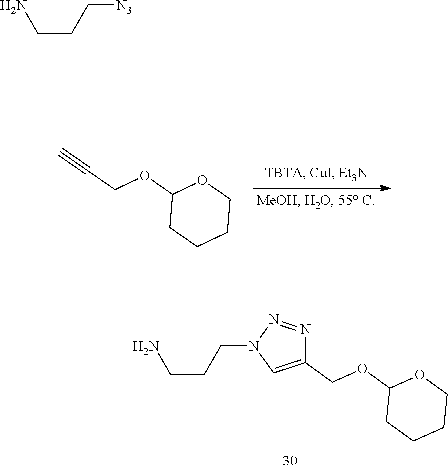

A mixture of 3-azidopropan-1-amine hydrochloride (1.5 g, 14.98 mmol, 1.0 eq), Tris[(1-benzyl-1H-1,2,3-triazol-4-yl)methyl]-amine (1.99 g, 3.75 mmol, 0.25 eq), Copper Iodide (0.29 g, 1.50 mmol, 0.1 eq), and Triethylamine (0.52 mL, 3.75 mmol, 0.25 eq) in Methanol (50 mL) and water (6 mL) were purged with Nitrogen for 5 minutes and cooled to 0 C. 2-(prop-2-yn-1-yloxy)tetrahydro-2H-pyran (2.10 g, 14.98 mmol, 1.0 eq) was added and the reaction mixture was warmed to 55° C. and stirred over night under Nitrogen atmosphere. The reaction mixture was cooled to room temperature, filtered over a plug of Celite and rinsed with Methanol (3×50 mL). Celite (20 g) was added to the filtrate the solvent was removed under reduced pressure. The residue was purified over silica gel (120 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 20% to afford 3-(4-(((tetrahydro-2H-pyran-2-yl)oxy)methyl)-1H-1,2,3-triazol-1-yl)propan-1-amine (30, 2.36 g, 66%). LCMS m/z: [M+H]⁺ Calcd for C₁₁H₂₀N₄O₂ 241.2; Found 241.2.

### Experimental Procedure for N-(3-(4-(((tetrahydro-2H-pyran-2-yl)oxy)methyl)-1H-1,2,3-triazol-1-yl)propyl)methacrylamide (31)

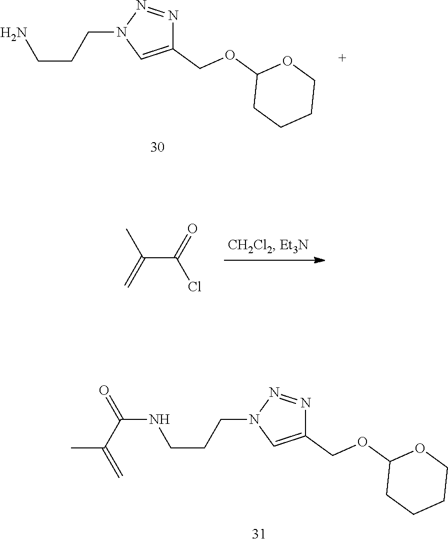

A solution of 3-(4-(((tetrahydro-2H-pyran-2-yl)oxy)methyl)-1H-1,2,3-triazol-1-yl)propan-1-amine (30, 1.0 g, 4.16 mmol, 1.0 eq) and triethylamine (0.58 mL, 4.16 mmol, 1.0 eq) in CH₂Cl₂ (20 mL) were briefly evacuated and flushed with Nitrogen. Methacryloyl chloride (0.40 mL, 4.16 mmol, 1.0 eq) was added in a dropwise fashion. The reaction mixture was stirred at room temperature over night. 10 grams of Celite was added and the solvent was removed under reduced pressure. The residue was purified by silica gel chromatography (40 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 20% to afford N-(3-(4-(((tetrahydro-2H-pyran-2-yl)oxy)methyl)-1H-1,2,3-triazol-1-yl)propyl)methacrylamide (31, 0.96 g, 75% yield) as a colorless oil. LCMS m/z: [M+H]⁺ Calcd for C₁₅H₂₄N₄O₃ 309.2; Found 309.4.

### Experimental Procedure for (4-(4-((oxetan-3-yloxy)methyl)-1H-1,2,3-triazol-1-yl)phenyl)methanamine (32)

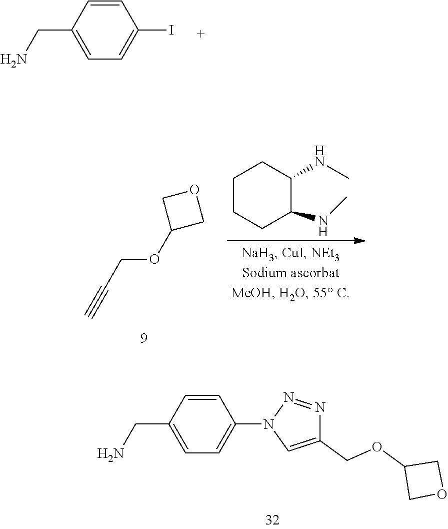

A mixture of (4-iodophenyl)methanamine hydrochloride (2.64 g, 9.80 mmol, 1.0 eq), (1S,2S)—N1,N2-dimethylcyclohexane-1,2-diamine (0.31 mL 1.96 mmol, 0.2 eq), Sodium ascorbate (198 mg, 0.98 mmol, 0.1 eq), Copper Iodide (279 mg, 1.47 mmol, 0.15 eq), Sodium azide (1.27 g, 19.59 mmol, 2.0 eq), Et₃N (1.64 mL, 11.75 mmol, 1.2 eq) and 3-(prop-2-yn-1-yloxy)oxetane (9, 1.10 g, 9.80 mmol, 1.0 eq) in Methanol (24 mL) and water (6 mL) were purged with Nitrogen for 5 minutes and heated to 55° C. for over night. The reaction mixture was cooled to room temperature and filtered through a plug of Celite and rinsed with Methanol (3×50 mL). Celite was added to the filtrate and the solvent was removed under reduced pressure. The residue was purified over silica gel (120 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 25% to afford (4-(4-((oxetan-3-yloxy)methyl)-1H-1,2,3-triazol-1-yl)phenyl)methanamine (32, 1.43 g, 56%) as an oil. LCMS m/z: [M+H]⁺ Calcd for C₁₃H₁₆N₄O₂ 261.1346; Found 261.1342.

### Experimental Procedure for N-(4-(4-((oxetan-3-yloxy)methyl)-1H-1,2,3-triazol-1-yl)benzyl)methacrylamide (33)

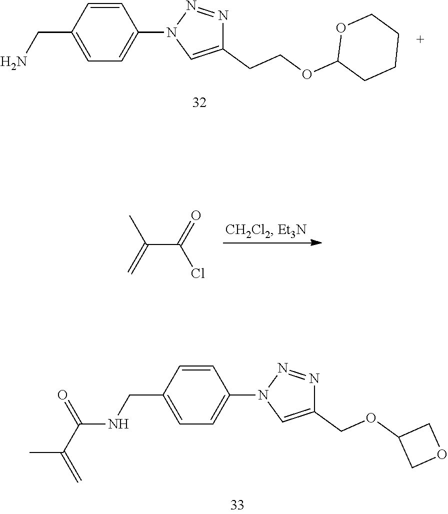

A solution of (4-(4-((oxetan-3-yloxy)methyl)-1H-1,2,3-triazol-1-yl)phenyl)methanamine (32, 0.58 g, 2.23 mmol, 1.0 eq) and triethylamine (0.47 mL, 3.34 mmol, 1.5 eq) in CH₂Cl₂ (20 mL) were briefly evacuated and flushed with Nitrogen. Methacryloyl chloride (0.32 mL, 3.34 mmol, 1.5 eq) was added in a dropwise fashion. The reaction mixture was stirred for 6 h at room temperature. 10 grams of Celite was added and the solvent was removed under reduced pressure. The residue was purified by silica gel chromatography (24 g) using Hexanes/EtOAc as eluent starting at 100% Hexanes and increasing the concentration of EtOAc gradually to 100% to afford N-(4-(4-((oxetan-3-yloxy)methyl)-1H-1,2,3-triazol-1-yl)benzyl)methacrylamide (33, 0.48 g, 66% yield) as a colorless solid. LCMS m/z: [M+H]⁺ Calcd for C₁₇H₂₀N₄O₃ 329.1608; Found 329.1611.

### Experimental Procedure for ethyl 1-(2-methacrylamidoethyl)-1H-imidazole-4-carboxylate (35)

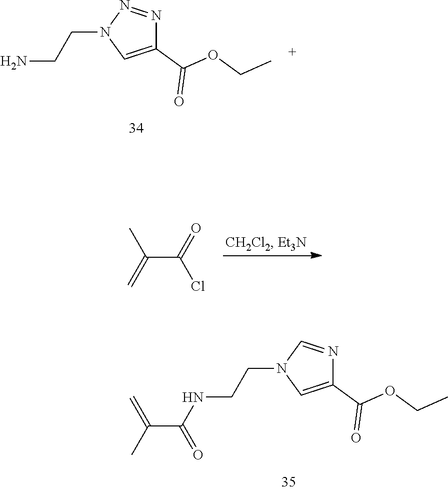

A solution of ethyl 1-(2-aminoethyl)-1H-imidazole-4-carboxylate (34, 2.0 g, 10.91 mmol, 1.0 eq) and triethylamine (3.80 mL, 27.29 mmol, 2.5 eq) in CH₂Cl₂ (20 mL) were briefly evacuated and flushed with Nitrogen. Methacryloyl chloride (1.60 mL, 16.37 mmol, 1.5 eq) was added in a dropwise fashion. The reaction mixture was stirred for 3 h at room temperature. 15 grams of Celite was added and the solvent was removed under reduced pressure. The residue was purified by silica gel chromatography (40 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 25% to afford ethyl 1-(2-methacrylamidoethyl)-1H-imidazole-4-carboxylate (35, 1.28 g, 47% yield) as a colorless solid. LCMS m/z: [M+H]⁺ Calcd for C₁₂H₁₇N₃O₃ 252.1; Found 252.1.

### Experimental Procedure for N-(4-(1,1-dioxidothiomorpholino)benzyl) methacrylamide (37)

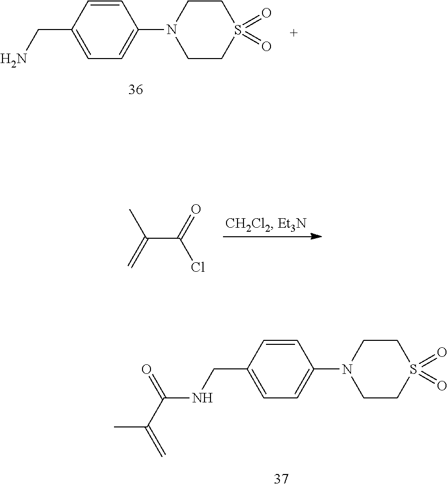

To a solution of 4-(4-(aminomethyl)phenyl)thiomorpholine 1,1-dioxide hydrochloride (36, 1.15 g, 4.15 mmol, 1.0 eq) and triethylamine (1.39 mL, 9.97 mmol, 2.4 eq) in CH₂Cl₂ (80 mL) was added a solution of methacryloyl chloride (0.43 mL, 4.36 mmol, 1.05 eq, in CH₂Cl₂, 5 mL) in a dropwise fashion. The reaction mixture was stirred for 22 h at room temperature. 8 grams of Celite was added and the solvent was removed under reduced pressure. The residue was purified by silica gel chromatography (80 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 3.75% to afford N-(4-(1,1-dioxidothiomorpholino)benzyl) methacrylamide (37, 0.32 g, 25% yield) as a solid.

### Experimental Procedure for N-methyl-N-(2-(methylsulfonyl)ethyl)prop-2-yn-1-amine (38)

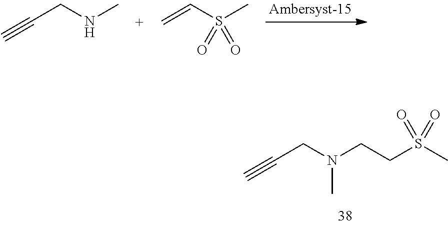

To a mixture of 1-methylsulfonylethylene (4.99 g, 47.03 mmol, 4.13 mL) and Amberlyst-15 ((30% w/w)), N-methylprop-2-yn-1-amine (2.6 g, 37.62 mmol) was added in a dropwise fashion. The mixture was stirred at room temperature for 12 hours. The catalyst was removed by filtration and the filtrate was concentrated under reduced pressure to afford: N-methyl-N-(2-(methylsulfonyl)ethyl)prop-2-yn-1-amine (38, 6.43 g, 98%) as an oil. LCMS m/z: [M+H]⁺ Calcd for C₇H₁₃NSO₂ 176.11; Found 176.1.

### Experimental Procedure for N-((1-(2-(2-(2-(2-aminoethoxy)ethoxy)ethoxy) ethyl)-1H-1,2,3-triazol-4-yl)methyl)-N-methyl-2-(methylsulfonyl)ethan-1-amine (40)

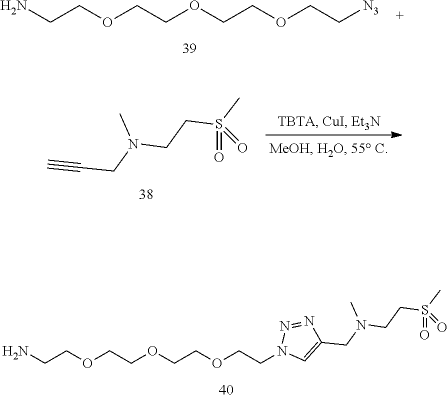

A mixture of N-methyl-N-(2-(methylsulfonyl)ethyl)prop-2-yn-1-amine (38, 5.02 g, 28.64 mmol, 1.25 eq), Tris[(1-benzyl-1H-1,2,3-triazol-4-yl)methyl]-amine (3.04 g, 5.73 mmol, 0.25 eq), Copper Iodide (436 mg, 2.29 mmol, 0.1 eq), and Triethylamine (0.8 mL, 5.7 mmol, 0.25 eq) in Methanol (50 mL) and water (6 mL) was evacuated and flushed with Nitrogen (3 times) and cooled with an ice bath. 2-(2-(2-(2-azidoethoxy)ethoxy)ethoxy)ethan-1-amine (39, 5.02 g, 22.91 mmol, 1.0 eq) was added in a dropwise fashion, the cooling bath was removed and the mixture was stirred for 5 minutes. The reaction was warmed to 55° C. and stirred over night under Nitrogen atmosphere. The reaction mixture was cooled to room temperature, Celite (20 g) was added, and concentrated under reduced pressure. The crude product was purified over silica gel (220 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 25% to afford N-((1-(2-(2-(2-(2-aminoethoxy)ethoxy)ethoxy)ethyl)-1H-1,2,3-triazol-4-yl)methyl)-N-methyl-2-(methylsulfonyl)ethan-1-amine (40, 4.98 g, 55%) as an oil. LCMS m/z: [M+H]⁺ Calcd for C₁₅H₃₁N₅O₅S 394.2; Found 394.2.

### Experimental Procedure N-(2-(2-(2-(2-(4-((methyl(2-(methylsulfonyl)ethyl) amino)methyl)-1H-1,2,3-triazol-1-yl)ethoxy)ethoxy)ethoxy) ethyl)methacrylamide (41)

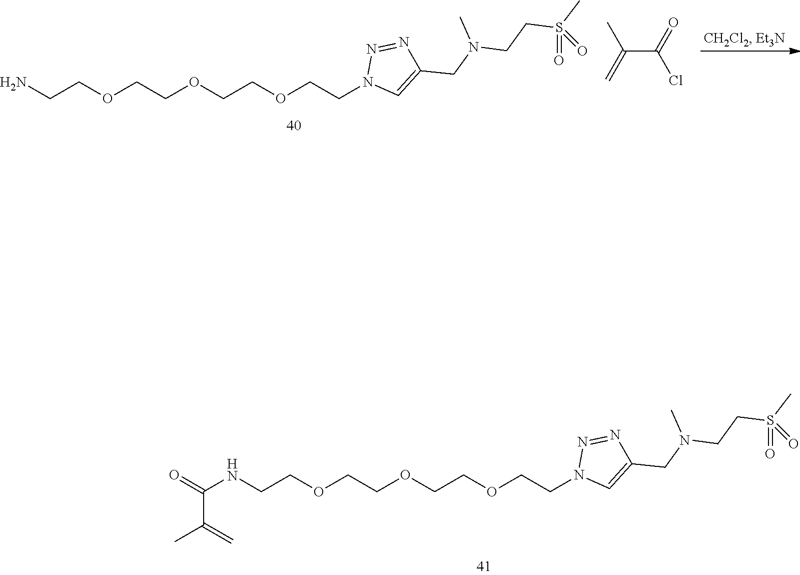

To a solution of N-((1-(2-(2-(2-(2-aminoethoxy)ethoxy)ethoxy)ethyl)-1H-1,2,3-triazol-4-yl)methyl)-N-methyl-2-(methylsulfonyl)ethan-1-amine (40, 1.0 g, 2.54 mmol, 1.0 eq) and triethylamine (0.43 mL, 3.05 mmol, 1.2 eq) in CH₂Cl₂ (15 mL) was added a solution of methacryloyl chloride (0.30 mL, 3.05 mmol, 1.5 eq) in a dropwise fashion. The reaction mixture was stirred for 5 h at room temperature. Celite was added and the solvent was removed under reduced pressure. The residue was purified by silica gel chromatography (40 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 12.5% to afford N-(2-(2-(2-(2-(4-((methyl(2-(methylsulfonyl)ethyl) amino)methyl)-1H-1,2,3-triazol-1-yl)ethoxy)ethoxy)ethoxy) ethyl)methacrylamide (41, 0.86 g, 73% yield) as an oil. LCMS m/z: [M+H]⁺ Calcd for C₁₉H₃₅N₅₀₆S 462.2; Found 462.2.

### Experimental Procedure for 7-(prop-2-yn-1-yl)-2-oxa-7-azaspiro[3.5]nonane (42)

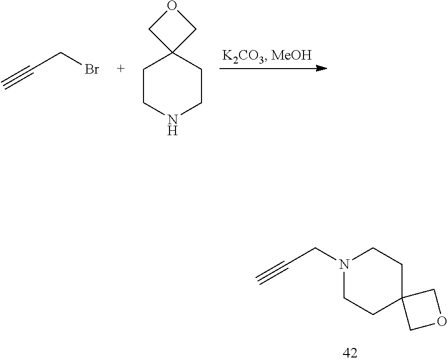

3-Bromoprop-1-yne (4.4 mL, 39.32 mmol 1.0 eq) was added to a mixture of 2-oxa-7-azaspiro[3.5]nonane (8.54 g, 39.32 mmol, 1.0 eq), potassium carbonate (17.9 g, 129.7 mmol, 3.3 eq) in Methanol (200 mL) and stirred over night at room temperature. The mixture was filtered, Celite was added and the solvent was removed under reduced pressure. The residue was purified by silica gel chromatography (220 g) using dichloromethane/methanol as mobile phase. The concentration of methanol was gradually increased from 0% to 5% to afford 7-(prop-2-yn-1-yl)-2-oxa-7-azaspiro[3.5]nonane (42, 4.44 g, 68%) as an oil.

### Experimental Procedure for 2-(2-(2-(2-(4-((2-oxa-7-azaspiro[3.5]nonan-7-yl) methyl)-1H-1,2,3-triazol-1-yl)ethoxy)ethoxy)ethoxy)ethan-1-amine (43)

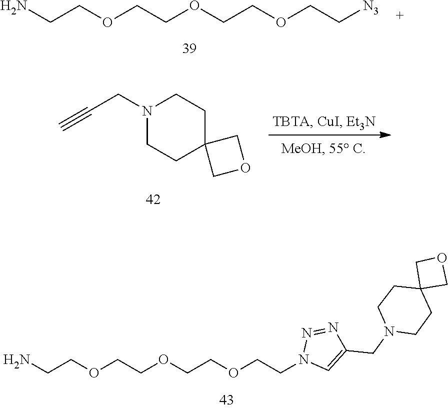

A mixture of 7-(prop-2-yn-1-yl)-2-oxa-7-azaspiro[3.5]nonane (42, 2.5 g, 15.13 mmol, 1.0 eq), Tris[(1-benzyl-1H-1,2,3-triazol-4-yl)methyl]-amine (1.77 g, 3.33 mmol, 0.22 eq), Copper Iodide (288 mg, 1.51 mmol, 0.1 eq), and Triethylamine (0.53 mL, 3.8 mmol, 0.25 eq) in Methanol (50 mL) was cooled with an ice bath. 2-(2-(2-(2-azidoethoxy)ethoxy)ethoxy)ethan-1-amine (39, 3.86 g, 17.70 mmol, 1.17 eq) was added in a dropwise fashion, the cooling bath was removed and the mixture was stirred for 5 minutes. The reaction was warmed to 55° C. and stirred over night under Nitrogen atmosphere. The reaction mixture was cooled to room temperature, Celite (10 g) was added, and concentrated under reduced pressure. The crude product was purified over silica gel (220 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 10% to afford for 2-(2-(2-(2-(4-((2-oxa-7-azaspiro[3.5]nonan-7-yl) methyl)-1H-1,2,3-triazol-1-yl)ethoxy)ethoxy)ethoxy)ethan-1-amine (43, 4.76 g, 82%) as an oil. LCMS m/z: [M+H]⁺ Calcd for C₁₈H₃₃N₅O₄ 384.3; Found 384.2.

### Experimental Procedure for N-(2-(2-(2-(2-(4-((2-oxa-7-azaspiro[3.5]nonan-7-yl)methyl)-1H-1,2,3-triazol-1-yl)ethoxy)ethoxy)ethoxy)ethyl)methacrylamide (44)

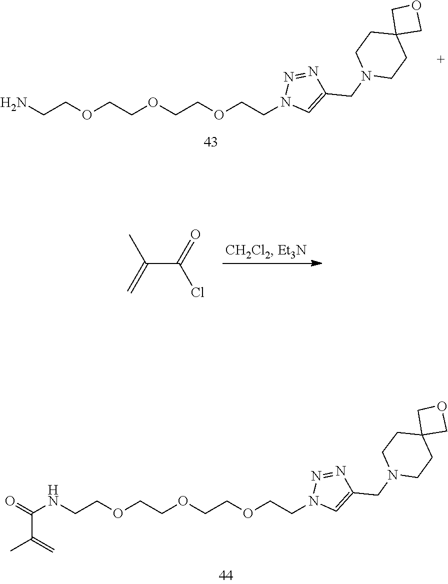

A solution of 2-(2-(2-(2-(4-((2-oxa-7-azaspiro[3.5]nonan-7-yl) methyl)-1H-1,2,3-triazol-1-yl)ethoxy)ethoxy)ethoxy)ethan-1-amine (43, 2.65 g, 6.91 mmol, 1.0 eq) and triethylamine (1.16 mL, 8.29 mmol, 1.2 eq) in CH₂Cl₂ (100 mL) was cooled with an ice-bath under Nitrogen atmosphere. Methacryloyl chloride (0.74 mL, 7.6 mmol, 1.1 eq) was added in a dropwise fashion. The cooling bath was removed and the reaction mixture was stirred for 4 h at room temperature. 10 grams of Celite was added and the solvent was removed under reduced pressure. The residue was purified by silica gel chromatography (120 g) using dichloromethane/methanol as mobile phase. The concentration of methanol was gradually increased from 0% to 10% to afford N-(2-(2-(2-(2-(4-((2-oxa-7-azaspiro[3.5]nonan-7-yl)methyl)-1H-1,2,3-triazol-1-yl)ethoxy)ethoxy)ethoxy)ethyl)methacrylamide (44, 1.50 g, 48% yield) as a colorless oil. LCMS m/z: [M+H]⁺ Calcd for C₂₂H₃₇N₅O₅ 452.29; Found 452.25.

### Experimental Procedure for 4-((1-(2-(2-aminoethoxy)ethyl)-1H-1,2,3-triazol-4-yl)methyl)thiomorpholine 1,1-dioxide (45)

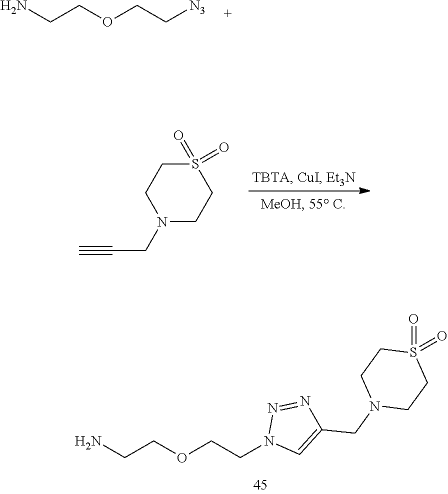

A mixture of 4-(prop-2-yn-1-yl)thiomorpholine 1,1-dioxide (1.14 g, 6.58 mmol, 1.0 eq), Tris[(1-benzyl-1H-1,2,3-triazol-4-yl)methyl]-amine (768 mg, 1.45 mmol, 0.22 eq), Copper Iodide (125 mg, 0.66 mmol, 0.1 eq), and Triethylamine (0.23 mL, 1.65 mmol, 0.25 eq) in Methanol (20 mL) was cooled with an ice bath. 2-(2-azidoethoxy)ethan-1-amine (1.00 g, 7.70 mmol, 1.17 eq) was added in a dropwise fashion, the cooling bath was removed and the mixture was stirred for 5 minutes. The reaction was warmed to 55° C. and stirred over night under Nitrogen atmosphere. The reaction mixture was cooled to room temperature, Celite (10 g) was added, and concentrated under reduced pressure. The crude product was purified over silica gel (40 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 9.5% to afford for 4-((1-(2-(2-aminoethoxy)ethyl)-1H-1,2,3-triazol-4-yl)methyl)thiomorpholine 1,1-dioxide (45, 1.86 g, 93%) as a white solid. LCMS m/z: [M+H]⁺ Calcd for C₁₁H₂₁N₅O₄S 304.1438; Found 304.1445.

### Experimental Procedure for N-(2-(2-(4-((1,1-dioxidothiomorpholino)methyl)-1H-1,2,3-triazol-1-yl)ethoxy)ethyl)methacrylamide (46)

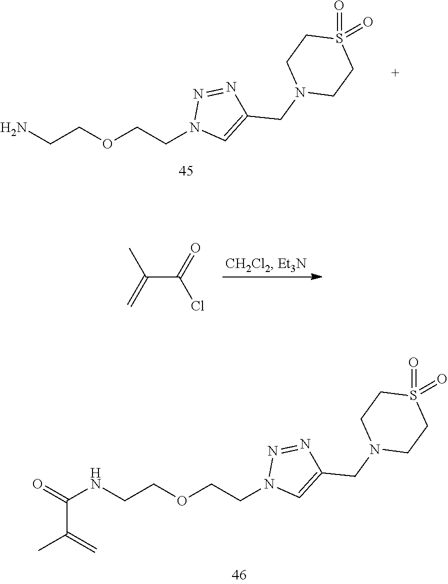

A solution of 4-((1-(2-(2-aminoethoxy)ethyl)-1H-1,2,3-triazol-4-yl)methyl)thiomorpholine 1,1-dioxide (45, 1.32 g, 4.35 mmol, 1.0 eq) and triethylamine (0.73 mL, 5.22 mmol, 1.2 eq) in CH₂Cl₂ (100 mL) was cooled with an ice-bath under Nitrogen atmosphere. Methacryloyl chloride (0.47 mL, 4.8 mmol, 1.1 eq) was added in a dropwise fashion. The cooling bath was removed and the reaction mixture was stirred for 4 h at room temperature. 10 grams of Celite was added and the solvent was removed under reduced pressure. The residue was purified by silica gel chromatography (120 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 1.25% to afford N-(2-(2-(4-((1,1-dioxidothiomorpholino)methyl)-1H-1,2,3-triazol-1-yl)ethoxy)ethyl)-methacrylamide (46, 0.90 g, 56% yield) as a colorless oil. LCMS m/z: [M+H]⁺ Calcd for C₁₅H₂₅N₅O₄S 372.17; Found 372.15.

### Experimental Procedure for 4-((1-(2-(2-(2-aminoethoxy)ethoxy)ethyl)-1H-1,2,3-triazol-4-yl)methyl)thiomorpholine 1,1-dioxide (47)

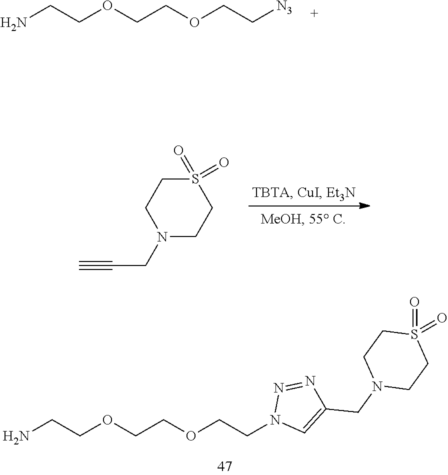

A mixture of 4-(prop-2-yn-1-yl)thiomorpholine 1,1-dioxide (4.6 g, 26.55 mmol, 1.0 eq), Tris[(1-benzyl-1H-1,2,3-triazol-4-yl)methyl]-amine (3.1 g, 5.84 mmol, 0.22 eq), Copper Iodide (506 mg, 2.66 mmol, 0.1 eq), and Triethylamine (0.93 mL, 6.64 mmol, 0.25 eq) in Methanol (80 mL) was cooled with an ice bath. 2-(2-(2-azidoethoxy)ethoxy)ethan-1-amine (5.00 g, 28.68 mmol, 1.08 eq) was added in a dropwise fashion, the cooling bath was removed and the mixture was stirred for 5 minutes. The reaction was warmed to 55° C. and stirred over night under Nitrogen atmosphere. The reaction mixture was cooled to room temperature, Celite was added, and concentrated under reduced pressure. The crude product was purified over silica gel (220 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 10% to afford for 4-((1-(2-(2-(2-aminoethoxy)ethoxy)ethyl)-1H-1,2,3-triazol-4-yl)methyl)thiomorpholine 1,1-dioxide (47, 5.26 g, 57%) as a yellowish oil. LCMS m/z: [M+H]⁺ Calcd for C₁₃H₂₅N₅O₄S 348.1700; Found 348.1700.

### Experimental Procedure N-(2-(2-(2-(4-((1,1-dioxidothiomorpholino)methyl)-1H-1,2,3-triazol-1-yl)ethoxy)ethoxy)ethyl)methacrylamide (48)

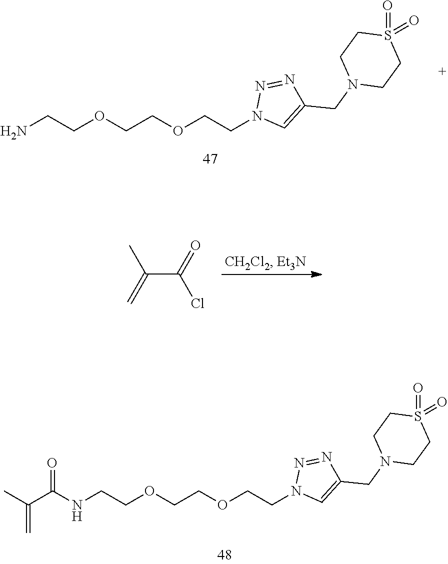

A solution of 4-((1-(2-(2-(2-aminoethoxy)ethoxy)ethyl)-1H-1,2,3-triazol-4-yl)methyl)thiomorpholine 1,1-dioxide (47, 1.49 g, 4.29 mmol, 1.0 eq) and triethylamine (0.72 mL, 5.15 mmol, 1.2 eq) in CH₂Cl₂ (50 mL) was cooled with an ice-bath under Nitrogen atmosphere. Methacryloyl chloride (0.46 mL, 4.7 mmol, 1.1 eq) was added in a dropwise fashion. The cooling bath was removed and the reaction mixture was stirred for 4 h at room temperature. 10 grams of Celite was added and the solvent was removed under reduced pressure. The residue was purified by silica gel chromatography (80 g) using dichloromethane/methanol as mobile phase. The concentration of methanol was gradually increased from 0% to 5% to afford N-(2-(2-(2-(4-((1,1-dioxidothiomorpholino)methyl)-1H-1,2,3-triazol-1-yl)ethoxy)ethoxy)ethyl)-methacrylamide (48, 0.67 g, 38% yield) as a colorless oil. LCMS m/z: [M+H]⁺ Calcd for C₁₇H₂₉N₅O₅S 416.20; Found 416.20.

### Experimental Procedure for 4-((1-(14-amino-3,6,9,12-tetraoxatetradecyl)-1H-1,2,3-triazol-4-yl)methyl)thiomorpholine 1,1-dioxide (49)

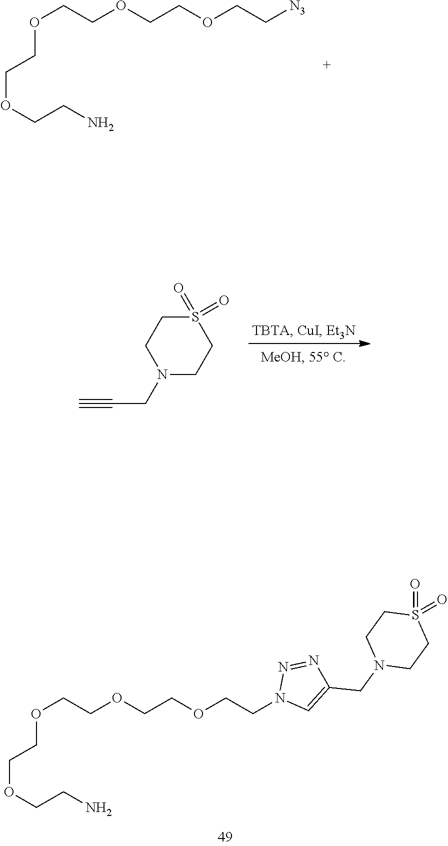 [SMILES: [N-]=[N+]=NCCOCCOCCOCCOCCN ; "+" ; C#CCN1CCS(=O)(=O)CC1 ; NCCOCCOCCOCCOCCn1cc(CN2CCS(=O)(=O)CC2)nn1]

A mixture of 4-(prop-2-yn-1-yl)thiomorpholine 1,1-dioxide (5.0 g, 28.86 mmol, 1.0 eq), Tris[(1-benzyl-1H-1,2,3-triazol-4-yl)methyl]-amine (3.37 g, 6.35 mmol, 0.22 eq), Copper Iodide (550 mg, 2.89 mmol, 0.1 eq), and Triethylamine (1.01 mL, 7.22 mmol, 0.25 eq) in Methanol (90 mL) was cooled with an ice bath. 14-azido-3,6,9,12-tetraoxatetradecan-1-amine (8.86 g, 33.77 mmol, 1.17 eq) was added in a dropwise fashion, the cooling bath was removed and the mixture was stirred for 5 minutes. The reaction was warmed to 55° C. and stirred over night under Nitrogen atmosphere. The reaction mixture was cooled to room temperature, Celite (15 g) was added, and concentrated under reduced pressure. The crude product was purified over silica gel (220 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 10% to afford for 4-((1-(14-amino-3,6,9,12-tetraoxatetradecyl)-1H-1,2,3-triazol-4-yl)methyl)thiomorpholine 1,1-dioxide (49, 7.56 g, 60%) as an oil. LCMS m/z: [M+H]⁺ Calcd for C₁₇H₃₃N₅O₆S 436.2224; Found 436.2228.

### Experimental Procedure N-(14-(4-((1,1-dioxidothiomorpholino)methyl)-1H-1,2,3-triazol-1-yl)-3,6,9,12-tetraoxatetradecyl)methacrylamide (50)

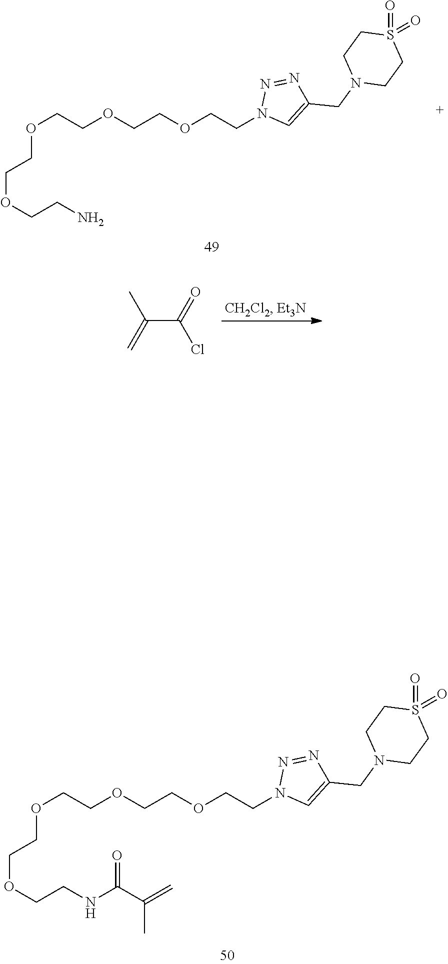

A solution of 4-((1-(14-amino-3,6,9,12-tetraoxatetradecyl)-1H-1,2,3-triazol-4-yl)methyl)thiomorpholine 1,1-dioxide (49, 1.95 g, 4.79 mmol, 1.0 eq) and triethylamine (0.80 mL, 5.74 mmol, 1.2 eq) in CH₂Cl₂ (50 mL) was cooled with an ice-bath under Nitrogen atmosphere. Methacryloyl chloride (0.51 mL, 5.26 mmol, 1.1 eq) was added in a dropwise fashion. The cooling bath was removed and the reaction mixture was stirred for 4 h at room temperature. 10 grams of Celite was added and the solvent was removed under reduced pressure.

The residue was purified by silica gel chromatography (80 g) using dichloromethane/methanol as mobile phase. The concentration of methanol was gradually increased from 0% to 5% to afford N-(14-(4-((1,1-dioxidothiomorpholino)methyl)-1H-1,2,3-triazol-1-yl)-3,6,9,12-tetraoxatetradecyl)methacrylamide (50, 0.76 g, 32% yield) as a colorless oil. LCMS m/z: [M+H]⁺ Calcd for C₂₁H₃₇N₅O₇S 504.25; Found 504.20.

### Example 4: Chemical Modification of Alginate for Cell Encapsulation

A polymeric material may be chemically modified with a compound of Formula (I) (or a pharmaceutically acceptable salt thereof) prior to encapsulation of MSFCs (e.g., MSCs) to form an implantable element. Synthetic protocols of exemplary compounds for modification of polymeric materials are outlined above in Example 3. These compounds, or others, may be used to chemically modify any polymeric material. For example, in the case of alginate, the alginate carboxylic acid is activated for coupling to one or more amine-functionalized compounds to achieve an alginate modified with a compound, e.g., a compound of Formula (I). The alginate polymer is dissolved in water (30 mL/gram polymer) and treated with 2-chloro-4,6-dimethoxy-1,3,5-triazine (0.5 eq) and N-methylmorpholine (1 eq). To this mixture is added a solution of the compound of interest in acetonitrile (0.3M). The reaction is warmed to 55° C. for 16 h, then cooled to room temperature and gently concentrated via rotary evaporation, then the residue is dissolved in water. The mixture is filtered through a bed of cyano-modified silica gel (Silicycle) and the filter cake was washed with water. The resulting solution is then dialyzed (10,000 MWCO membrane) against water for 24 hours, replacing the water twice. The resulting solution is concentrated via lyophilization to afford the desired chemically modified alginate.

### Example 5: Formation of In Situ Encapsulated Implantable Elements

The MSFC (e.g., human MSC) clusters were encapsulated in alginate to form in-situ encapsulated implantable elements according to the protocol described herein. To solubilize alginate, SLG20 (NovaMatrix, Sandvika, Norway, cat. #4202006) was dissolved at 1.4% weight to volume in 0.8% saline. Modified alginate (e.g., as described in Example 4) was initially dissolved at 5% weight to volume in 0.8% saline and then blended with 3% weight to volume SLG100 (also dissolved in 0.8% saline) at a volume ratio of 80% TMTD alginate to 20% SLG100.

Prior to fabrication of the in-situ encapsulated implantable elements, buffers were sterilized by autoclaving, and alginate solutions were sterilized by filtration through a 0.2-μm filter using aseptic processes. An electrostatic droplet generator was set up as follows: an ES series 0-100-kV, 20-watt high-voltage power generator (Gamma ES series, Gamma High-Voltage Research, FL, USA) was connected to the top and bottom of a blunt-tipped needle (SAI Infusion Technologies, IL, USA). This needle was attached to a 5-ml Luer-lock syringe (BD, NJ, USA), which was clipped to a syringe pump (Pump 11 Pico Plus, Harvard Apparatus, MA, USA) that was oriented vertically. The syringe pump pumps alginate out into a glass dish containing a 20 mM barium 5% mannitol solution (Sigma-Aldrich, MO, USA). The settings of the PicoPlus syringe pump were 12.06 mm diameter and 0.2 ml/min flow rate.

In-situ encapsulated implantable elements (0.5-mm sphere size) were generated with a 25G blunt needle, a voltage of 5 kV and a 200 μl/min flow rate. For formation of 1.5-mm spheres (e.g., capsules), an 18-gauge blunt-tipped needle (SAI Infusion Technologies) was used with a flow-rate of 0.16 mL/min or 10 mL/hr and adjusting the voltage in a range of 5-9 kV until there are 12 drops per 10 seconds. Immediately before encapsulation, the cultured MSFC clusters were centrifuged at 1,400 r.p.m. for 1 min and washed with calcium-free Krebs-Henseleit (KH) Buffer (4.7 mM KCl, 25 mM HEPES, 1.2 mM KH2PO4, 1.2 mM MgSO4×7H2O, 135 mM NaCl, pH≈7.4, ≈290 mOsm). After washing, the cells were centrifuged again and all of the supernatant was aspirated. The cell pellet was then resuspended in the alginate solutions (described above) at a range of cluster densities of clusters per ml alginate solution. The in-situ encapsulated implantable elements were crosslinked using a BaCl₂ gelling solution, and their sizes were controlled as described above. Immediately after cross-linking, the in-situ encapsulated implantable elements were washed with HEPES buffer (NaCl 15.428 g, KCl 0.70 g, MgCl₂.6H₂O 0.488 g, 50 ml of HEPES (1 M) buffer solution (Gibco, Life Technologies, California, USA) in 2 liters of deionized water) four times, and stored at 4° C. until use. After formation and prior to use, the in-situ encapsulated implantable elements were analyzed by light microscopy to determine size and assess capsule quality. The final number of cell clusters of encapsulated capsules was counted after encapsulation.

### Example 6: Transfection of Human MSCs

Human MSC cells from ATCC (Bone Marrow-Derived Mesenchymal Stem Cells; Normal, Human (ATCC PCS-500-012™)) were transfected with a vector encoding Gla, IDUA or SGSH using standard transfection techniques. Two days after transfection, the cell line was cultured for about 2 weeks in the presence of puromycin.

### Example 7: Secretion of Factor VIII-BDD from In Situ Encapsulated Implantable Elements

Human MSC cells are transfected with a vector encoding human Factor VIII-BDD using standard transfection techniques. Two days after transfection, the cell line is cultured for about 2 weeks in the presence of puromycin, and the cells are then encapsulated in 1.5 mm alginate implantable elements as outlined in Example 5.

In order to determine the amount of Factor VIII-BDD available, the encapsulated cells (Cap) are spun down and the supernatant is collected and analyzed for the presence of human Factor VIII-BDD at 4 hours, 24 hours, 48 hours, and 72 hours after transfection. These results are compared with unencapsulated MSCs.

The implantable elements are further examined by microscopy to assess cell viability and capsule integrity.

### Example 8: Evaluation of Encapsulated Implantable Elements In Vivo

Encapsulated implantable elements comprising engineered MSFCs (e.g., engineered MSC cells) may be evaluated in mice according to the procedure below.

Preparation: Mice are prepared for surgery by being placed under anesthesia under a continuous flow of 1-4% isofluorane with oxygen at 0.5 L/min. Preoperatively, all mice receive a 0.05-0.1 mg/kg of body weight dose of buprenorphine subcutaneously as a pre-surgical analgesic, along with 0.5 ml of 0.9% saline subcutaneously to prevent dehydration. A shaver with size #40 clipper blade is used to remove hair to reveal an area of about 2 cm×2 cm on ventral midline of the animal abdomen. The entire shaved area is aseptically prepared with a minimum of 3 cycles of scrubbing with povidine (in an outward centrifugal direction from the center of the incision site when possible), followed by rinsing with 70% alcohol. A final skin paint with povidine is also applied. The surgical site is draped with sterile disposable paper to exclude surrounding hair from touching the surgical site, after disinfection of table top surface with 70% ethanol. Personnel use proper PPE, gowning and surgical gloves.

Surgical procedure: A sharp surgical blade or scissor is used to cut a 0.5-0.75 cm midline incision through the skin and the linea alba into the abdomen of the subject mice. The incision should be as small as possible with 0.75 cm being the largest possible incision size. A sterile plastic pipette is used to transfer the alginate microcapsules (with or without cells) into the peritoneal cavity. The abdominal muscle is closed by suturing with 5-0 Ethicon black silk or PDS-absorbable 5.0-6.0 monofilament absorbable thread, and the external skin layer is closed using wound clips. These wound clips are removed 7-10 d post-surgery after complete healing is confirmed. Blood and tissue debris is removed from the surgical instruments between procedures and the instruments are also re-sterilized between animals using a hot bead sterilizer. After the surgery, the animals are put back in the cage on a heat pad or under a heat lamp and monitored until they came out of anesthesia.

Intraoperative care: Animals are kept warm using Deltaphase isothermal pad. The animal's eyes are hydrated with sterile ophthalmic ointment during the period of surgery. Care is taken to avoid wetting the surgical site excessively to avoid hypothermia. Respiratory rate and character are monitored continuously. If vital signs are indicative of extreme pain and distress, the animal is euthanized via cervical dislocation.

At the desired time-point post-operation, the animal is euthanized by CO₂ asphyxiation and the alginate capsules are collected by peritoneal lavage.

Exemplary mouse strains useful in these experiments include AKXL37/TyJ; Factor IX deficient strain B6.129P2-F9^{tm1Dws/}J; a Factor VIII deficient strain described in Bi, L et al (1995) Nature 10:119-121); alpha-galactosidase stain B6;129-Gla^{tm1Kul}/J described in Ohshima, T et al. (1997) Proc Nat'l Sci USA 94:2540-2544); and the Factor IX deficient stain described in Lin, H-F et al. (2017) Blood 90: 3962-3966.

### Example 9: Comparison of Encapsulation Architecture of Engineered MSFCs

A study comparing encapsulation of single engineered MSFCs (e.g., single MSC cells or single MSC cell derivatives), clusters of engineered MSFCs (e.g., clusters of engineered MSC cells or clusters of MSC cell derivatives), and engineered MSFCs bound to a microcarrier (e.g., engineered MSC cells bound to a microcarrier), or in 1.5 mm alginate implantable elements, is conducted to gauge production of a therapeutic agent (e.g., a protein) and cell viability. The maximum cell loading is determined for each architecture, and comparisons across architectures is made at equal cell loading and at maximal cell loading for each architecture. Cell loading, viability, morphology and protein secretion is assessed in vitro and in vivo. For in vivo pharmacokinetics analysis, capsules are implanted IP into mice according to the protocol outlined in Example 8, and at specified time points, protein is detected in the blood via ELISA, and capsules are explanted to determine the cell viability.

### ENUMERATED EMBODIMENTS

1. An implantable element comprising a plurality of engineered MSFCs (e.g., engineered MSCs) that produces or releases a therapeutic agent (e.g., a nucleic acid (e.g., a nucleotide, DNA, or RNA), a polypeptide, a lipid, a sugar (e.g., a monosaccharide, disaccharide, oligosaccharide, or polysaccharide), or a small molecule), wherein:
a) the plurality of engineered MSFCs (e.g., engineered MSCs) or the implantable element produces or releases the therapeutic agent for at least 5 days, at least 10 days, at least one month, or at least 3 months, e.g., when implanted into a subject (e.g., a human subject) or when evaluated by a reference method described herein, e.g., polymerase chain reaction or in situ hybridization for nucleic acids; mass spectroscopy for lipid, sugar and small molecules; microscopy and other imaging techniques for agents modified with a fluorescent or luminescent tag, and ELISA or Western blotting for polypeptides;
b) the plurality of engineered MSFCs (e.g., engineered MSCs) or the implantable element produces or releases at least 10 picograms of the therapeutic agent per day, e.g., produces at least 10 picograms of the therapeutic agent per day for at least 5 days, e.g., when cultured in vitro, when implanted into a subject (e.g., a human subject), or when evaluated by a reference method, e.g., an applicable reference method listed in part a) above;
c) the plurality of engineered MSFCs (e.g., engineered MSCs) or the implantable element produces or releases the therapeutic agent at a rate, e.g., of at least 10 picograms of therapeutic agent per day, which is at least 50% (e.g., at least 60%, at least 70%, at least 80%, at least 90%, at least 95%, or at least 99%) of the rate control cells produce when, e.g., not encapsulated in the implantable element or not embedded or implanted in a subject (e.g., a human subject), e.g., an applicable reference method listed in part a) above;
d) the plurality of engineered MSFCs (e.g., engineered MSCs) or the implantable element produces or releases the therapeutic agent for at least 5 days and the amount released per day does not vary more than 50% (e.g., at least about 40%, about 30%, about 20%, about 10%, about 5%, or less), e.g. an applicable reference method listed in part a) above;
e) upon introduction into a subject (e.g., a human subject), sufficient therapeutic agent is produced or released by the plurality of engineered MSFCs or the implantable element such that a location at least about 5 cm, about 10 cm, about 25 cm, about 50 cm, about 100 cm, or 150 cm away from the introduced element receives an effective concentration (e.g., a therapeutically effective concentration) of the therapeutic agent (e.g., a therapeutically effective concentration found in the pancreas, liver, blood, or outside the eye), e.g., as evaluated by an applicable reference method listed in part a) above;
f) sufficient therapeutic agent is produced or released by the plurality of engineered MSFCs or the implantable element such that when embedded or implanted in the peritoneal cavity of a subject (e.g., a human subject), e.g., a detectable level of the therapeutic agent, e.g., 10 picograms, is found at a location at least 5 cm, 10 cm, 25 cm, 50 cm, 100 cm, or 150 cm away from the engineered MSFC (e.g., an engineered MSC), e.g., as evaluated by an applicable reference method listed in part a) above;
g) upon introduction into a subject (e.g., a human subject), sufficient therapeutic agent is produced or released by the plurality of engineered MSFCs or the implantable element such that about 50% of the therapeutic agent produced or released (about 60%, about 70%, about 80%, about 90%, or about 99% of the therapeutic agent produced or released) enters the circulation (e.g., peripheral circulation) of a subject, e.g., as evaluated by an applicable reference method listed in part a) above;
h) the plurality of engineered MSFCs (e.g., engineered MSCs) is capable of phagocytosis, e.g., is capable of about 99%, about 95%, about 90%, about 85%, about 80%, about 75%, about 70%, about 60%, or about 50% of the level of phagocytosis compared with reference non-engineered MSFCs (e.g., non-engineered MSCs), e.g., as evaluated by fluorescein-labeled antibody assay, microscopy (e.g., fluorescence microscopy (e.g., time-lapse or evaluation of spindle formation), or flow cytometry;
i) the plurality of engineered MSFCs (e.g., engineered MSCs) is capable of autophagy, e.g., is capable of about 99%, about 95%, about 90%, about 85%, about 80%, about 75%, about 70%, about 60%, or about 50% of the level of autophagy compared with reference non-engineered MSFCs (e.g., non-engineered MSCs), e.g., as evaluated by 5-ethynyl-2′deoxyuridine (EdU) assay, 5-bromo-2′-deoxyuridine (BrdU) assay, cationic amphiphilic tracer (CAT) assay, or microscopy (e.g., fluorescence microscopy (e.g., time-lapse or evaluation of spindle formation), immunoblotting analysis of LC3 and p62, detection of autophagosome formation by fluorescence microscopy, and monitoring autophagosome maturation by tandem mRFP-GFP fluorescence microscopy;
j) the plurality of engineered MSFCs (e.g., engineered MSCs) has a form factor described herein, e.g., as a cluster, spheroid, or aggregate of engineered MSFCs (e.g., an engineered MSCs);
k) the plurality of engineered MSFCs (e.g., engineered MSCs) is disposed on a non-cellular carrier (e.g, a microcarrier, e.g., a bead, e.g., a polyester, polystyrene, or polymeric bead);
l) the plurality of engineered MSFCs (e.g., engineered MSCs) proliferates or is capable of proliferating after encapsulation in the implantable element, e.g., as determined by microscopy;
m) the plurality of engineered MSFCs (e.g., engineered MSCs) does not proliferate or is not capable of proliferating after encapsulation in the implantable element, e.g., as determined by microscopy (e.g., 5-ethynyl-2′deoxyuridine (EdU) assay); or
n) upon introduction, administration, or implantation into a subject (e.g., a human subject), sufficient therapeutic agent is produced or released by the plurality of engineered MSFCs (e.g., engineered MSCs) or the implantable element such that an effective concentration (e.g., a therapeutically effective concentration) of the therapeutic agent is found in the peripheral bloodstream (e.g., a therapeutically effective concentration found in the pancreas, liver, blood, or outside the eye).
   2. An implantable element comprising a plurality of engineered MSFCs (e.g., engineered MSCs) comprising an exogenous nucleic acid which promotes and/or conditions the production of a polypeptide, e.g., a therapeutic polypeptide, wherein the plurality of engineered MSFCs (e.g., engineered MSCs) produces or releases the polypeptide for at least 5 days, e.g., when implanted into a subject (e.g., a human subject) or when evaluated by a reference method, e.g., ELISA or Western blotting
   3. An implantable element comprising a plurality of engineered MSFCs (e.g., engineered MSCs) comprising an exogenous nucleic acid which promotes and/or conditions the production of a polypeptide, e.g., a therapeutic polypeptide, wherein the plurality of engineered MSFCs (e.g., engineered MSCs) produces or releases at least 10 picograms of the polypeptide per day, e.g., produces at least 10 picograms of the polypeptide per day for at least 5 days, e.g., when implanted into a subject (e.g., a human subject) or when evaluated by a reference method, e.g., ELISA or Western blotting.
   4. An implantable element comprising a plurality of engineered MSFCs (e.g., engineered MSCs) comprising an exogenous nucleic acid which promotes and/or conditions the production of a polypeptide, e.g., a therapeutic polypeptide, wherein the plurality of engineered MSFCs (e.g., engineered MSCs) produces or releases the polypeptide at a rate, e.g., of at least 10 picograms of polypeptide per day, which is at least 50% (e.g., at least 60%, at least 70%, at least 80%, at least 90%, at least 95%, or at least 99%) of the rate of reference cells not encapsulated in the implantable element or not embedded or implanted in a subject (e.g., a human subject), e.g., as evaluated by ELISA or Western blotting.
   5. An implantable element comprising a plurality of engineered MSFCs (e.g., engineered MSCs) comprising an exogenous nucleic acid which promotes and/or conditions the production of a polypeptide, e.g., a therapeutic polypeptide, wherein the plurality of engineered MSFCs (e.g., engineered MSCs) produces or releases the polypeptide for at least 5 days (e.g., after implantation into a subject, e.g., a human subject) and the amount released per day does not vary more than 50% (e.g., at least about 40%, about 30%, about 20%, about 10%, about 5%, or less), e.g. as evaluated by ELISA or Western blotting.
   6. An implantable element comprising a plurality engineered MSFCs (e.g., engineered MSCs) comprising an exogenous nucleic acid which promotes and/or conditions the production of a polypeptide, e.g., a therapeutic polypeptide, wherein upon introduction into a subject (e.g., a human subject), sufficient polypeptide is produced or released such that a location at least about 5 cm, about 10 cm, about 25 cm, about 50 cm, or about 100 cm away receives an effective concentration (e.g., a therapeutically effective concentration) of the polypeptide (e.g., a therapeutically effective concentration found in the pancreas, liver, blood, or outside the eye).
   7. An implantable element comprising a plurality engineered MSFCs (e.g., engineered MSCs) comprising an exogenous nucleic acid which promotes and/or conditions the production of a polypeptide, e.g., a therapeutic polypeptide, wherein sufficient polypeptide is produced or released such that when embedded or implanted in the peritoneal cavity of a subject (e.g., a human subject), e.g., a detectable level of the polypeptide, e.g., 10 picograms, is found at a location at least 5 cm, 10 cm, 25 cm, 50 cm, 100 cmm or 150 cm away from the element.
   8. An implantable element comprising a plurality of engineered MSFCs (e.g., engineered MSCs) comprising an exogenous nucleic acid which promotes and/or conditions the production of a polypeptide, e.g., a therapeutic polypeptide, wherein upon introduction into a subject (e.g., a human subject), sufficient polypeptide is produced or released such that about 50% of the polypeptide produced or released (about 60%, about 70%, about 80%, about 90%, or about 99% of the therapeutic polypeptide produced or released) enters the circulation (e.g., peripheral circulation) of the subject.
   9. An implantable element comprising a plurality of engineered MSFCs (e.g., engineered MSCs) comprising an exogenous nucleic acid which promotes and/or conditions the production of a polypeptide, e.g., a therapeutic polypeptide, wherein the engineered MSFCs (e.g., engineered MSCs) is capable of phagocytosis, e.g., is capable of about 99%, about 95%, about 90%, about 85%, about 80%, about 75%, about 70%, about 60%, or about 50% of the level of phagocytosis compared with reference non-engineered MSFCs (e.g., non-engineered MSCs), e.g., as evaluated by fluorescein-labeled antibody assay, microscopy (e.g., fluorescence microscopy (e.g., time-lapse or evaluation of spindle formation), or flow cytometry.
   10. An implantable element comprising a plurality of engineered MSFCs (e.g., engineered MSCs) comprising an exogenous nucleic acid which promotes and/or conditions the production of a polypeptide, e.g., a therapeutic polypeptide, wherein the plurality of engineered MSFCs (e.g., engineered MSCs) is capable of autophagy, e.g., is capable of about 99%, about 95%, about 90%, about 85%, about 80%, about 75%, about 70%, about 60%, or about 50% of the level of autophagy compared with reference non-engineered MSFCs (e.g., non-engineered MSCs), e.g., as evaluated by 5-ethynyl-2′deoxyuridine (EdU) assay, 5-bromo-2′-deoxyuridine (BrdU) assay, cationic amphiphilic tracer (CAT) assay, or microscopy (e.g., fluorescence microscopy (e.g., time-lapse or evaluation of spindle formation), immunoblotting analysis of LC3 and p62, detection of autophagosome formation by fluorescence microscopy, and monitoring autophagosome maturation by tandem mRFP-GFP fluorescence microscopy.
   11. An implantable element comprising a plurality of engineered MSFCs (e.g., engineered MSCs) comprising an exogenous nucleic acid which promotes and/or conditions the production of a polypeptide, e.g., a therapeutic polypeptide, wherein the plurality of engineered MSFCs (e.g., engineered MSCs) is provided having a form factor described herein, e.g., as a cluster, spheroid, or aggregate of engineered MSFCs (e.g., engineered MSCs).
   12. An implantable element comprising a plurality of engineered MSFCs (e.g., engineered MSCs) comprising an exogenous nucleic acid which promotes and/or conditions the production of a polypeptide, e.g., a therapeutic polypeptide, wherein the plurality of engineered MSFCs (e.g., engineered MSCs) is disposed on a non-cellular carrier (e.g, a microcarrier, e.g., a bead, e.g., a polyester, polystyrene, or polymeric bead).
   13. An implantable element comprising a plurality of engineered MSFCs (e.g., engineered MSCs) comprising an exogenous nucleic acid which promotes and/or conditions the production of a polypeptide, e.g., a therapeutic polypeptide, wherein the plurality of engineered MSFCs (e.g., engineered MSCs) proliferates or is capable of proliferating after encapsulation in the implantable element, e.g., as determined by microscopy.
   14. An implantable element comprising a plurality of engineered MSFCs (e.g., engineered MSCs) comprising an exogenous nucleic acid which promotes and/or conditions the production of a polypeptide, e.g., a therapeutic polypeptide, wherein the plurality of engineered MSFCs (e.g., engineered MSCs) does not proliferate or is not capable of proliferating after encapsulation in the implantable element, e.g., as determined by microscopy.
   15. An implantable element comprising a plurality of engineered MSFCs (e.g., engineered MSCs) comprising an exogenous nucleic acid which promotes and/or conditions the production of a polypeptide, e.g., a therapeutic polypeptide, wherein upon introduction, administration, or implantation into a subject (e.g., a human subject), sufficient polypeptide is produced or released such that an effective concentration (e.g., a therapeutically effective concentration) of the polypeptide is found in the peripheral bloodstream of the subject (e.g., a therapeutically effective concentration found in the pancreas, liver, blood, or outside the eye).
   16. An implantable element comprising a plurality of engineered MSFCs (e.g., engineered MSCs) that produces or releases a therapeutic agent (e.g., a nucleic acid (e.g., a nucleotide, DNA, or RNA), a polypeptide, a lipid, a sugar (e.g., a monosaccharide, disaccharide, oligosaccharide, or polysaccharide), or a small molecule).
   17. Any of embodiments 2 to 16, wherein the exogenous nucleic acid is an RNA (e.g., an mRNA) molecule or a DNA molecule.
   18. Any of embodiments 1 to 17, wherein the polypeptide or therapeutic agent is selected from the group consisting of Factor I, Factor II, Factor V, Factor VII, Factor VIII, Factor IX, Factor X, Factor XI and Factor XIII polypeptides.
   19. The implantable element of any of embodiments 1 to 18, wherein the polypeptide or therapeutic agent is an insulin polypeptide (e.g., insulin A-chain, insulin B-chain, or proinsulin).
   20. The implantable element of any of embodiments 1 to 17, wherein the polypeptide or therapeutic agent is not an insulin polypeptide (e.g., not any of insulin A-chain, insulin B-chain, or proinsulin).
   21. An implantable element comprising a plurality of engineered MSFCs, each cell in the plurality comprising an exogenous nucleic acid encoding a Factor VIII-BDD (FVIII-BDD) amino acid sequence.
   22. The implantable element of embodiment 21, wherein the FVIII-BDD amino acid sequence is selected from the group consisting of:
a) SEQ ID NO:1;
b) SEQ ID NO:3;
c) SEQ ID NO:4;
d) SEQ ID NO:5;
e) SEQ ID NO:6;
f) SEQ ID NO:7;
g) SEQ ID NO:7 with an alanine instead of arginine at position 787 and an alanine instead of arginine at position 790;
h) a conservatively substituted variant of the sequence in (a), (b), (c), (d), (f) or (g); and
i) a sequence that has as least 95%, 96%, 97%, 98%, 99% or greater sequence identity with the sequence in (a), (b), (c), (d), (f), (g) or (h);
   23. An implantable element comprising a plurality of engineered MSFCs, each cell in the plurality comprising an exogenous nucleic acid encoding a Factor IX (FIX) amino acid sequence.
   24. The implantable element of embodiment 23, wherein the FIX amino acid sequence is SEQ ID NO:2 or a conservatively substituted variant thereof, or a sequence that has at least 95%, 96%, 97%, 98%, 99% or greater sequence identity with SEQ ID NO:2 or the conservatively substituted variant.
   25. The implantable element of embodiment 23, wherein the FIX amino acid sequence is SEQ ID NO:15 or a conservatively substituted variant thereof, or a sequence that has at least 95%, 96%, 97%, 98%, 99% or greater sequence identity with SEQ ID NO:15 or the conservatively substituted variant thereof.
   26. An engineered MSFC, or an implantable element comprising the MSFC, wherein the MSFC comprises an exogenous nucleic acid which comprises a promoter sequence operably linked to a coding sequence for a polypeptide.
   27. The engineered MSFC or implantable element of embodiment 26, wherein the polypeptide comprises, consists essentially of, or consists of, an amino acid sequence which is:
a) a FVIII-BDD amino acid sequence, e.g., a sequence selected from the group consisting of SEQ ID NO:1, SEQ ID NO:3, SEQ ID NO:4, SEQ ID NO:5, SEQ ID NO:6, SEQ ID NO:7 and SEQ ID NO:7 with an alanine instead of arginine at each of positions 787 and 790;
b) a FIX amino acid sequence, e.g., SEQ ID NO:2 or an amino acid sequence having at least 95%, 96%, 97% 98%, 99% or greater sequence identity with SEQ ID NO:2;
c) an Interleukin 2 amino acid sequence, e.g., SEQ ID NO:8 or an amino acid sequence having at least 95%, 96%, 97%, 98%, 99% or greater sequence identity with SEQ ID NO:8;
d) a parathyroid hormone amino acid sequence, e.g., SEQ ID NO:9 or an amino acid sequence having at least 95%, 96%, 97%, 98%, 99% or greater sequence identity with SEQ ID NO:9; or
e) a von Willebrand Factor amino acid sequence, e.g., SEQ ID NO: 11 or SEQ ID NO:12 or an amino acid sequence having at least 95%, 96%, 97%, 98%, 99% or greater sequence identity with SEQ ID NO: 11 or SEQ ID NO:12.
   28. The engineered MSFC or implantable element of any one of embodiments 26 or 27, wherein the polypeptide comprises, consists essentially of, or consists of SEQ ID NO:2
   29. The MSFC or implantable element of any one of embodiments 26 to 28, wherein the polypeptide further comprises SEQ ID NO:13 or SEQ ID NO:14.
   30. The implantable element or engineered cell of any one of the preceding embodiments, which is provided as a treatment for a disease.
   31. The implantable element or engineered cell of embodiment 30, wherein the disease is a blood clotting disease or a lysosomal storage disease (e.g., a hemophilia (e.g., Hemophilia A or Hemophilia B), Fabry Disease, Gaucher Disease, Pompe Disease, or MPS I).
   32. The implantable element or engineered MSFC of any one of the preceding any one of the preceding embodiments, which is provided as a prophylactic treatment.
   33. The implantable element of any one of the preceding embodiments, which is formulated for injection into a subject (e.g., intraperitoneal, intramuscular, or subcutaneous injection) or is formulated for implantation into a subject (e.g., into the peritoneal cavity, e.g., the lesser sac).
   34. The implantable element or engineered cell of any one of the preceding embodiments, which is implanted or injected into the lesser sac, into the omentum, or into the subcutaneous fat of a subject.
   35. The implantable element or engineered cell of any one of the preceding embodiments, which is administered to a first subject having less than about 50%, 40%, 30%, 25%, 20%, 15%, 10%, 5%, 2%, or 1% of the polypeptide (e.g., a blood clotting factor, e.g., Factor I, Factor II, Factor V, Factor VII, Factor VIII, Factor IX, Factor X, Factor XI, or Factor XIII) relative to a second subject (e.g., a healthy subject), e.g., as determined by a blood test.
   36. The implantable element or engineered cell of any one of the preceding embodiments, wherein the level of a biomarker (e.g., a serum biomarker) in a subject is monitored, e.g., in order to determine the level of efficacy of treatment.
   37. The implantable element of any one of the preceding embodiments, which comprises a cluster of engineered MSFCs, or a microcarrier (e.g., a bead or matrix comprising an MSFC or a plurality of engineered MSFCs).
   38. The implantable element of embodiment 37, wherein the plurality of engineered MSFCs or the microcarrier (e.g., a bead or matrix comprising a plurality of engineered MSFCs) produces a plurality of polypeptides.
   39. The implantable element of any one of the preceding embodiments, wherein the implantable element comprises an enclosing component.
   40. The implantable element of embodiment 39, wherein the enclosing component is formed in situ on or surrounding an engineered MSFC, a plurality of engineered MSFCs, or a microcarrier (e.g., a bead or matrix) comprising an MSFC or MSFCs.
   41. The implantable element of claim 40, wherein the enclosing component is preformed prior to combination with the enclosed engineered MSFC, a plurality of engineered MSFCs, or a microcarrier (e.g., a bead or matrix) comprising an MSFC or MSFCs.
   42. The implantable element of any one of embodiments 39-41, wherein the enclosing component comprises a flexible polymer (e.g., PLA, PLG, PEG, CMC, or a polysaccharide, e.g., alginate).
   43. The implantable element of any one of embodiments 40-42, wherein the enclosing component comprises an inflexible polymer or metal housing.
   44. The implantable element of any one of the preceding embodiments, which is chemically modified.
   45. The implantable element of any one of embodiments 39-44, wherein the enclosing component is chemically modified.
   46. The implantable element of any one of the preceding embodiments, wherein the implantable element or an enclosing component thereof is modified with a compound of Formula (I):

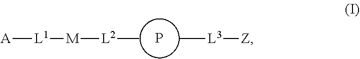

or a salt thereof, wherein:

A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₂-C₆-alkenylene)-, —N(R^{C})N(R^{D})—, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, wherein each alkyl, alkenyl, alkynyl, alkylene, alkenylene, heteroalkyl, cycloalkyl, heterocyclyl, aryl, and heteroaryl is linked to an attachment group (e.g., an attachment group defined herein) and is optionally substituted by one or more R¹;

each of L¹ and L³ is independently a bond, alkyl, or heteroalkyl, wherein each alkyl and heteroalkyl is optionally substituted by one or more R²;

L² is a bond;

M is absent, alkyl, heteroalkyl, cycloalkyl, heterocyclyl, aryl, or heteroaryl, each of which is optionally substituted by one or more R³;

P is absent, cycloalkyl, heterocycyl, or heteroaryl each of which is optionally substituted by one or more R⁴;

Z is hydrogen, alkyl, alkenyl, alkynyl, heteroalkyl, —OR^{A}, —C(O)R^{A}, —C(O)OR^{A}, —C(O)N(R^{C})(R^{D}), —N(R^{C})C(O)R^{A}, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵;

each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;

or R^{C} and R^{D}, taken together with the nitrogen atom to which they are attached, form a ring (e.g., a 5-7 membered ring), optionally substituted with one or more R⁶;

each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{E1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;

each R^{A1}, R^{B1}, R^{C1}, R^{D1}, R^{E1}, and R^{E1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;

each R⁷ is independently alkyl, alkenyl, alkynyl, heteroalkyl, halogen, cyano, oxo, hydroxyl, cycloalkyl, or heterocyclyl;

x is 1 or 2; and

y is 2, 3, or 4.

47. The implantable element of embodiment 46, wherein the compound of Formula (I) is a compound of Formula (I-b):

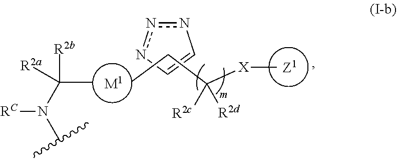

or a pharmaceutically acceptable salt thereof, wherein:

Ring M¹ is cycloalkyl, heterocyclyl, aryl, or heteroaryl, each of which is optionally substituted with 1-5 R³;

Ring Z¹ is cycloalkyl, heterocyclyl, aryl or heteroaryl, optionally substituted with 1-5 R⁵;
each of R^{2a}, R^{2b}, R^{2′}, and R^{2d} is independently hydrogen, alkyl, alkenyl, alkynyl, heteroalkyl, halo, cyano, nitro, amino, cycloalkyl, heterocyclyl, aryl, or heteroaryl, or
each of R^{2a} and R^{2b} or R^{2′} and R^{2d} is taken together to form an oxo group;

X is absent, N(R¹⁰)(R¹¹), O, or S;

R^{C} is hydrogen, alkyl, alkenyl, alkynyl, heteroalkyl, cycloalkyl, heterocyclyl, aryl, or heteroaryl, wherein each of alkyl, alkenyl, alkynyl, heteroalkyl, cycloalkyl, heterocyclyl, aryl, or heteroaryl is optionally substituted with 1-6 R⁶;

each R³, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each of R¹⁰ and R¹¹ is independently hydrogen, alkyl, alkenyl, alkynyl, heteroalkyl, —C(O)OR^{A1}, —C(O)R^{B1}, —OC(O)R^{B1}, —C(O)N(R^{C1}), cycloalkyl, heterocyclyl, aryl, or heteroaryl;

each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷;

each R⁷ is independently alkyl, alkenyl, alkynyl, heteroalkyl, halogen, cyano, oxo, hydroxyl, cycloalkyl, or heterocyclyl;

each m and n is independently 1, 2, 3, 4, 5, or 6; and

“ [symbol] ” refers to a connection to an attachment group or a polymer described herein.

48. The implantable element of embodiment 47, wherein the compound of Formula (I-b) is a compound of Formula (I-b-i):

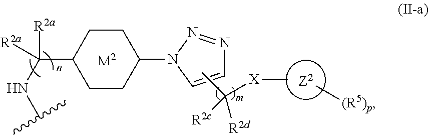

or a pharmaceutically acceptable salt thereof, wherein

Ring M² is aryl or heteroaryl optionally substituted with one or more R³;

Ring Z² is cycloalkyl, heterocyclyl, aryl, or heteroaryl;

each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group;

X is absent, O, or S;

each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; or two R⁵ are taken together to form a 5-6 membered ring fused to Ring Z²;

each R^{A1} and R^{B1} is independently hydrogen, alkyl, or heteroalkyl;

m and n are each independently 1, 2, 3, 4, 5, or 6;

p is 0, 1, 2, 3, 4, 5, or 6; and

“ [symbol] ” refers to a connection to an attachment group or a polymer described herein

49. The implantable element of embodiment 48, wherein the compound of Formula (I-b-i) is a compound of Formula (I-b-ii):

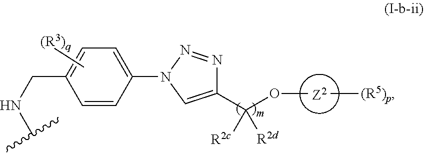

or a pharmaceutically acceptable salt thereof, wherein

Ring Z² is cycloalkyl, heterocyclyl, aryl or heteroaryl;

each of R^{2c} and R^{2d} is independently hydrogen, alkyl, or heteroalkyl, or R^{2c} and R^{2d} and taken together to form an oxo group;

each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen;

each R^{A1} and R^{B1} is independently hydrogen, alkyl, or heteroalkyl;

each of p and q is independently 0, 1, 2, 3, 4, 5, or 6; and “ [symbol] ” refers to a connection to an attachment group or a polymer described herein.

50. The implantable element of embodiment 46, wherein the compound of Formula (I) is a compound of Formula (I-c):

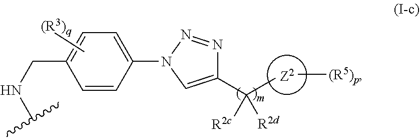

or a pharmaceutically acceptable salt thereof, wherein:

Ring Z² is cycloalkyl, heterocyclyl, aryl or heteroaryl;

each of R^{2c} and R^{2d} is independently hydrogen, alkyl, or heteroalkyl, or each of R^{2c} and R^{2d} is taken together to form an oxo group;

each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen;

each R^{A1} and R^{B1} is independently hydrogen, alkyl, or heteroalkyl;

m is 1, 2, 3, 4, 5, or 6;

each of p and q is independently 0, 1, 2, 3, 4, 5, or 6; and

“ [symbol] ” refers to a connection to an attachment group or a polymer described herein.

51. The implantable element of embodiment 46, wherein the compound of Formula (I) is a compound of Formula (I-d):

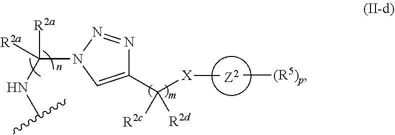

or a pharmaceutically acceptable salt thereof, wherein:

Ring Z² is cycloalkyl, heterocyclyl, aryl or heteroaryl;

X is absent, O, or S;

each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group;

each R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen;

each R^{A1} and R^{B1} is independently hydrogen, alkyl, or heteroalkyl;

each of m and n is independently 1, 2, 3, 4, 5, or 6;

p is 0, 1, 2, 3, 4, 5, or 6; and

“ [symbol] ” refers to a connection to an attachment group or a polymer described herein.

52. The implantable element of embodiment 46, wherein the compound of Formula (I) is a compound of Formula (I-e):

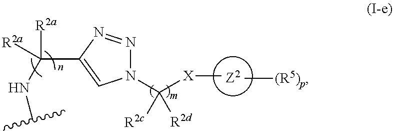

or a pharmaceutically acceptable salt thereof, wherein

Ring Z² is cycloalkyl, heterocyclyl, aryl or heteroaryl;

X is absent, O, or S;

each of R^{2a}, R^{2b}, R and R^{2d} is independently hydrogen, alkyl, or heteroalkyl, or each of R^{2a} and R^{2b} or R^{2c} and R^{2d} is taken together to form an oxo group;

each R⁵ is independently alkyl, heteroalkyl, halogen, oxo, —OR^{A1}, —C(O)OR^{A1}, or —C(O)R^{B1};

each R^{A1} and R^{B1} is independently hydrogen, alkyl, or heteroalkyl;

each of m and n is independently 1, 2, 3, 4, 5, or 6;

p is 0, 1, 2, 3, 4, 5, or 6; and

“ [symbol] ” refers to a connection to an attachment group or a polymer described herein.

53. The implantable element of embodiment 46, wherein the compound of Formula (I) is a compound of Formula (I-f):

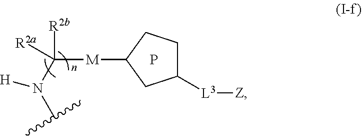

or a pharmaceutically acceptable salt thereof, wherein

M is alkyl optionally substituted with one or more R³;

Ring P is heteroaryl optionally substituted with one or more R⁴;

L³ is alkyl or heteroalkyl optionally substituted with one or more R²;

Z is alkyl, heteroalkyl, cycloalkyl, heterocyclyl, aryl, or heteroaryl, each of which is optionally substituted with one or more R⁵;

each of R^{2a} and R^{2b} is independently hydrogen, alkyl, or heteroalkyl, or R^{2a} and R^{2b} is taken together to form an oxo group;

each R², R³, R⁴, and R⁵ is independently alkyl, heteroalkyl, halogen, oxo, —OR^{A1}, —C(O)OR^{A1}, or —C(O)R^{B1};

each R^{A1} and R^{B1} is independently hydrogen, alkyl, or heteroalkyl;

n is independently 1, 2, 3, 4, 5, or 6; and

“ [symbol] ” refers to a connection to an attachment group or a polymer described herein.

54. The implantable element of embodiment 46, wherein the compound of Formula (I) is a compound of Formula (III-a):

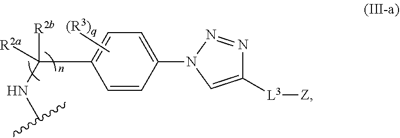

or a pharmaceutically acceptable salt thereof, wherein

L³ is alkyl or heteroalkyl, each of which is optionally substituted with one or more R²;

Z is hydrogen, alkyl, heteroalkyl, or —OR^{A}, wherein alkyl and heteroalkyl are optionally substituted with one or more R⁵;

each of R^{2a} and R^{2b} is independently hydrogen, alkyl, or heteroalkyl, or R^{2a} and R^{2b} is taken together to form an oxo group;

each R², R³, and R⁵ is independently alkyl, heteroalkyl, halogen, oxo, —OR^{A1}, —C(O)OR^{A1}, or —C(O)R^{B1};

R^{A} is hydrogen;

each R^{A1} and R^{B1} is independently hydrogen, alkyl, or heteroalkyl;

n is independently 1, 2, 3, 4, 5, or 6; and

“ [symbol] ” refers to a connection to an attachment group or a polymer described herein.

55. The implantable element of embodiment 46, wherein the compound of Formula (I) is a compound of Formula (III-b):

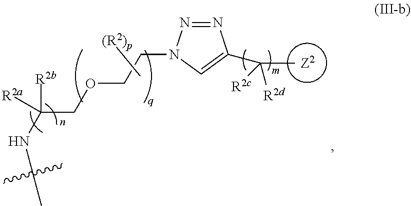

or a pharmaceutically acceptable salt thereof, wherein

Ring Z² is cycloalkyl, heterocyclyl, aryl, or heteroaryl, each of which is optionally substituted with 1-5 R⁵;

each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group;

each of R³ and R⁵ is independently alkyl, heteroalkyl, halogen, oxo, —OR^{A1}, —C(O)OR^{A1}, or —C(O)R^{B1};

each R^{A1} and R^{B1} is independently hydrogen, alkyl, or heteroalkyl;

m and n are each independently 1, 2, 3, 4, 5, or 6;

o and p are each independently 0, 1, 2, 3, 4, or 5;

q is an integer from 0 to 25; and

“ [symbol] ” refers to a connection to an attachment group or a polymer described herein.

56. The implantable element of any one of embodiments 46-55, wherein the compound of Formula (I) is a compound shown in Compound Table 1.
57. The implantable element of any one of embodiments 46-56, wherein the compound is selected from:

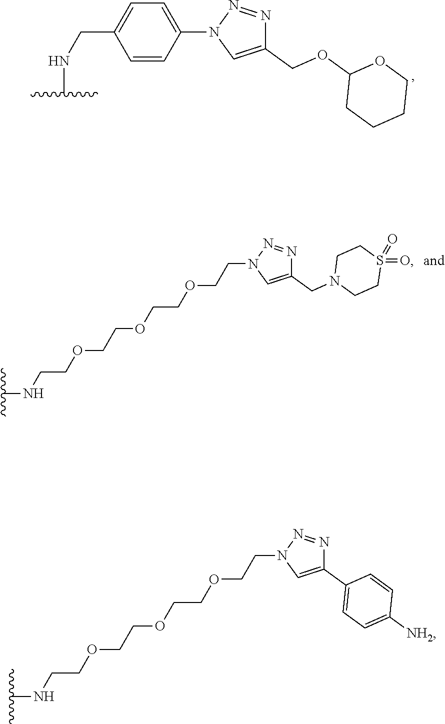 [SMILES: *NCc1ccc(-n2cc(COC3CCCCO3)nn2)cc1 ; *NCCOCCOCCOCCn1cc(CN2CCS(=O)(=O)CC2)nn1 ; *NCCOCCOCCOCCn1cc(-c2ccc(N)cc2)nn1]

or a salt thereof.
58. The implantable element of any one of embodiments 46-56, wherein the compound is selected from Compound 110, Compound 112, Compound 113, or Compound 114 from Compound Table 1.
59. The implantable element of any one of the preceding embodiments, wherein the implantable element is not substantially degraded after implantation in a subject for at least 30 days, 2 months, 3 months, 6 months, 9 months, or 12 months.
60. The implantable element of any one of the preceding embodiments, wherein the implantable element is removable from the subject without significant injury to the surrounding tissue, e.g., after about 5 days following implantation.
61. A method of treating a subject or supplying a product (e.g., a therapeutic product) to a subject, comprising:

administering or providing to the subject an implantable element or engineered MSFC of any one of embodiments 1 to 60, thereby treating the subject or supplying a product (e.g., a therapeutic product) to the subject.

62. The method of embodiment 61, comprising treating the subject.
63. The method of embodiment 62, comprising supplying a product (e.g., a therapeutic product) to the subject.
64. The method of any one of embodiments 61-63, wherein the subject is a human.
65. The method of any one of embodiments 61-64 wherein the engineered MSFCs are human cells (e.g., human MSFCs).
66. The method of any one of embodiments 61-65, wherein the polypeptide is an antibody (e.g., anti-nerve growth factor antibody), an enzyme (e.g., alpha-galactosidase or a clotting factor (e.g., a blood clotting factor, e.g., an activated blood clotting factor).
67. The method of any one of embodiments 61-66, wherein the plurality of engineered MSFCs or the implantable element is provided as a treatment for a disease.
68. The method of embodiment 67, wherein the disease is a blood clotting disease or a lysosomal storage disease (e.g., a hemophilia (e.g., Hemophilia A or Hemophilia B), Fabry Disease, Gaucher Disease, Pompe Disease, or MPS I).
69. The method of embodiment 67, wherein the disease is diabetes.
70. The method of embodiment 67, wherein the disease is not diabetes.
71. The method of any one of embodiments 61-68, wherein the implantable element is administered to a first subject having less than about 50%, 40%, 30%, 25%, 20%, 15%, 10%, 5%, 2%, or 1% of the polypeptide (e.g., a blood clotting factor, e.g., Factor I, Factor II, Factor V, Factor VII, Factor VIII, Factor IX, Factor X, Factor XI, or Factor XIII) relative to a second subject (e.g., a healthy subject), e.g., as determined by a blood test.
72. The method of any one of embodiments 61-71, wherein the level of a biomarker (e.g., a serum biomarker) in a subject is monitored, e.g., in order to determine the level of efficacy of treatment.
73. The method of any one of embodiments 71-73, wherein the implantable element is administered to, implanted in, or provided to a site other than the central nervous system, brain, spinal column, eye, or retina.
74. The method of any one of embodiments 61-72, wherein the implantable element is administered to, implanted in, or provided to a site at least about 1, 2, 5, or 10 centimeters from the central nervous system, brain, spinal column, eye, or retina.
75. A method of making or manufacturing an implantable element comprising a plurality of engineered MSFCs, comprising:

providing a plurality of engineered MSFCs, e.g., engineered MSFCs described herein, and

disposing the plurality of engineered MSFCs in an enclosing component, e.g., an enclosing component described herein,

thereby making or manufacturing the implantable element.
76. A method of evaluating an implantable element comprising a plurality of engineered MSFCs, comprising:

providing an implantable element comprising a plurality of engineered MSFCs described herein; and

evaluating a structural or functional parameter of the implantable element or the plurality of engineered MSFCs,

thereby evaluating an implantable element.
77. The method of embodiment 76, comprising culturing the plurality of engineered MSFCs in vitro or culturing the engineered MSFCs or plurality of engineered MSFCs in an animal, e.g., a non-human animal, or a human subject.
78. The method of embodiment 76-77, comprising evaluating the plurality of engineered MSFCs, for one or more of:

viability;

the production of an engineered polypeptide;

the production of an engineered RNA;

the uptake of a nutrient or of oxygen; or

the production of a waste product.

79. The method of any one of embodiments 75-78, further comprising: formulating the implantable element into a drug product if one or more of: the viability; production of an engineered polypeptide; the production of an engineered RNA; the uptake of a nutrient or of oxygen; or the production of a waste product meets a predetermined value.
80. The method of any one of embodiments 75-79, comprising evaluating a parameter of the cells related to a form factor, e.g., a form factor described herein.
81. The method of any of embodiments 76-80, wherein the evaluation is performed at least 1, 5, 10, 20, 30, or 60 days after disposing the plurality of engineered MSFCs in the implantable element.
82. The method of any one of embodiments 76-80, wherein the evaluation is performed at least 1, 5, 10, 20, 30, or 60 days after the initiation of culturing the engineered MSFCs.
83. A method of monitoring an implantable element of any one of embodiments 1 to 68, comprising:

obtaining, e.g., by testing the subject or a sample therefrom, the level of a component (e.g., a polypeptide) released by the plurality of engineered MSFCs in the subject, or

obtaining, e.g., by testing the subject or a sample therefrom, the level of a product dependent on the activity of the component,

thereby monitoring or evaluating an implantable element.
84. The method of embodiment 83, wherein the component is measured in the peripheral circulation, e.g., in the peripheral blood.
85. The method of any one of embodiments 89-93, wherein the level of the component (e.g., polypeptide) is compared with a reference value.
86. The method of any one of embodiments 83-85, wherein responsive to the level or the comparison, the subject is classified, e.g., as in need of or not in need of an additional implantable element or additional engineered MSFCs.
87. The method of any one of embodiments 83-86, the method comprises (e.g., responsive to the level or comparison), retrieving the implantable element or engineered MSFCs from the subject.
88. The method of any one of embodiments 83-87, the level is obtained from about 1 hour to about 30 days to after administering (e.g., implanting or injecting) an implantable element or engineered MSFCs or about 1 hour to about 30 days after a prior evaluation.
89. A plurality of MSFCs having a preselected form factor or a form factor disclosed herein.
90. The plurality of MSFCs of embodiment 89, wherein the form factor comprises a cluster of engineered MSFCs.
91. The plurality of MSFCs of embodiment 90, wherein the cluster comprises at least about 100, 200, 300, 400, or 500 MSFCs.
92. A substrate comprising a plurality of chambers, each chamber of the plurality containing an MSFC or an engineered MSFC.
93. The substrate of embodiment 92, wherein each chamber of the plurality of chambers comprises a plurality of MSFCs or engineered MSFCs, e.g., a plurality of engineered MSFCs having a form factor described herein, e.g., a cluster.
94. A microcarrier (e.g., a bead or a matrix), having disposed thereon an engineered MSFC described herein or a cluster of MSFCs (e.g., engineered MSFCs).
95. The microcarrier of embodiment 94, wherein the microcarrier comprises a polystyrene bead.
96. A preparation of engineered MSFCs (e.g., engineered MSFCs), wherein the preparation comprises at least about 10,000; 15,000; 20,000; 25,000; 30,000; 40,000; 50,000; 60,000; or 75,000 engineered MSFCs (e.g., MSFCs as described herein).
97. A pharmaceutical composition comprising a plurality of the implantable element or engineered MSFC of any one of embodiments 1 to 68.

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present disclosure, as defined in the following claims.

1. An implantable element comprising a plurality of engineered mesenchymal stem function cells (MSFCs), e.g., engineered MSFCs, comprising an exogenous nucleic acid which promotes and/or conditions the production of a polypeptide, e.g., a therapeutic polypeptide, wherein:
a) the plurality (e.g., plurality of engineered MSFCs) produces or releases the polypeptide for at least 5 days, e.g., when implanted into a subject or when evaluated by a reference method, e.g., microscopy or Western blotting;
b) the plurality (e.g., plurality of engineered MSFCs) produces or releases at least 10 picograms of the polypeptide per day, e.g., produces at least 10 picograms of the polypeptide per day for at least 5 days, e.g., when implanted into a subject or when evaluated by a reference method, e.g., microscopy or Western blotting;
c) the plurality (e.g., plurality of engineered MSFCs) produces or releases the polypeptide at a rate, e.g., of at least 10 picograms of polypeptide per day, which is at least 50% (e.g., at least 60%, at least 70%, at least 80%, at least 90%, at least 95%, or at least 99%) of the rate control cells produce when, e.g., not encapsulated in the implantable element or not embedded or implanted in a subject, e.g., as evaluated by microscopy or Western blotting;
d) the plurality (e.g., plurality of engineered MSFCs) produces or releases the polypeptide for at least 5 days and the amount released per day does not vary more than 50% (e.g., at least about 40%, about 30%, about 20%, about 10%, about 5%, or less), e.g. as evaluated by microscopy or Western blotting;
e) upon introduction into a subject, sufficient polypeptide is produced or released such that a location at least about 5 cm, about 10 cm, about 25 cm, about 50 cm, or about 100 cm away receives an effective concentration (e.g., a therapeutically effective concentration) of the polypeptide (e.g., a therapeutically effective concentration found in the pancreas, liver, blood, or outside the eye);
f) sufficient polypeptide is produced or released such that when embedded or implanted in the peritoneal cavity of a subject, e.g., a detectable level of the polypeptide, e.g., 10 picograms, is found at a location at least 5 cm, 10 cm, 25 cm, 50 cm, or 100 cm away from the engineered MSFC (e.g., engineered MSC);
g) upon introduction into a subject, sufficient polypeptide is produced or released such that about 50% of the polypeptide produced or released (about 60%, about 70%, about 80%, about 90%, or about 99% of the therapeutic polypeptide produced or released) enters the circulation (e.g., peripheral circulation) of a subject;
h) the plurality (e.g., plurality of engineered MSFCs) is capable of phagocytosis, e.g., is capable of about 99%, about 95%, about 90%, about 85%, about 80%, about 75%, about 70%, about 60%, or about 50% of the level of phagocytosis compared with reference non-engineered MSFCs, e.g., as evaluated by microscopy or Western blotting;
i) the plurality (e.g., plurality of engineered MSFCs) is capable of autophagy, e.g., is capable of about 99%, about 95%, about 90%, about 85%, about 80%, about 75%, about 70%, about 60%, or about 50% of the level of autophagy compared with reference non-engineered MSFCs, e.g., as evaluated by microscopy or Western blotting;
j) the plurality (e.g., plurality of engineered MSFCs) is provided having a form factor described herein, e.g., as a cluster, spheroid, or aggregate of engineered MSFCs;
k) the plurality (e.g., plurality of engineered MSFCs) is disposed on a non-cellular carrier (e.g, a microcarrier, e.g., a bead, e.g., a polyester, polystyrene, or polymeric bead);
l) the plurality (e.g., plurality of engineered MSFCs) is capable of proliferating after encapsulation in the implantable element, e.g., as determined by microscopy;
m) the plurality (e.g., plurality of engineered MSFCs) is capable of not proliferating after encapsulation in the implantable element, e.g., as determined by microscopy; or
n) upon introduction, administration, or implantation into a subject, sufficient polypeptide is produced or released such that an effective concentration (e.g., a therapeutically effective concentration) of the polypeptide is found in the peripheral bloodstream (e.g., a therapeutically effective concentration found in the pancreas, liver, blood, or outside the eye),
and the implantable element is modified with a compound of Formula (I): 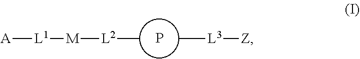
or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₂-C₆-alkenylene)-, —N(R^{C})N(R^{D})—, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, wherein each alkyl, alkenyl, alkynyl, alkylene, alkenylene, heteroalkyl, cycloalkyl, heterocyclyl, aryl, and heteroaryl is linked to an attachment group (e.g., an attachment group defined herein) and is optionally substituted by one or more R¹;
each of L¹ and L³ is independently a bond, alkyl, or heteroalkyl, wherein each alkyl and heteroalkyl is optionally substituted by one or more R²;
L² is a bond;
M is absent, alkyl, heteroalkyl, cycloalkyl, heterocyclyl, aryl, or heteroaryl, each of which is optionally substituted by one or more R³;
P is absent, cycloalkyl, heterocycyl, or heteroaryl each of which is optionally substituted by one or more R⁴;
Z is hydrogen, alkyl, alkenyl, alkynyl, heteroalkyl, —OR^{A}, —C(O)R^{A}, —C(O)OR^{A}, —C(O)N(R^{C})(R^{D}), —N(R^{C})C(O)R^{A}, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring (e.g., a 5-7 membered ring), optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{E1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

2. The implantable element of claim 1, wherein the engineered MSFCs (e.g., engineered MSCs) are human cells (e.g., a human MSCs).

3. The implantable element of any one of the preceding claims, wherein the exogenous nucleic acid encodes the polypeptide.

4. The implantable element of any one of the preceding claims, wherein the polypeptide is an enzyme (e.g., alpha-galactosidase) or a clotting factor (e.g., a blood clotting factor, e.g., an activated blood clotting factor).

5. The implantable element of claim 4, wherein the polypeptide comprises Factor I, Factor II, Factor V, Factor VII, Factor VIII, Factor IX, Factor X, Factor XI, or Factor XIII.

6. The implantable element of any one of claims 3-5, wherein the sequence of the polypeptide comprises at least one amino acid deletion, addition, or substitution relative to the sequence (e.g., naturally occurring human sequence) of Factor I, Factor II, Factor V, Factor VII, Factor VIII, Factor IX, Factor X, Factor XI, or Factor XIII.

7. The implantable element of any one of claims 3-6, wherein the polypeptide comprises a naturally occurring human Factor VIII amino acid sequence (or a variant thereof) or a naturally occurring human Factor IX amino acid sequence (or a variant thereof).

8. The implantable element of any one of claims 3-7, wherein the polypeptide comprises SEQ ID NO:1 or a variant thereof.

9. The implantable element of any one of claims 3-8, wherein the polypeptide comprises SEQ ID NO: 2 or a variant thereof, e.g., a alanine substituted for threonine at amino acid position 148 of SEQ ID NO:2 or a leucine substituted for arginine at amino acid position 338 of SEQ ID NO:2.

10. The implantable element of any one of claims 3-9, wherein the polypeptide comprises insulin (e.g, insulin A-chain, insulin B-chain, or proinsulin).

11. The implantable element of any one of claims 3-10, wherein the polypeptide is a replacement therapy or a replacement protein (e.g., a clotting factor, an enzyme, or an antibody).

12. The implantable element of any one of claims 3-11, wherein the polypeptide (e.g., a blood clotting factor) is glycosylated.

13. The implantable element of any one of the preceding claims, wherein the engineered MSFCs are provided as a treatment for a disease.

14. The implantable element of claim 13, wherein the disease is a blood clotting disease or a lysosomal storage disease (e.g., a hemophilia (e.g., Hemophilia A or Hemophilia B), Fabry Disease, Gaucher Disease, Pompe Disease, or MPS I).

15. The implantable element of any one of claims 13-14, wherein the disease is diabetes.

16. The implantable element of any one of the preceding claims, wherein the engineered MSFCs are provided as a prophylactic treatment.

17. The implantable element of any one of the preceding claims, wherein the implantable element is formulated for injection into a subject (e.g., intraperitoneal, intramuscular, or subcutaneous injection).

18. The implantable element of any one of the preceding claims, wherein the implantable element is formulated for implantation into a subject (e.g., into the peritoneal cavity, e.g., the lesser sac).

19. The implantable element of any one of the preceding claims, wherein the implantable element is implanted or injected into the lesser sac, into the omentum, or into the subcutaneous fat of a subject.

20. The implantable element of any one of the preceding claims, wherein the implantable element is administered to a first subject having less than about 50%, 40%, 30%, 25%, 20%, 15%, 10%, 5%, 2%, or 1% of the polypeptide (e.g., a blood clotting factor, e.g., Factor I, Factor II, Factor V, Factor VII, Factor VIII, Factor IX, Factor X, Factor XI, or Factor XIII) relative to a second subject (e.g., a healthy subject), e.g., as determined by a blood test.

21. The implantable element of any one of the preceding claims, wherein the level of a biomarker (e.g., a serum biomarker) in a subject is monitored, e.g., in order to determine the level of efficacy of treatment.

22. The implantable element of any one of the preceding claims, wherein the implantable element comprises a cluster of engineered MSFC, a plurality of engineered MSFCs, or a microcarrier (e.g., a bead or matrix) comprising an MSFC or MSFCs or a plurality of engineered MSFCs.

23. The implantable element of claim 22, wherein the plurality of engineered MSFCs, a plurality of engineered MSFCs, or a microcarrier (e.g., a bead or matrix) comprising an MSFC or MSFCs produces a plurality of polypeptides.

24. The implantable element of any one of the preceding claims, wherein the implantable element comprises an enclosing component.

25. The implantable element of claim 24, wherein the enclosing component is formed in situ on or surrounding an engineered MSFC, a plurality of engineered MSFCs, or a microcarrier (e.g., a bead or matrix) comprising an MSFC or MSFCs.

26. The implantable element of claim 24, wherein the enclosing component is preformed prior to combination with the enclosed engineered MSFC, a plurality of engineered MSFCs, or a microcarrier (e.g., a bead or matrix) comprising an MSFC or MSFCs.

27. The implantable element of any one of claims 24-26, wherein the enclosing component comprises a flexible polymer (e.g., PLA, PLG, PEG, CMC, or a polysaccharide, e.g., alginate).

28. The implantable element of any one of claims 24-27, wherein the enclosing component comprises an inflexible polymer or metal housing.

29. The implantable element of any one of the preceding claims, wherein the implantable element is chemically modified.

30. The implantable element of any one of claims 25-29, wherein the enclosing component is chemically modified.

31. The implantable element of any one of the preceding claims, wherein the compound of Formula (I) is a compound of any one of Formulas (I-a), (I-b), (I-c), (I-d), (I-e), (I-f), (II), (II-a), (III), (III-a), (III-b), (III-c), or (III-d), or a pharmaceutically acceptable salt thereof.

32. The implantable element of any one of the preceding claims, wherein the compound of Formula (I) is a compound shown in Table 1.

33. The implantable element of any one of the preceding claims, wherein the compound is selected from: 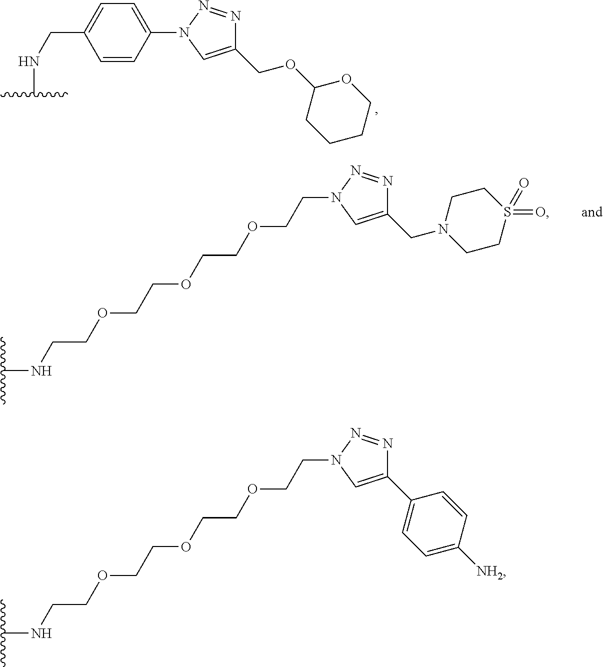
or a salt thereof.

34. The implantable element of any one of the preceding claims, wherein the compound is selected from Compound 110, Compound 112, Compound 113, or Compound 114 from Table 1.

35. The implantable element of any one of the preceding claims, wherein the subject is a human.

36. A composition for use in treating a subject having a disease or disorder comprising:
administering or providing to the subject an implantable element described herein, e.g., any one of claims 1-35, thereby treating the subject.

37. The method of claim 36, wherein the subject is a human.

38. The method of any one of claims 36-37, wherein the engineered MSFCs are human cells (e.g., human MSFCs).

39. The method of any one of claims 36-38, wherein the product is an exogenous nucleic acid which promotes and/or conditions the production of a polypeptide, e.g., a therapeutic polypeptide.

40. The method of claim 39, wherein:
a) the plurality of engineered MSFCs (e.g., engineered MSCs) or implantable element produces or releases the polypeptide for at least 5 days, e.g., when implanted into a subject or when evaluated by a reference method, e.g., microscopy or Western blotting;
b) the plurality of engineered MSFCs (e.g., engineered MSCs) or implantable element produces or releases at least 10 picograms of the polypeptide per day, e.g., produces at least 10 picograms of the polypeptide per day for at least 5 days, e.g., when implanted into a subject or when evaluated by a reference method, e.g., microscopy or Western blotting;
c) the plurality of engineered MSFCs (e.g., engineered MSCs) produces or releases the polypeptide at a rate, e.g., of at least 10 picograms of polypeptide per day, which is at least 50% (e.g., at least 60%, at least 70%, at least 80%, at least 90%, at least 95%, or at least 99%) of the rate of control cells not encapsulated in the implantable element or not embedded or implanted in a subject, e.g., as evaluated by microscopy or Western blotting;
d) the plurality of engineered MSFCs (e.g., engineered MSCs) produces or releases the polypeptide for at least 5 days and the amount released per day does not vary more than 50% (e.g., at least about 40%, about 30%, about 20%, about 10%, about 5%, or less), e.g. as evaluated by microscopy or Western blotting;
e) upon introduction into a subject, sufficient polypeptide is produced or released such that a location at least about 5 cm, about 10 cm, about 25 cm, about 50 cm, or about 100 cm away receives an effective concentration (e.g., a therapeutically effective concentration) of the polypeptide (e.g., a therapeutically effective concentration found in the pancreas, liver, blood, or outside the eye);
f) sufficient polypeptide is produced or released such that when embedded or implanted in the peritoneal cavity of a subject, e.g., a detectable level of the polypeptide, e.g., 10 picograms, is found at a location at least 5 cm, 10 cm, 25 cm, 50 cm, or 100 cm away from the engineered MSFC (e.g., engineered MSC);
g) upon introduction into a subject, sufficient polypeptide is produced or released such that about 50% of the polypeptide produced or released (about 60%, about 70%, about 80%, about 90%, or about 99% of the therapeutic polypeptide produced or released) enters the circulation (e.g., peripheral circulation) of a subject;
h) the plurality of engineered MSFCs (e.g., engineered MSCs) is capable of phagocytosis, e.g., is capable of about 99%, about 95%, about 90%, about 85%, about 80%, about 75%, about 70%, about 60%, or about 50% of the level of phagocytosis compared with non-engineered MSFC (e.g., non-engineered MSC), e.g., as evaluated by microscopy or Western blotting;
i) the plurality of engineered MSFCs (e.g., engineered MSCs) is capable of autophagy, e.g., is capable of about 99%, about 95%, about 90%, about 85%, about 80%, about 75%, about 70%, about 60%, or about 50% of the level of autophagy compared with non-engineered MSFC (e.g., engineered MSC), e.g., as evaluated by microscopy or Western blotting;
j) the plurality of engineered MSFCs (e.g., engineered MSCs) is provided having a form factor described herein, e.g., as a cluster, spheroid, or aggregate of MSFC (e.g., engineered MSC);
k) the plurality of engineered MSFCs (e.g., engineered MSCs) is disposed on a non-cellular carrier (e.g, a microcarrier, e.g., a bead, e.g., a polyester, polystyrene, or polymeric bead);
l) the plurality of engineered MSFCs (e.g., engineered MSCs) proliferates or is capable of proliferating after encapsulation in the implantable element, e.g., as determined by microscopy;
m) the plurality of engineered MSFCs (e.g., engineered MSCs) does not proliferate or is not capable of proliferating after encapsulation in the implantable element, e.g., as determined by microscopy; or
n) upon introduction, administration, or implantation into a subject, sufficient polypeptide is produced or released such that an effective concentration (e.g., a therapeutically effective concentration) of the polypeptide is found in the peripheral bloodstream of the subject (e.g., a therapeutically effective concentration found in the pancreas, liver, blood, or outside the eye).

41. The method of any one of claims 39-40, wherein the polypeptide comprises a clotting factor (e.g., a blood clotting factor, e.g., an activated blood clotting factor).

42. The method of any one of claims 39-41, wherein the polypeptide comprises a naturally occurring amino acid sequence for Factor I, Factor II, Factor V, Factor VII, Factor VIII, Factor IX, Factor X, Factor XI, or Factor XIII, or a variant of any of said sequences.

43. The method of any one of claims 39-42, wherein the polypeptide comprises a naturally occurring human Factor VIII amino acid sequence or a variant thereof or comprises a naturally occurring human Factor IX amino acid sequence or a variant thereof.

44. The method of any one of claims 39-43, wherein the polypeptide comprises SEQ ID NO:1 or a variant thereof.

45. The method of any one of claims 39-44, wherein the polypeptide comprises SEQ ID NO: 2 or a variant thereof, e.g., an alanine substituted for threonine at amino acid position 148 of SEQ ID NO:2 or a leucine substituted for arginine at amino acid position 338 of SEQ ID NO:2.

46. The method of any one of claims 39-45, wherein the sequence of the polypeptide comprises at least one amino acid deletion, addition, or substitution relative to the sequence (e.g., naturally occurring human sequence) of Factor I, Factor II, Factor V, Factor VII, Factor VIII, Factor IX, Factor X, Factor XI, or Factor XIII.

47. The method of any one of claims 39-40, wherein the polypeptide comprises insulin (e.g., insulin A-chain, insulin B-chain, or proinsulin).

48. The method of any one of claims 39-47, wherein the polypeptide is a replacement therapy or a replacement protein (e.g., a clotting factor, an enzyme, or an antibody).

49. The method of any one of claims 39-48, wherein the plurality of engineered MSFCs or the implantable element is provided as a treatment for a disease.

50. The method of claim 49, wherein the disease is a blood clotting disease or a lysosomal storage disease (e.g., a hemophilia (e.g., Hemophilia A or Hemophilia B), Fabry Disease, Gaucher Disease, Pompe Disease, or MPS I).

51. The method of any one of claims 49-50, wherein the implantable element is formulated for injection into a subject (e.g., intra-omentum, intra subcutaneous fat, intraperitoneal, intramuscular, or subcutaneous injection).

52. The method of any one of claims 49-51, wherein the implantable element is formulated for implantation into a subject (e.g., into the peritoneal cavity, e.g., the lesser sac, into the omentum, or into the subcutaneous fat).

53. The method of any one of claims 49-52, wherein the level of a biomarker (e.g., a serum biomarker) in a subject is monitored, e.g., in order to determine the level of efficacy of treatment.

54. A method of making or manufacturing an implantable element comprising a plurality of engineered MSFCs (e.g., engineered MSCs), comprising:
providing a plurality of engineered MSFCs (e.g., engineered MSCs), e.g., engineered MSFCs described herein, and
disposing the plurality of engineered MSFCs (e.g., engineered MSCs) in an enclosing component, e.g., an enclosing component described herein,
thereby making or manufacturing the implantable element.
